(19) United States
(12) Patent Application Publication
(10) Pub. No.: US 2026/0117001 A1
(43) Pub. Date: Apr. 30, 2026

(54) FLUOROPHORE-POLYMER CONJUGATES AND USES THEREOF

(71) Applicant: ERISYON INC., Austin, TX (US)

(72) Inventors: Christopher MARTIN, Austin, TX (US); Tucker FOLSOM, Austin, TX (US); Jagannath SWAMINATHAN, Austin, TX (US)

(21) Appl. No.: 19/117,778

(22) PCT Filed: Oct. 2, 2023
(86) PCT No.: PCT/US2023/075739

§ 371 Date: Apr. 2, 2025

Related U.S. Application Data
(60) Provisional application No. 63/412,780, filed on Oct. 3, 2022.
(60) Provisional application No. 63/582,766, filed on Sep. 14, 2023.

(51) Int. Cl. C07K 17/08; G01N 33/543; G01N 33/58; G01N 33/68

(52) U.S. Cl. CPC C07K 17/08; G01N 33/54306; G01N 33/54353; G01N 33/582; G01N 33/6824

(57) ABSTRACT

The present disclosure relates to polymers that include a functional group configured to couple to a polypeptide or protein; a backbone; and an amino acid residue conjugated to a fluorophore. In some embodiments, the polymers may be used in fluorosequencing methods.

### REFERENCE TO AN ELECTRONIC SEQUENCE LISTING

The contents of the electronic sequence listing (401WO_SeqListing.xml; Size: 59,867 bytes; and Date of Creation: Sep. 29, 2023) are herein incorporated by reference in their entirety.

### BACKGROUND

Many diseases arise due to perturbations in the levels of multiple proteins, their modifications, and their interactions, so itis desirable to accurately measure them in order to diagnose and/or treat such diseases. Fluorosequencing is a highly parallelized single molecule peptide sequencing platform, based on determining the sequence positions of select amino acid types within peptides to enable their identification and quantification from a reference database.

### BRIEF SUMMARY

In some aspects, the present disclosure provides a polymer including a functional group configured to couple to a polypeptide or protein; a backbone; and an amino acid residue conjugated to a fluorophore.

Another embodiment of the present disclosure is a polymer including a functional group configured to couple to a polypeptide or protein; a backbone; and an amino acid residue conjugated to a fluorophore, wherein the backbone includes a rigid polypeptide comprising at least ten amino acid residues. Optionally in this embodiment, or any other embodiment disclosed herein, the backbone further comprises a flexible spacer. Optionally in this embodiment, or any other embodiment of the present disclosure, the polymer includes the functional group, the flexible spacer, the rigid polypeptide and the amino acid residue conjugated to a fluorophore in amino-terminal to carboxy-terminal direction or carboxy-terminal to amino-terminal direction.

Another embodiment of the present disclosure is a method of reducing dye-dye interactions, including a) providing a polymer including a functional group configured to couple to a polypeptide or protein, a backbone, and an amino acid residue conjugated to a fluorophore; b) providing a biomolecule including at least two reactive groups; and c) attaching at least two polymers to the at least two reactive groups via the functional group, wherein the two polymers include the same fluorophore; thereby reducing dye-dye interactions between identical fluorophores compared to dye-dye interactions between identical fluorophores conjugated directly to the at least two reactive groups of the biomolecule.

### BRIEF DESCRIPTION OF THE DRAWINGS

FIGS. 1A-1B depict an example polymer (or “tether”) of the present disclosure including a rigid polypeptide (for example, a polyproline Pro30 helix), a flexible spacer (for example, a flexible PEG linker), a functional group (for example, a reactive (clack) chemical group) and a fluorophore. FIG. 1A depicts a chemical structure of an example polymer (SEQ ID NO:35). FIG. 1B shows a simplified schematic representation of an example polymer.

FIG. 2 depicts a schematic representation of an example bottle brush polymer of the present disclosure.

FIG. 3 depicts an example polymer of the present disclosure of the formula DBCO-Gly-(O—CH₂—CH₂)₂-Gly-Pro₃₀-Lys(JFX554)-CONH₂ (SEQ ID NO:35).

FIG. 4 depicts an example of a flexible spacer of the present disclosure including a flexible PEG polymer with 23 monomer PEG subunits.

FIGS. 5A-5B depict an example of a structure of a polymer including a charged positive (arginine; FIG. 5A; SEQ ID NO:48) or negative (phenylsulfonic acid; FIG. 5B) species.

FIG. 6 depicts an overview of fluorosequencing technology highlighting the improvements and developments of each workflow in the process. Improvements were implemented to the sample preparation, imaging, fluidics, image processing, and peptide-read matching workflows. To improve sample preparation, 70 dyes were screened for improved photophysical properties and chemical stability to Edman solvents, selecting dyes across 6 fluorescent channels. Dye-dye interactions on fluorescently labeled peptides were mitigated through spacing fluorophores from the peptide backbone using long polyproline rigid polymers (or “promers”). Solvents and conditions for increasing Edman efficiency in less time were improved, and non-specific binding to flow cell surfaces was decreased by 23-fold by immobilizing peptides on azide-derivatized surfaces using click chemistry. Finally, a scalable image processing pipeline and a novel machine learning classifier framework was developed and implemented to infer peptide identities from raw reads.

FIGS. 7A-7D depict fluorosequencing using Atto643 and TexasRed dyes, which were selected as improved dyes for fluorosequencing through a controlled set of single molecule experiments. Atto643 and TexasRed dyes were downselected through estimating and comparing parameters from controlled set of experiments. FIG. 7A depicts a comparison of the dye-destruction rate through cycles of Edman chemistry on acetylated peptides (JSP260 and JSP288) carrying their respective fluorophores, showing the rates are 5.6% and 2% per cycle, respectively. FIG. 7B depicts a comparison of photobleaching rates between these peptides, which shows that less than 1.1% and 19% of Atto643 and Texas Red dyes, respectively, photobleach in 15 imaging cycles. FIGS. 7C-7D depict the mean intensity (mu) and the spread (sigma) of TexasRed (FIG. 7C) and Atto643 dye (FIG. 7D), which are 4970.85 and 0.19 AU for TexasRed and 11729.35 and 0.22 for Atto643 dye, respectively.

FIG. 8 depicts solvent stable fluorophores selected to span the visible spectra. To enable fluorosequencing, multiple fluorophores are needed that can be distinguishable across the visible spectra. Through screening of 70 dyes for solvent stability, four different fluorophores were identified, Atto425, Atto495, TexasRed, and Atto643, for the microscope imaging setup (see Example 8, Materials and Methods). The excitation and emission spectra for each of these fluorophores are shown.

FIGS. 9A-9B depict improvement of coupling solvent and time for cleavage chemistry, which increased Edman efficiency to >95% across a range of different peptides. FIG. 9A depicts the normalized counts of fluorescently labeled amino acid (SEQ ID NOs:17-22) cleaved at the correct cycle, this increased with increasing time of TFA incubation, with the maximum cleavage rate observed with an 8-minute trifluoroacetic acid incubation time. FIG. 9B depicts the addition of N-methylmorpholine into the PITC coupling solution, which increased the Edman efficiency by 12% (depicted as drop percentage of fluorescent tracks at 2nd position) for peptide JSP127 (SEQ ID NO:17).

FIGS. 10A-10C depict how improvements of Edman conditions and solvents increased the Edman efficiency across multiple amino acids and peptide sequences.

FIGS. 10A-10B depict lysine residues at the third amino acid sequence position that were fluorescently labeled in two peptides that contained either a preceding N-terminal proline (in JSP263; FIG. 10A (SEQ ID NO:19)) or glycine (in JSP254; FIG. 10B (SEQ ID NO:18)) residue. Following Edman improvement procedures, the largest fluorescence intensity drop per molecule occurred at the expected sequence positions in 63% and 74% of peptides, respectively. FIG. 10C shows an average of 63-75% of peptides was observed with differing N-terminal amino acid sequences correctly showing the largest fluorescence intensity drop at the labeled lysine residue. These rates correspond approximately to an Edman efficiency of 91-99% per cycle.

FIGS. 11A-11B illustrate how the position of prolines with respect to the labeled amino acid effects the efficiency of Edman degradation. The efficiency at which the Atto643 labeled lysine residue is cleaved was seen to be affected by the presence of proline residues by an average of 9.5%, when proline residues were located N-terminal to the fluorescently labeled lysine residue. Fluorosequencing for two sets of similar peptides was compared, differing in the position of proline residue at the 2nd (FIG. 11A; peptides—JSP286 (SEQ ID NO:25), JSP263 (SEQ ID NO:19)) or 3rd position (FIG. 11B; peptides—JSP285 (SEQ ID NO:23), JSP287 (SEQ ID NO:24)), and the decrease in efficiency was found to be similar.

FIG. 12A-12B depict a vapor deposition method for silanization of glass slides, which reduces fluorescent contamination across the different imaging channels. FIG. 12A shows the image setup for vapor deposition of 3-azidopropylsilane (see methods). FIG. 12B shows the fluorescent images of the slide post functionalization, which have extremely low counts of fluorescent contaminants across the four imaging channels (445, 480, 532 and 561 channel; see methods for optical setup). The values images represent the number of peaks/field for each channel. The peptides in the 640 channel (not shown) contain a dye-labeled peptide which is used to focus the slide. Scalebar represents 10 μm.

FIGS. 13A-13D depict quenching of like fluorophores labeled on the same peptide. By measuring the intensity distributions for single, two, three and four fluorophores and fitting them through an additive gaussian distribution, it was observed that there were significant dye-dye interactions or quenching of fluorescent signal between the fluorophores. The raw intensities for the four different peptides are shown in FIGS. 13A-13D, with an overlay of the predicted intensity distribution based on the gaussian fit parameters for the single dye.

FIGS. 14A-14D depict the observation of FRET across a wide range of donor fluorophores. FIGS. 14A-14B show FRET phenomena observed in a peptide containing JF549 and Atto647N (JSP129; SEQ ID NO:29). FIG. 14A: Overlay and offset images of the peptides across three channels—(1) 647, (2) “FRET” and (3) 561 channel to indicate the missing signal in the 561 or donor channel. FIG. 14B: Recovery of the counts of the 561 channel after photobleaching of the dyes in the 647 channel can be seen through the raw images of the donor and the acceptor channels before and after photobleaching FIGS. 14C-14D: FRET phenomena is also observed across multiple combinations of dye-pairs—Alexa488/Atto647N (FIG. 14C) and JF525/Atto647N (FIG. 14D). Despite the minimal overlap between the emission spectra of the Alexa488 (FIG. 14C) and JF525 (FIG. 14D) dyes with the Atto647N dyes with estimated FRET efficiency of 32.2% and 37% (as calculated using the online FRET calculator-https://www.fpbase.org/fret/), significantly high FRET signal is observed.

FIGS. 15A-15B show data to demonstrate that FRET is mitigated between fluorophores on peptides, when attached through polyproline linkers. FIG. 15A: Donor signal of tetramethylrhodamine recovers when spaced away from Atto647N dye using rigid polyproline linker. The donor fluorophore (tetramethylrhodamine) was excited on two peptides (depicted in the legend in the left panel) using a 500 nm monochromator. The emission signal was recorded from 525-700 nm and it was found that the tetramethylrhodamine dye spectrum was absent for the shorter Pro (3) peptide while present in the Pro (14) (Peptide-JSP168). FIG. 15B: Increased polyproline linker length to 30 units decreased FRET efficiency to <10% on the single molecule imaging system. Single molecule imaging was performed on three peptides, JSP212, JSP213, and JSP214, with different constructions of donor fluorophore (Janelia fluor 549) and acceptor fluorophore (Atto643). The left panel illustrates the fluorophore constructions, indicating the presence or absence of a polyproline linker (shown as a helix) on the three peptides. (i) The scatter plot of the intensity of peptides across the 560 and 640 channel is shown for each of the three peptides. The peptide with no Pro (30) linker (top row) had only <5% of colocalized spots, while high colocalization (67%) was observed when fluorophores were constructed with a Pro (30) linker (bottom row). (ii) The FRET efficiency across the three peptides for each of the individual peptide measurements is shown. The stoichiometry value for every individual peptide measurement is the ratio of donor and acceptor fluorophore after normalization of intensity and cross-talk across the channels. The spacing of fluorophores through the construction of a Pro (30) linker reduced FRET efficiency to less than 10% (shown in the bottom row).

FIGS. 16A-16D depict how PEG/polyproline linkers (polymers or “Promers”) mitigate dye-dye interactions on peptides with multiple fluorophores. FIG. 16A illustrates a polymer (or “Promer”) design and structure, with a 30-unit proline repeat flanked by a fluorophore (R₂) linked to a lysine residue, and a DBCO reactive moiety (R₁) linked via a flexible Glycine-Peg2-Glycine spacer (SEQ ID NO: 35). FIG. 16B depicts the intensity histogram of 59,405 partially photobleached peptides (JSP126) showing three distinct peaks, indicating the resolution of one, two, or three active Atto643 fluorophores (installed via polymers (or “Promers”) at azido-lysine residues at amino acid positions 2, 6, and 8). The additive nature of the fluorescence intensities (median values for 1, 2, and 3 dyes, respectively, are 17,838; 35,040; and 52,242 arbitrary units) demonstrates minimal quenching between the fluorophores. FIG. 16C depicts a representative TIRF micrograph (left panel) of individual JSP212 (SEQ ID NO: 8) peptides, labeled with Atto643 (FRET acceptor) and JF549 (FRET donor) dyes on polymers at the 2nd and 3rd amino acid positions, and demonstrates low FRET levels between the dyes. This composite image was made from offsetting the signal from three fluorescent channels (1-acceptor, 2-FRET, and 3-donor), shown enlarged for a single peptide molecule at right. The presence of signals in both donor and acceptor channels indicates the magnitude of FRET effect is less than 10% even on consecutive amino acids due to the polymers (or “Promers”). FIG. 16D is a scatter plot of fluorescence intensities across the donor and acceptor channels and shows that 67% of individual peptide molecules (10,385 filtered peptides) exhibit two distinct fluorophores, confirming low FRET levels when dyes are tethered by polymers (or “Promers”) of the present disclosure. Scale bar, 10 μm.

FIG. 17 depicts data to demonstrate that Edman degradation occurs at similar rates for peptides containing a fluorescent polymer (or “Promer”). There was no significant difference in Edman degradation efficiency observed between peptides with fluorophores constructed without and with polymers (JSP263 (SEQ ID NO:19), JSP274 (SEQ ID NO:34)), as indicated by the average loss of 62% of fluorescent peptides at the 3rd position.

FIGS. 18A-18D depict fluorosequencing a two color, four peptide mixture. FIG. 18A shows four fluorescently labeled peptides 1-4 (detailed in Table 6; SEQ ID NOs: 9-12) that were mixed at approximately equimolar (2 μM) concentrations, diluted by four orders of magnitude (to 200 pM), and fluorosequenced, thus collecting raw sequencing reads for 49,480 individual peptide molecules. FIG. 18B shows a representative TIRF microscope image with overlaid 561 and 647 nm channels, with data plotted on the right-hand side for the four distinct peptides labeled 1-4, each exhibiting a unique fluorosequencing profile, shown in the individual peptide micrographs for consecutive Edman cycles in the two fluorescent channels. The associated plots report sequencing read fluorescent intensities for 331, 1423, 1731, and 1012 replicate molecules, respectively. FIG. 18C depicts use of a machine learning classifier to accurately classify peptides to one of the 4 source peptides in a reference database with 50 decoy peptides. The 4,948 peptides classified with scores >0.99 were also visualized in a UMAP plot in FIG. 18D by considering their raw reads as feature vectors; the four distinct peptides are clearly separable. Also shown in FIG. 18D is the presence of peptide-2 with a missed Edman cycle, depicted in cluster (a), but which is still classified correctly. A minor population of peptides with photobleached Atto647 dye, depicted in cluster (b), was classified as peptide-1.

FIGS. 19A-19C depicts the computational workflow for inferring peptide sequences from raw fluorosequencing data. Briefly, the workflow comprises of several major parts. FIGS. 19A-19B: Building of a machine learning classifier: Using the input peptide set and experimentally determined fluorosequencing parameters, namely, Edman efficiency, photobleaching rates, dye destruction rates, dud dye rates and dye intensity distributions, possible fluorosequencing reads are simulated. With the knowledge of the source peptide, random forest is used to train and test to build a classifier. Image processing: the raw images obtained for 1000s of images across different fluorescent channels and Edman cycles are collected, aligned, filtered and fluorescence intensity reads obtained. FIG. 19C: Each fluorescent track is then classified to an input peptide with a score. Applying a score threshold, the counts of individual peptides present in the input sample are collated. Details of the protocol are given in Examples 8 and 9.

FIGS. 20A-20B depict the score distribution between decoy and input peptide set. The results of the experiment scored 49,480 fluorescent tracks to the most likely peptide, either to the 4 peptides present in the input set or the decoy peptides (see methods in Example 8). FIG. 20A presents a histogram showing the count of peptides for various classification scores. It is clear from this data that higher scores correspond to peptides from the input set. On the other hand, the decoy peptides are not highlighted. In FIG. 20B, the peptides that have been classified are evaluated on a precision/recall curve. The data suggests that 8% of the peptides (in terms of recall) had a very high precision of 99%.

FIGS. 21A-21C depict the sequencing of a target HLA-1 peptide, demonstrating a potential application of fluorosequencing to address a clinical need. FIG. 21A shows a pilot study that was conducted on a mono-allelic B-cell line (HLA A2603) to compare potential neoantigenic HLA-1 peptides inferred from genomic and transcriptomic information with direct identification through mass spectrometry. The study revealed significant disparity of HLA-1 peptides, predicted through prediction algorithms and direct measurements. Out of 1194 peptides identified from mass spectrometry, only 546 were predicted to have strong affinity, and potentially four peptides with mutations were noted. Since the sensitivity requirement of detection of these peptides are typically high, one of these peptides (JSP308) was chosen, labeled, and fluorosequenced. The experimental reads were classified against a reference database of the 37 well-observed HLA-1 peptides (observed with more than 2 peptide-spectral matches) from the larger set of 1,194 mass spectrometry-identified peptides. FIG. 21B shows a representative image of one individual peptide molecule across Edman cycles (image series) and measured fluorescence intensities of 111 replicate peptide molecules (plotted values) to illustrate accurate sequencing of the peptide. FIG. 21C illustrates a UMAP projection, plotted as in FIG. 18D. The UMAP projection shows that, despite the target peptide sequence having only three fluorophores and several similar peptides in the database (because HLA-I peptides bound by the same HLA allele share partial sequence identity, in this case at the peptide E1 position), 30% of experimental reads could still be correctly identified among those 676 reads classified with scores ≥0.7.

FIG. 22 depicts a flow chart describing polymer (or “promer”) synthesis.

FIGS. 23A-23C depict quality control results from quality checkpoints A-C. FIG. 23A: Validation of Checkpoint A. (Top) A liquid chromatogram showing the results of a test cleavage performed following the synthesis of Fmoc-Pro30-K(boc)-ONH2. (Bottom) The resulting ESI mass spectra from integrating the area under the curve from 2.5-5 minutes on the tandem mass spectra. FIG. 23B: Validation of Checkpoint B. (Top) A liquid chromatogram showing the results of a test cleavage performed following the synthesis of Fmoc-PEG2-G-Pro30-K(boc)-ONH2 after employing a triple coupling. (Bottom) The resulting ESI mass spectra from integrating the area under the curve from 2.5-5 minutes on the tandem mass spectra. This mass spectra reveals the coupling of Fmoc-PEG2-COOH goes to completion after utilizing a triple coupling. FIG. 23C: Validation of Checkpoint C. (Top) Liquid chromatography results from the coupling of Fmoc-G-COOH to the loaded resin. (Bottom) Tandem ESI mass spectra integrated from 2.5-5 minutes, suggesting the major product of synthesis to be Fmoc-G-PEG₂-G-Pro30-K—ONH₂.

FIGS. 24A-24B depicts validation of a DBCO functionalized polymer. FIG. 24A: Liquid chromatography results. FIG. 24B: Tandem ESI mass spectra.

FIGS. 25A-25B depicts validation of a Atto643 labeled DBCO-functionalized polymer. FIG. 25A: Liquid chromatography results. FIG. 25B: Tandem ESI mass spectra.

FIGS. 26A-26B depict the mitigation of dye-dye quenching by a polymer of the present disclosure. Comparing the histogram of intensity distribution of individual peptide molecules with 3 Atto647N dyes (FIG. 26A) on polymers with peptide molecules without polymers (FIG. 26B) shows the effect of polymers in reducing quenching behavior.

FIGS. 27A-27B depict the mitigation of dye-dye FRET by a polymer of the present disclosure. The FRET effect between the donor fluorophore (Alexa555) and acceptor fluorophore (Atto643) is mitigated through use of polymers. FIG. 27A shows 82% colocalization of spots across the donor and acceptor channels. In FIG. 27B, there is a missing donor signal and <5% of colocalization of spots, but signal appears in the FRET channel when there are no polymers attaching the donor fluorophore (JFX555) and acceptor fluorophore (Atto643).

FIGS. 28A-28C depict analytical data from the conjugation of NHS-DBCO to non-functionalized polyproline. FIG. 28A: Liquid chromatograph collected immediately following the cleavage event from the resin. Two major products are seen in the trace: a hydrophilic side product generated from exposing DBCO to acidic conditions, and the DBCO containing target compound. FIG. 28B: Chemdraw of the target product with an elution time of 4.887 minutes. FIG. 28C: Chemdraw of the degradation product with an elution time of 3.774 minutes.

FIGS. 29A-29C depict analytical data of a purified DBCO functionalized polyproline. FIG. 29A: MALDI-TOF/TOF mass spectra of a purified DBCO functionalized polyproline. The spectrum was gathered in reflective mode utilizing a scan range between 100-4,000 Da. These data provide evidence for the presence of deletions in a sample that appears largely homogeneous via LC/MS. FIG. 29B: Liquid chromatogram of the purified DBCO functionalized polyproline sample analyzed in FIG. 29A. FIG. 29C: Tandem-in-space ESI mass spectra of the major target peak presented in FIG. 29B. The spectra appear to be largely a single product.

FIGS. 30A-30E depict analytical data of a conjugation reaction of an NHS ester dye to a functionalized polyproline. FIG. 30A: DAD isoabsorbance plot generated from a tandem LC/MS analysis of a crude sample of DBCO-G-PEG2-G-Pro30-K—(NH2)-CONH2 reacted with Atto643-NHS. FIG. 30B: Liquid chromatograph measured at 280 nm of the crude reaction between Atto643-NHS ester and DBCO-G-PEG₂-G-Pro30-K—(NH₂)—CONH2. The side product of the DBCO degradation can be seen at a retention time of t=5.054 minutes, and target product at t=6.433 minutes. FIG. 30C: Liquid chromatograph measured at 643 nm for the purified Atto643 promer. The degradation side product is seen again at t˜5 minutes. FIG. 30D: Tandem mass spectra at t=5.000 minutes, providing evidence of degradation side product. FIG. 30E: Tandem mass spectra at t=6.400 minutes, providing evidence of target product.

### DETAILED DESCRIPTION

The instant disclosure relates to polymers that include a functional group configured to couple to a polypeptide or protein, a backbone, and an amino acid residue conjugated to a fluorophore, and methods of reducing dye-dye interactions using such polymers.

Modern diagnostics have made significant progress by measuring the levels of individual proteins, as exemplified by detecting COVID spike proteins in antibody tests (see, for example, Perkmann et al. (2021) Microbiology Spectrum 9 (1): e0024721) or quantifying PD-L1 levels to stratify patients for immunotherapy (see, for example, Kowanetz et al. (2018) PNAS 115 (43): E10119-26). However, many diseases may arise due to perturbations in the levels of multiple proteins, their modifications and their interactions, making it crucial to accurately measure them simultaneously for diagnosis or treatment. Historically, proteomics technologies use either minimal sample abundances of proteins in range of femto- to attomole ranges, e.g. in the case of typical mass spectrometry experiments (see, for example, Timp and Timp (2020) Science Advances 6 (2): eaax8978), or have low quantitative accuracy, as in the case of affinity-based methods (see, for example, Katz et al. (2022) Science Advances8 (33): eabm5164). Applications to clinical settings may benefit from technology capable of accurately measuring the abundances of multiple proteins in their various modified states with high sensitivity, while working with limited samples, such as small tissue biopsies or fine needle aspirates (see, for example, Boys et al. (2023) Proteomics 23 (7-8): 2200238).

The need for quantitative, high-sensitivity measurements of peptides and proteins from biological mixtures has recently spurred the development of new single molecule protein sequencing technologies (see, for example, Restrepo-Pérez, Joo, and Dekker (2018) Nature Nanotechnology 13 (9): 786-96; Floyd and Marcotte (2022) Annual Review of Biophysics 51 (1): 181-200; Brady and Meyer (2022) Biophysics Reviews 3 (1): 011304; Callahan et al. (2020) Trends in Biochemical Sciences 45 (1): 76-89; Alfaro et al. (2021) Nature Methods 18 (6): 604-17). These technologies are currently in various stages of development, from conceptual proposals (see, for example, Palmblad (2021) Journal of Proteome Research 20 (6): 3395-99; Egertson et al. (2021) Systems Biology. https://doi.org/10.1101/2021.10.11.463967) to demonstrations on simple peptide or protein samples (see, for example, Reed et al. (2022) Science 378 (6616): 186-92; Brinkerhoffetal. (2021) Science 374 (6574): 1509-13; Aubin-Tam et al. (2011) Cell 145 (2): 257-67; de Lannoy et al. (2021) iScience 24 (11): 103239; Borgo and Havranek (2014) Protein Science 23 (3): 312-20), but in general have not yet been applied to analyses of complex peptide mixtures. A number of challenges need to be overcome in order to generate robust and scalable technologies that may be applied to complex samples.

The instant disclosure provides modifications and improvements of a highly parallelized single molecule peptide sequencing platform known as fluorosequencing (see, for example, Swaminathan et al. (2018) Nat. Biotechnol. 36, 1076-1082). Fluorosequencing is based on the principle that determining the positions of a few, select amino acids within a peptide can be sufficient for matching the partial sequence (termed a fluorosequence) to a reference database to infer the peptide or protein identity. In some embodiments, to determine the fluorosequence for individual peptide molecules, first, peptide side chains of select amino acid types are labeled with fluorescent dyes. In some embodiments, millions of the labeled peptides are then immobilized in a flow cell and imaged using total internal reflection fluorescence (TIRF) microscopy. In some embodiments, cycles of Edman degradation are performed, which remove one N-terminal amino acid from each peptide on each cycle, and the peptides' fluorescent intensities are measured after each Edman cycle. In some embodiments, using image analysis and signal processing, the cycles corresponding to the removal of fluorescent amino acids are determined for each molecule, resulting in a fluorosequence for each molecule that may be matched to the reference database to identify the peptide. Prior publications (see, for example, Swaminathan, Boulgakov, and Marcotte (2015) PLOS Computational Biology 11 (2): e1004080; Swaminathan et al. (2018) Nat. Biotechnol. 36, 1076-1082), have introduced this concept and methodology, discussed the sources of errors, and demonstrated its feasibility for sequencing simple peptide mixtures.

The present disclosure describes, in some embodiments, methods for mitigating dye-dye interactions encountered during scaling of fluorosequencing methods. In particular, the present disclosure provides polymers and methods of using the same. The polymers of the present disclosure, also known as “tethers” or “promers”, may be heteropolymers including natural and/or unnatural amino acids to form a polypeptide chain. The polymer may be conjugated near or at the N- and/or C-termini with a “functional group”, and one or more fluorophores at opposite termini. The polymer may include an amino acid residue conjugated to the fluorophore. The polymer may be attached to the side chain of select amino acids of proteins or polypeptides via the functional group. In some embodiments, the polymers may include a reactive chemical moiety, a flexible solubilizing PEG spacer, and a 30-unit proline polymer with a fluorophore on the other end. By spacing the dyes on a biomolecule through use of these polymers, the quenching of similar fluorophores and FRET phenomena between different fluorophores may be reduced.

Installing more than one polymer on the same protein biomolecule, for example, protein or polypeptide, may mitigate dye-dye interactions between the fluorophores conjugated to the polymer, for example, compared to dye-dye interactions of identical fluorophores attached directly to the protein or polypeptide without use of the polymer.

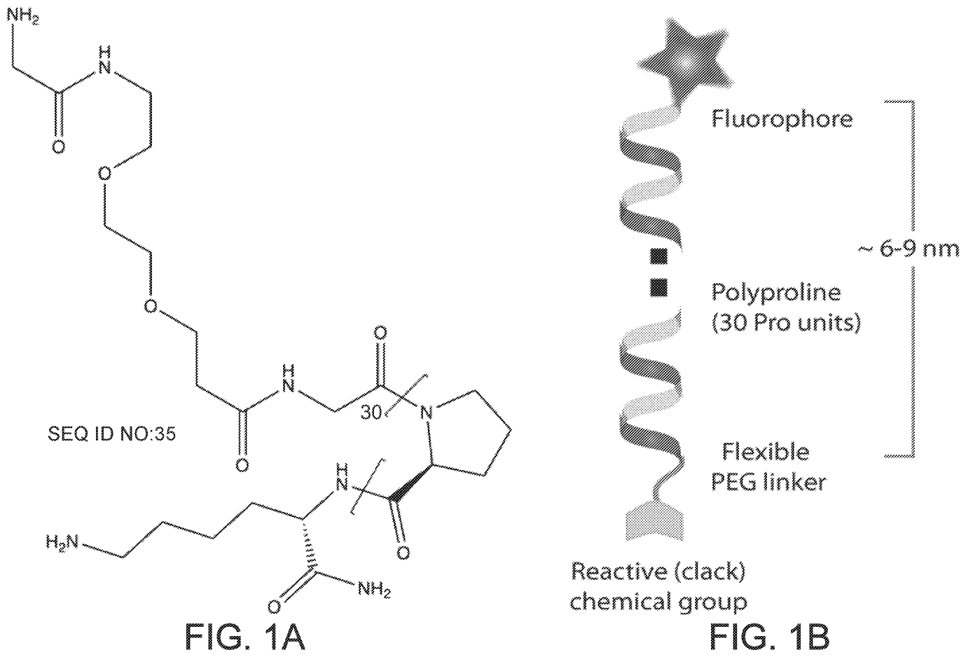

In some embodiments, a polymer may include a backbone. The backbone may include a rigid polypeptide and/or a flexible spacer. The rigid polypeptide may include a polyproline helix. The flexible spacer may include a flexible PEG spacer. The polymer may include the functional group, the flexible spacer, the rigid polypeptide and the amino acid residue conjugated to a fluorophore in amino-terminal to carboxy-terminal direction or carboxy-terminal to amino-terminal direction (FIGS. 1A-1B).

The effect of polymers on dye-dye interactions may be observed using UV-Visible spectroscopy (UV-Vis), fluorimetry or Total Internal Reflection Fluorescence Microscopy (TIRF), and other spectroscopic methods. The polymers of the present disclosure may be used in fluorosequencing, where multiple polymers may be attached to individual proteins or peptides. In turn, the sequence of the peptide may be determined with a higher accuracy due to improved measurement, modeling and/or discrimination of the fluorophore signal. The fluorosequencing may be used to study biological species and their associated macromolecules on zeptomole scales. The quantification of these species may be used for pharmaceutical, medical, zoological, scholarly, and other biological applications that involve proteomic studies of an organism.

The polymers and methods of the present disclosure may, among other benefits, provide for improved fluorosequencing of a protein or polypeptide. In some embodiments, when multiple polymers that include multiple fluorophores are attached to individual proteins or peptides, the polymers and methods of the present disclosure may provide for mitigated dye-dye interactions of the fluorophores as compared to dye-dye interactions of identical fluorophores attached directly to the protein or polypeptide. In some embodiments, the polymers and methods of the present disclosure may provide for fluorosequencing of proteins and/or polypeptides with improved accuracy and efficiency compared to fluorosequencing without use of polymers, at least in part due to mitigated dye-dye interactions when multiple polymers that include multiple fluorophores are attached to individual proteins or peptides. In some embodiments, a rigid polypeptide included in the polymer may mitigate dye-dye interactions when multiple polymers that include multiple fluorophores are attached to individual proteins or peptides, as compared to a polymer that does not include a rigid polypeptide. In some embodiments, when multiple polymers that include multiple fluorophores are attached to individual proteins or peptides, the polymers and methods of the present disclosure may provide for reduced dye quenching between the fluorophores as compared to dye quenching between identical fluorophores attached directly to the protein or polypeptide. In some embodiments, when multiple polymers that include multiple fluorophores are attached to individual proteins or peptides, the polymers and methods of the present disclosure may provide for reduced Förster resonance energy transfer (FRET) between the fluorophores as compared to FRET between identical fluorophores attached directly to the protein or polypeptide.

The present disclosure also provides, in additional aspects, improvements to the fluorosequencing workflow that facilitate scaling the technology to identify multiple peptides in mixtures. For example, fluorophores that were stable across the chemical solvent and exhibited high brightness for single molecule TIRF experiments can be used, improving Edman chemistry for greater reproducibility and efficiency, and modifying peptide-slide attachment through the azide-alkyne click reaction.

### I. Glossary

The following sections provide a detailed description of polymers that include a functional group, a backbone, and an amino acid residue conjugated to a fluorophore, and methods related to the polymers. Prior to setting forth this disclosure in more detail, it may be helpful to an understanding thereof to provide definitions of certain terms to be used herein. Additional definitions are set forth throughout this disclosure.

In the present description, the term “about” means ±20% of the indicated range, value, or structure, unless otherwise indicated.

The term “comprise” (and similar terms such as “comprising of” and “comprised of”) means the presence of the stated features, integers, steps, or components as referred to in the claims, but that it does not preclude the presence or addition of one or more other features, integers, steps, components, or groups thereof. The term “consisting essentially of” limits the scope of a claim to the specified materials or steps and those that do not materially affect the basic and novel characteristics of the claimed invention.

It should be understood that the terms “a” and “an” as used herein refer to “one or more” of the enumerated components. The use of the alternative (e.g., “or”) should be understood to mean either one, both, or any combination thereof of the alternatives, and may be used synonymously with “and/or”. As used herein, the terms “include” and “have” are used synonymously, which terms and variants thereof are intended to be construed as non-limiting or open-ended.

The word “substantially” does not exclude “completely”; e.g., a composition which is “substantially free” from Y may be completely free from Y. Where necessary, the word “substantially” may be omitted from definitions provided herein.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “analyte,” as used herein, generally refers to a substance (e.g., a molecule) whose presence or absence is measured or identified. An analyte can be a substance (e.g., molecule) for which a detectable probe may be used to identify the presence or absence of such substance. As a non-limiting example, an analyte can be a macromolecule, such as, for example, a peptide or a protein. An analyte can be part of a sample that contains or is suspected of containing other components, or can be the sole or the major component of the sample. An analyte can be a component of a whole cell or tissue, a cell or tissue extract, a fractionated lysate or a cell or tissue, or a substantially purified molecule. In some embodiments, the analyte is a peptide.

The term “biomolecule,” as used herein, generally refers to a molecule that includes a component that may be present in an organism. A biomolecule may include a molecule that is essential to a biological process. A biomolecule may include a natural molecule, or may include an unnatural molecule that includes a component of a natural molecule. In some embodiments, the biomolecule may include a peptide. In some embodiments, the biomolecule may include a protein. In some embodiments, the biomolecule may include a nucleic acid, for example, an RNA molecule, a DNA molecule, or any combination thereof. In some embodiments, the biomolecule may include a carbohydrate, lipid, fatty acid, metabolite, polyphenolic macromolecule, vitamin, hormone, or any combination thereof.

As used herein, the terms “peptide”, “polypeptide”, and “protein” and variations of these terms refer to a molecule, in particular a peptide, oligopeptide, polypeptide, or protein including fusion protein, respectively, comprising at least two amino acids joined to each other by a normal peptide bond, or by a modified peptide bond, such as for example in the cases of isosteric peptides. For example, a peptide, polypeptide, or protein may be composed of amino acids selected from the 20 amino acids defined by the genetic code, linked to each other by a normal peptide bond (“classical” polypeptide). The term peptide also includes molecules that are commonly referred to as peptides, which generally contain from about two (2) to about twenty (20) amino acids. The term peptide also includes molecules that are commonly referred to as polypeptides, which generally contain from about twenty (20) to about fifty amino acids (50). The term peptide also includes molecules that are commonly referred to as proteins, which generally contain from about fifty (50) to about three thousand (3000) amino acids. A peptide, polypeptide, or protein can be composed of L-amino acids and/or D-amino acids. A peptide, polypeptide, or protein may be synthetic, recombinant, or naturally occurring. A synthetic peptide is a peptide that is produced by artificial means in vitro. The amino acid may be a naturally occurring amino acid or a non-naturally occurring (or unnatural) amino acid (e.g., an amino acid analogue).

A peptide or polypeptide may be linear or branched. The peptide or polypeptide may include modified amino acids. The peptide or polypeptide may be interrupted by non-amino acids. A peptide or polypeptide can occur as a single chain or an associated chain. The peptide or polypeptide may have a secondary and tertiary structure (e.g., the peptide or polypeptide may be a protein comprising defined secondary, tertiary, and quaternary structures). In some examples, the peptide or polypeptide comprises at least 2, 3, 4, 5, 6, 7, 8, 9, 10, 20, 30, 40, 50, 100, 1000, 10,000, or more amino acids. The peptide or polypeptide may be a fragment of a larger polymer. In some examples, the peptide or polypeptide is a fragment of a larger peptide or polypeptide, such as a fragment of a protein.

The term “amino acid,” as used herein, generally refers to a naturally occurring or non-naturally occurring amino acid (e.g., an amino acid analogue). The non-naturally occurring (or unnatural) amino acid may be an engineered or synthesized amino acid. An amino acid may contain a “side chain”, which may differentiate amino acid types from one another.

The terms “amino acid sequence,” “peptide sequence,” and “polypeptide sequence,” as used herein, generally refer to a sequence of at least two amino acids or amino acid analogs that are covalently linked (e.g., by a peptide (amide) bond or an analog of a peptide bond). A peptide sequence may refer to a complete sequence or a portion of a sequence. For example, a peptide sequence may contain gaps, positions with unknown identities, or positions that can accommodate distinct species.

As used herein, the term “side chain” generally refers to a structure attached to an alpha carbon (attaching an amine and a carboxylic acid group of an amino acid) that may be unique to each type of amino acid. A side chain may have a certain shape, size, charge, reactivity, or a combination thereof. A side chain may contain a basic moiety (e.g., the guanidino group in arginine), an acidic moiety (e.g., the carboxylic acid in aspartic acid), a polar moiety (e.g., the hydroxyl groups in serine, threonine, and tyrosine), a hydrophobic moiety (e.g., the alkyl groups in leucine, isoleucine, alanine, and valine), or any combination thereof. In some cases, an amino acid contains more than one side chain. The side chain may be or include hydrogen, an alkyl group, a hydroxyl group, an aryl group, a heteroaryl group, a carboxylic acid, an amide, an amine, a guanidine, a thiol, a thioether, a selenol, or any combination thereof. In some instances, the side chain is a hydrogen (an amino acid with a hydrogen side chain may be, e.g., glycine).

The term “cleavable unit,” as used herein, generally refers to a moiety of a molecule that can be used to split or dissociate the molecule into two or more other molecules. A cleavable unit may be split under cleavage conditions. Non-limiting examples of cleavage conditions include use of: enzymes, nucleophilic or basic reagents, reducing agents, photo-irradiation, electrophilic or acidic reagents, organometallic or metal reagents, and oxidizing reagents.

The term “sample,” as used herein, generally refers to a chemical or biological sample containing or suspected of containing a peptide. For example, a sample can be a biological sample containing one or more peptides. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is derived from a homogenized tissue sample (e.g., brain homogenate, liver homogenate, kidney homogenate). In some embodiments, the sample is taken from a specific type of cell (e.g., neuronal cell, muscle cell, liver cell, kidney cell). The sample may be acquired from a diseased cell or tissue (e.g., a tumor cell, a necrotic cell). In some examples, the sample is from a disease-associated inclusion (e.g., a plaque, a biofilm, a tumor, a non-cancerous growth). In some examples, the sample is obtained from a cell-free bodily fluid, such as whole blood, saliva, or urine. In some examples, the sample can include circulating tumor cells. In some examples, the sample is an environmental sample (e.g., soil, waste, ambient air), industrial sample (e.g., samples from any industrial processes), and food samples (e.g., dairy products, vegetable products, and meat products). The sample may be processed prior to loading into a microfluidic device. For example, the sample may be processed to purify the peptides and/or to include reagents.

As used herein, the term “support” generally refers to an entity to which a substance (e.g., molecular construct) can be immobilized. The solid may be a solid or semi-solid (e.g., gel) support. As a non-limiting example, a support may be a bead, a polymer matrix, an array, a microscopic slide, a glass surface, a plastic surface, a transparent surface, a metallic surface, a magnetic surface, a multi-well plate, a nanoparticle, a microparticle, a lantern, or a functionalized surface. The support may be planar. As an alternative, the support may be non-planar, such as including one or more wells. A bead can be, for example, a marble, a polymer bead (e.g., a polysaccharide bead, a cellulose bead, a synthetic polymer bead, a natural polymer bead), a silica bead, a functionalized bead, an activated bead, a barcoded bead, a labeled bead, a PCA bead, a magnetic bead, or a combination thereof. A bead may be functionalized with a functional motif. Some non-limiting examples of functional motifs include a capture reagent (e.g., pyridinecarboxyaldehyde (PCA)), a biotin, a streptavidin, a strep-tag II, a linker, or a functional motif that can react with a molecule (e.g., an aldehyde, a phosphate, a silicate, an ester, an acid, an amide, an alkyne, an azide, or an aldehyde dithiolane. The functional motif may couple specifically to an N-terminus or a C-terminus of a peptide. The functional motif may couple specifically to an amino acid side chain. The functional motif may couple to a side chain of an amino acid (e.g., the acid of a glutamate or aspartate, the thiol of a cysteine, the amine of a lysine, or the amide of a glutamine, or asparagine). The functional motif may couple specifically to a reactive group on a particular species, such as a label. In some examples of functionalized beads, the functional motif can be reversibly coupled and cleaved. A functional motif can also irreversibly couple to a molecule. The functional motif may also be part of a polymer of the present disclosure.

As used herein, the term “Edman degradation” generally refers to a method of removing an amino acid from the N-terminal end of a peptide using an isothiocyanate (e.g., phenyl isothiocyanate). Edman degradation may be coupled with various peptide sequencing and analysis methods. Edman degradation may be performed sequentially.

As used herein, the term “array” generally refers to a population of sites. Such populations of sites can be differentiated from one another according to relative location. Different molecules that are at different sites of an array can be differentiated from each other according to the locations of the sites in the array. An individual site of an array can include one or more molecules of a particular type. For example, a site can include a single peptide having a particular sequence or a site can include several peptides having the same sequence. The sites of an array can be different features located on the same substrate. Such features may include, without limitation, wells in a substrate, beads (or other particles) in or on a substrate, projections from a substrate, ridges on a substrate or channels in a substrate. The sites of an array can be separate substrates each bearing at least one molecule. Different molecules attached to separate substrates can be identified according to the locations of the substrates on a surface to which the substrates are associated or according to the locations of the substrates in a liquid or gel. Such different molecules may have the same or different sequences. An array may include one or more wells, and a well of the one or more wells may have one or more beads. As an alternative, the array may be a planar surface having, for example, a molecule immobilized thereon, or, as another example, one or more beads immobilized thereon.

As used herein, the term “label” generally refers to a molecular or macromolecular construct that can couple to a reactive group. As used herein, the term “reactive group” generally refers to a to a group of atoms that exhibits a characteristic reactivity. The label may comprise at least one reactive group (e.g., a first reactive group and a second reactive group). The at least one reactive group may be configured to couple to a peptide. The at least one reactive group may be configured to couple to a support. The at least one reactive group may be configured to couple to a reporter moiety. A label may provide a measurable signal. In some embodiments, a label may be a “polymer”, “promer”, or “tether” of the present disclosure. In some embodiments, a functional group in a polymer may be a reactive group.

The term “recombinant”, as used herein (e.g., a recombinant antibody, a recombinant protein, a recombinant nucleic acid, etc.), refers to any molecule (antibody, protein, nucleic acid, siRNA, etc.) that is prepared, expressed, created, or isolated by recombinant means, and which is not naturally occurring. As used herein, the terms “nucleic acid”, “nucleic acid molecule,” and “polynucleotide” are used interchangeably and are intended to include DNA molecules and RNA molecules. A nucleic acid molecule may be single-stranded or double-stranded.

As used herein, the term “sequence variant” refers to any sequence having one or more alterations in comparison to a reference sequence, whereby a reference sequence is any of the sequences listed in the sequence listing, i.e., SEQ ID NO: 1 to SEQ ID NO:48 Thus, the term “sequence variant” includes nucleotide sequence variants and amino acid sequence variants. For a sequence variant in the context of a nucleotide sequence, the reference sequence is also a nucleotide sequence, whereas for a sequence variant in the context of an amino acid sequence, the reference sequence is also an amino acid sequence. A “sequence variant” as used herein is at least 80%, at least 85%, at least 90%, at least 95%, at least 98%, or at least 99% identical to the reference sequence. Sequence identity is usually calculated with regard to the full length of the reference sequence (i.e., the sequence recited in the application), unless otherwise specified. Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=1 1 and gap extension penalty=1]. A “sequence variant” in the context of an amino acid sequence has an altered sequence in which one or more of the amino acids is deleted, substituted or inserted in comparison to the reference amino acid sequence. As a result of the alterations, such a sequence variant has an amino acid sequence which is at least 80%, at least 85%, at least 90%, at least 95%, at least 98%, or at least 99% identical to the reference amino acid sequence. For example, per 100 amino acids of the reference sequence a variant sequence having no more than 10 alterations, i.e., any combination of deletions, insertions, or substitutions, is “at least 90% identical” to the reference sequence.

While it is possible to have non-conservative amino acid substitutions, in certain embodiments, the substitutions are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, and isoleucine, with another; substitution of one hydoxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine, and histidine, with another; and replacement of one small amino acid, e.g., alanine, serine, threonine, methionine, and glycine, with another.

The term “reporter moiety,” as used herein, generally refers to an agent that generates a measurable signal. Such a signal may include, but is not limited to, fluorescence (e.g., a dye), visible light, motion (e.g., a mass tag), radiation, or a nucleic acid sequence (e.g., a barcode). Such a signal may include, but is not limited to, fluorescence, phosphorescence, or, radiation. Such signal may be light (or electromagnetic radiation). The light may include a frequency or frequency distribution in the visible portion of the electromagnetic spectrum. For example, the light may be infrared or ultraviolet light. The signal may be an electrostatic, a conductive, or an impedance signal. The signal may be a charge. In some embodiments, a reporter moiety may be a fluorophore. A reporter moiety may be included in a “polymer” or “tether” of the present disclosure.

As used herein, the term “fluorescence” refers to the emission of visible light by a substance that has absorbed light of a different wavelength. In some embodiments, fluorescence provides a non-destructive means of tracking and/or analyzing biological molecules based on the fluorescent emission at a specific wavelength. Proteins (including antibodies), peptides, nucleic acid, oligonucleotides (including single stranded and double stranded primers) may be “labeled” with a variety of extrinsic fluorescent molecules referred to as fluorophores. Isothiocyanate derivatives of fluorescein, such as carboxyfluorescein, are an example of fluorophores that may be conjugated to proteins (such as antibodies for immunohistochemistry), peptides, or nucleic acids. In some embodiments, fluorescein may be conjugated to nucleoside triphosphates and incorporated into nucleic acid probes (such as “fluorescent-conjugated primers”) for in situ hybridization. In some embodiments, a molecule that is conjugated to carboxyfluorescein is referred to as “FAM-labeled”. In some embodiments, a protein or polypeptide may be labeled with a polymer of the present disclosure that may include a fluorophore.

As used herein, the term “conjugated” generally refers to at least two molecules, or moieties, being linked together. The molecules or moieties may be linked together by a chemical bond.

As used herein, the term “dye-dye interaction” refers to any molecular interaction between at least two dye molecules, or fluorophores. In some embodiments, a dye-dye interaction may be electrostatic. In some embodiments, a dye-dye interaction may be hydrophilic or hydrophobic. In some embodiments, a dye-dye interaction may include dye quenching or quenching. In certain embodiments, a dye-dye interaction may include Förster resonance energy transfer or FRET. In some embodiments, a dye-dye interaction may cause a change in fluorescence of a dye or fluorophore. In some embodiments, a dye-dye interaction my cause a decrease in fluorescence of a dye or fluorophore. In some embodiments, a dye-dye interaction my cause a complete loss of fluorescence of a dye or fluorophore.

As used herein, the terms “dye quenching” or “quenching” refer to any process that decreases the fluorescent intensity of a molecule, substance, or fluorophore. Quenching may result from processes such as excited state reactions, energy transfer, complex-formation and collisions. Thus, in some embodiments, quenching may depend on pressure and temperature. Examples of chemical quenchers include molecular oxygen, iodide, bromide, chloride, amines, succinimide, dichloroacetamide, dimethylformamide, pyridinium hydrochloride, imidazolium hydrochloride, methionine, Eu³⁺, Ag⁺, Cs⁺, purines, pyrimidines, N-methylnicotinamide and N-alkyl pyridinium, picolinium salts and acrylamide. Many dyes, or fluorophores, undergo self-quenching, which May decrease the brightness of protein-dye conjugates for fluorescence microscopy. Mechanisms of quenching include Förster resonance energy transfer (FRET), collisional energy transfer or Dexter energy transfer, static quenching, collisional quenching, and excited state complex or exciplex formation.

As used herein, the terms “Förster resonance energy transfer” or “FRET” refer to a mechanism describing energy transfer between two light-sensitive molecules, or chromophores. A donor chromophore, initially in its electronic excited state, may transfer energy to an acceptor chromophore through nonradiative dipole-dipole coupling. As the efficiency of this energy transfer may be inversely proportional to the sixth power of the distance between donor and acceptor, FRET is sensitive to small changes in distance. Thus, measurements of FRET efficiency may be used to determine if two fluorophores are within a certain distance of each other. The efficiency of FRET may depend on physical parameters including the distance between the donor and the acceptor (which may be in the range of 1-10 nm), the spectral overlap of the donor emission spectrum and the acceptor absorption spectrum, and the relative orientation of the donor emission dipole moment and the acceptor absorption dipole moment. In some embodiments, a chromophore may be a fluorophore. In some embodiments, FRET may be a dye-dye interaction. In certain embodiments, FRET may occur between two fluorophores.

Single molecule peptide sequencing may be used in various applications, such as, for example, protein engineering, organism engineering, and systems biology. Providing single molecule protein sequencing platforms with increased speed, accuracy, versatility, and ease of use may accelerate research across a broad range of biological and chemical disciplines. Among the challenges associated with single molecule peptide sequencing are, for example, high user input requirements, inability to handle subject peptide complexity or modifications (such as post-translational modifications), and speed and ease of use.

As used herein, sequencing of peptides “at the single molecule level” refers to amino acid sequence information obtained from individual (i.e., single) peptide molecules in a mixture of diverse peptide molecules. Peptide sequence information may be obtained from a peptide molecule or from one or more portions of the peptide molecule. Peptide sequencing may provide complete or partial amino acid sequence information for a peptide sequence or a portion of a peptide sequence. At least a portion of the peptide sequence may be determined at the single molecule level. In some cases, partial amino acid sequence information, including for example, the relative positions of a specific type of amino acid (e.g., lysine) within a peptide or portion of a peptide, may be sufficient to uniquely identify an individual peptide molecule. For example, a pattern of amino acids, such as, for example, X-X-X-Lys-X-X-X-X-Lys-X-Lys, which indicates the distribution of lysine molecules within an individual peptide molecule, may be searched against a known proteome of a given organism to identify the individual peptide molecule. Such information may be used to identify a macromolecule (e.g., protein) from which the peptide was derived, and may preclude the need to identify all amino acids of the peptide.

Peptide sequencing may be used to acquire information (including, for example, amino acid sequence information) from individual peptide molecules in a mixture of diverse peptide molecules. A method of the present disclosure may include detecting a reporter moiety coupled to amino acids of a peptide or a plurality of peptides immobilized on a solid surface (including, for example, a glass slide, or a glass slide whose surface has been chemically modified, a plastic slide, a multi-well plate, a cassette). In some cases, the detecting comprises optical (e.g., fluorescence) detection. In some cases, the reporter moiety comprises a fluorophore. In some cases, the reporter moiety comprises a plurality of amino acid-type specific labels coupled to a plurality of types of amino acids of the peptide or plurality of peptides. In some cases, the detecting comprises single-molecule (e.g., single peptide) sensitivity. In some embodiments, a method of the present disclosure may include detecting a fluorophore included in a polymer coupled to a protein or peptide.

As used herein, “single molecule resolution” refers to the ability to acquire data (including, for example, amino acid sequence information) from individual peptide molecules in a mixture of diverse peptide molecules. In one non-limiting example, the mixture of diverse peptide molecules may be immobilized on a solid surface (including for example, a glass slide, or a glass slide whose surface has been chemically modified).

In one embodiment, this may include the ability to simultaneously record the fluorescent intensity of multiple individual (i.e., single) peptide molecules distributed across the glass surface. Numerous commercially available optical devices can be applied in this manner. For example, conventional microscopes equipped with total internal reflection illumination and intensified charge-couple device (CCD) detectors may be adapted for sequencing methods disclosed herein. A high sensitivity CCD camera may be configured to simultaneously record the fluorescence intensity of multiple individual (e.g., single) peptide molecules distributed across a surface, and may be coupled to an image splitter to facilitate the simultaneous collection of multiple, distinct images (e.g., a first image comprising light of a first wavelength and a second image comprising light of a second wavelength). Using a motorized microscope stage with automated focus control to image multiple stage positions in the flow cell may allow thousands, tens of thousands, hundreds of thousands, millions, or more individual single peptides to be analyzed (e.g., sequenced) in a single experiment.

As used herein, the term “collective signal” refers to the combined signal that results from the first and second labels attached to an individual peptide molecule. In some embodiments, the labels may be polymers. As used herein, the term “subset” refers to the N-terminal amino acid residue of an individual peptide molecule. A “subset” of individual peptide molecules with an N-terminal lysine residue is distinguished from a “subset” of individual peptide molecules with an N-terminal residue that is not lysine.

### II. Polymers

In some embodiments, the present disclosure provides a polymer. The polymer may be used in fluorosequencing to mitigate dye-dye interactions. As used herein, the term “polymer” may also refer to a “promer”, or “tether” of the present disclosure.

In some embodiments, the polymer may be synthesized using a solid-phase peptide synthesizer.

In some embodiments, the polymer may include a backbone. As used herein, the term “backbone” refers to a polymer backbone. In one non-limiting example, the backbone is the main chain of a polymer. A backbone may include an organic polymer, an inorganic polymer, a biopolymer, or any combination thereof. In some embodiments, the polymer of the present disclosure may include a functional group, a backbone, and an amino acid residue conjugated to a fluorophore.

In some embodiments, the backbone may include a rigid polymer. In some embodiments, the backbone may include a flexible polymer. In some embodiments, the backbone may include both a flexible and a rigid polymer. In some embodiments, the backbone may include a homopolymer. In some embodiments, the backbone may include a heteropolymer. The backbone of a polymer may include one or more monomer subunits. In some embodiments, the backbone may include one or more natural amino acids. In some embodiments, the backbone may include one or more unnatural amino acids. In some embodiments, the backbone may include a peptide.

In some embodiments, the backbone may include a rigid polypeptide. In some embodiments, the backbone may include a rigid polypeptide that includes at least ten amino acid residues.

In some embodiments, the backbone may include a flexible spacer. In some embodiments, the polymer may include the functional group, the flexible spacer, the rigid polypeptide and the amino acid residue conjugated to a fluorophore. In some embodiments, the polymer may include the functional group, the flexible spacer, the rigid polypeptide and the amino acid residue conjugated to a fluorophore in amino-terminal to carboxy-terminal direction. In some embodiments, the polymer may include the functional group, the flexible spacer, the rigid polypeptide, and the amino acid residue conjugated to a fluorophore in carboxy-terminal to amino-terminal direction.

In some embodiments, the polymer may include one or more monomer subunits, which may be in the backbone. In some embodiments, the monomer subunit may be an amino acid residue. In some embodiments, the monomer subunit may be ethylene glycol. In some embodiments, rigidity may be established through a single monomer subunit. In some embodiments, rigidity may be established from several residues along the polymer. In some embodiments, the monomer subunit may be a proline residue, a proline residue derivative, a natural amino acid, an unnatural amino acid, a polysarcosine, a polyalanine a copolymer, or any combination thereof.

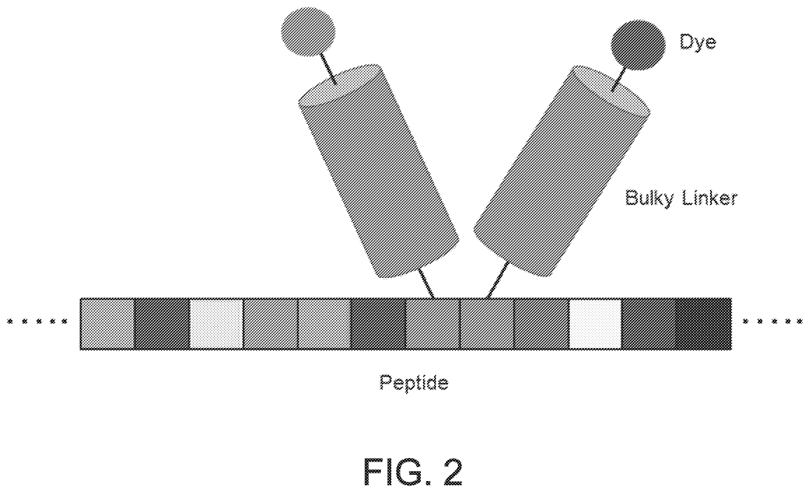

In some embodiments, rigidity may be introduced into a polymer through a bottle-brush polymer design. This design may include one or more monomer subunits along the polymer, whose side chain may introduce a significant amount of steric bulk, electrostatic repulsion, or other interaction that may increase the distance between the polymers or any of their associated residues while conjugated to a single protein or polypeptide. An example of a bottle brush polymer is shown in FIG. 2.

### a) Rigid Polypeptide

In some embodiments, the backbone may include a rigid polypeptide. As used herein, the term “rigid polypeptide” refers to a polypeptide chain that has a high conformational rigidity. In some embodiments, use of a rigid polypeptide may provide increased mitigation of dye-dye interactions between multiple fluorophores included in different polymers, as compared to identical fluorophores included in polymers that do not include a rigid polypeptide. In particular embodiments, the rigid polypeptide may include one or more proline residues, proline residue derivatives, or a combination thereof. In some embodiments, the rigid polypeptide may include a polyproline. In some embodiments, the rigid polypeptide may include a natural amino acid residue, an unnatural amino acid residue, or a combination thereof. In some embodiments, the rigid polypeptide may include a monomer subunit that is not an amino acid residue.

In some embodiments, the rigid polypeptide may include at least ten amino acid residues, wherein at least one amino acid residue of the at least ten amino acid residues may be a natural amino acid residue. In some embodiments, the natural amino acid reside may be proline. In some embodiments, the rigid polypeptide may include at least ten amino acid residues, wherein at least one amino acid residue of the at least ten amino acid residues may be an unnatural amino acid residue. In some embodiments, the unnatural amino acid residue may be a proline residue derivative.

Proline is a natural amino acid with a cyclic N-terminus. When a peptide bond is formed with proline's N-terminus, an sp₂ hybridized nitrogen arises from available resonance structures. In some embodiments, this hybridization state may lead to a trigonal planar molecular geometry at the N-terminus. In some embodiments, repeating proline monomers, otherwise known as polyproline, may form an α-helical secondary structure, which may form either a type-I helix (PPI, pitch per residue of about 1.90 Å) or type-II helix (PPII, pitch per residue of about 3.20 Å). In some embodiments, a polyproline may reduce dye-dye interactions between polymers through a combination of its rigid polymeric structure and/or its length.

Polyproline may be found as either trans or cis isomers of the peptide bond and is generally abbreviated as PPII and PPI, respectively. PPI may correspond to a contraction of the helix reducing length by roughly 40% as compared to PPII (for example, 1.90 Å and 3.20 Å for PPI and PPII, respectively, per amino acid residue). These isomers may be differentiated using circular dichroism (CD) and their formation in H₂O compared. 1-Propanol has been well characterized as the following (see, for example, Polymer Bulletin 53, 109-115 (2005)):
PPI—strong negative band at 199 nm & strong positive band at 215 nm
PPII—strong negative band at 205 nm & weak positive band at 229 nm

Isomerization between PPII and PPI may occur slowly in solutions of 1-Propanol (for example, over 14 days) and MeOH (for example, over 21 days) for Pro13. Notably, PPI although stabilized in 1-Propanol, Pro10 may denature to PPII with increasing temperature (see, for example, Protein Science (2006), 15:74-8). PPI may also be destabilized by the inclusion of electron withdrawing groups (see, for example, Protein Science (2006), 15:74-8). Solubilizing modifications may be needed to dissolve polyproline in methanol, and the rate of isomerization may increase with the number of proline residues. Notably PPII may be stable in H₂O over a 5-45° C. temperature range (see, for example, Polymer Bulletin 53, 109-115 (2005)).

The length of polyproline chains may be predicated for short sequences (for example, 1-10 amino acid residues) but longer sequences may not conform to a length of n*3.20 Å (where n=the number of amino acid residues) for PPII. These length differences may be observed when performing molecular ruler experiments using polyproline, as longer sequences may produce ruler lengths inconsistent with the expected length of a rigid proline rod. The length of long polyproline sequences has been approximated using molecular dynamics calculations and careful molecular ruler measurements (see, for example, PNAS (2005) vol. 102 no. 8 2757). Bending of the rod was found to be significant with a Pro₃₀ sequence occupying a mean length of ˜80 Å and that was capable of bending to 60 Å. A FRET efficiency of ˜18% for an Alexa Fluor® 488 and Alexa Fluor® 594 pair (which have a 58.9 Å Förster radius) using a Pro 33 ruler has been measured (see, for example, PNAS (2005) vol. 102 no. 8 2757). These results are in agreement with DNA molecular ruler data showing˜15% FRET efficiency obtained by placing an Atto550 & Atto647N pair (which have a 65.5 Å Förster radius) 85 Å (corresponding to 23 base pairs) apart on a rigid DNA double helix (see, Nature Methods, Vol. 15, 2018, pages 669-676). In some embodiments, a polymer including a polyproline that includes 30 proline residues may have a length of about 80 Å, or about 8 nm.

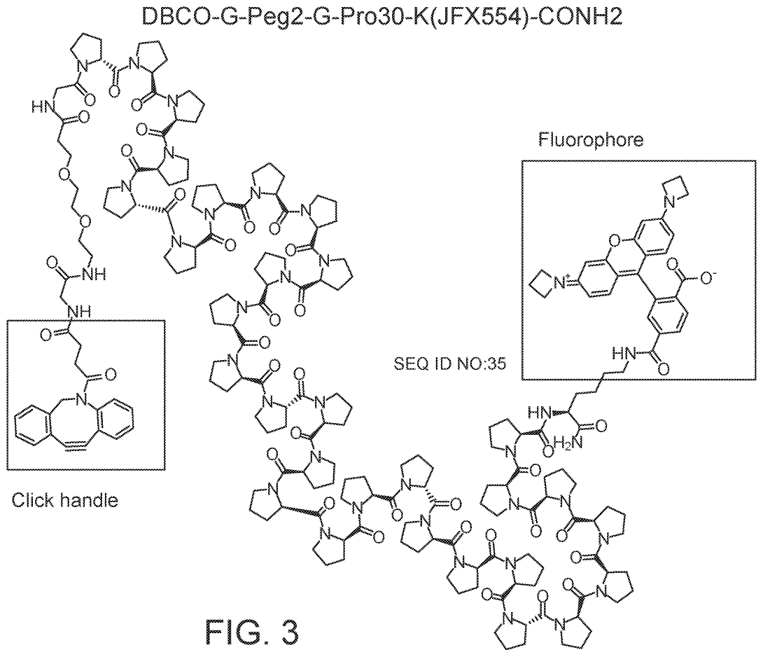

In some embodiments, a polymer may include ten or more proline monomers as a central backbone. In some embodiments, the polymer may include a majority of monomer subunits that are proline residues. In some embodiments, the polymer may include a majority of monomer subunits that are amino acid residues that are not proline. An example of a polymer is shown in FIG. 3. In some embodiments, the C-terminal end of a polyproline in the polymer may be synthesized starting from a lysine amino acid residue. In some embodiments, the lysine amino acid residue may provide an amine functional group for coupling to a fluorophore.

In some embodiments, the rigid polypeptide includes at least ten proline residues, proline residue derivatives, or a combination thereof. In some embodiments, the rigid polypeptide includes from about ten to about 40 proline residues, proline residue derivatives, or a combination thereof. In some embodiments, the rigid polypeptide includes at least 14 proline residues, proline residue derivatives, or a combination thereof. In some embodiments, the rigid polypeptide includes at least 25 proline residues, proline residue derivatives, or a combination thereof. In some embodiments, the rigid polypeptide includes at least 30 proline residues, proline residue derivatives, or a combination thereof.

In some embodiments, the rigid polypeptide includes at least ten proline residues. In some embodiments, the rigid polypeptide includes from about ten to about 40 proline residues. In some embodiments, the rigid polypeptide includes at least 14 proline residues. In some embodiments, the rigid polypeptide includes at least 25 proline residues. In some embodiments, the rigid polypeptide includes at least 30 proline residues.

In some embodiments, the rigid polypeptide comprises or consists of 30 proline residues, proline residue derivatives, or a combination thereof.

In some embodiments, the rigid polypeptide comprises or consists of 30 proline residues.

In some embodiments, the rigid poly peptide comprises a polyproline of between about ten and about 40 consecutive proline residues. In some embodiments, the rigid polypeptide comprises a polyproline of at least ten, at least 14, at least 25, at least 30, or 30 consecutive proline residues.

In some embodiments, the length of the polymer may be proportional to the number of monomer subunits included in the polymer. In some embodiments, the length of the rigid polypeptide may be proportional to the number of monomer subunits included in the rigid polypeptide. In some embodiments, a repeat of 30 monomer subunits may result in a length of the polymer of from about 6 nm to about 9 nm. In some embodiments, the length of the polymer may vary based on the helix type of a polyproline. In some embodiments, the length of the polymer may vary based on the solvent. In some embodiments, a polymer of from about 6 nm to about 9 nm that includes 30 proline amino acid residues may be used to mitigate a dye-dye interaction between fluorophores on different polymers, for example dye quenching or FRET. In some embodiments, longer polymer chains may be synthesized.

In some embodiments, the rigid polypeptide has a length from about 2 nm to about 12 nm. In some embodiments, the rigid polypeptide has a length from about 6 nm to about 9 nm. In some embodiments, the rigid polypeptide has a length from about 7.5 nm to about 8.5 nm. In some embodiments, the rigid polypeptide has a length of about 8 nm.

In some embodiments, a proline residue derivative may be hydroxyproline. In some embodiments, the proline residue derivative may be methylproline, fluoroproline, N-methylproline, nitroproline, acetylproline, benzylproline, carboxyethylproline, halogenated proline, for example, chloroproline, bromoproline, or iodoproline, aminoethylproline, phosphorylated proline, glycosylated proline, or thiolated proline.

### b) Flexible Spacer

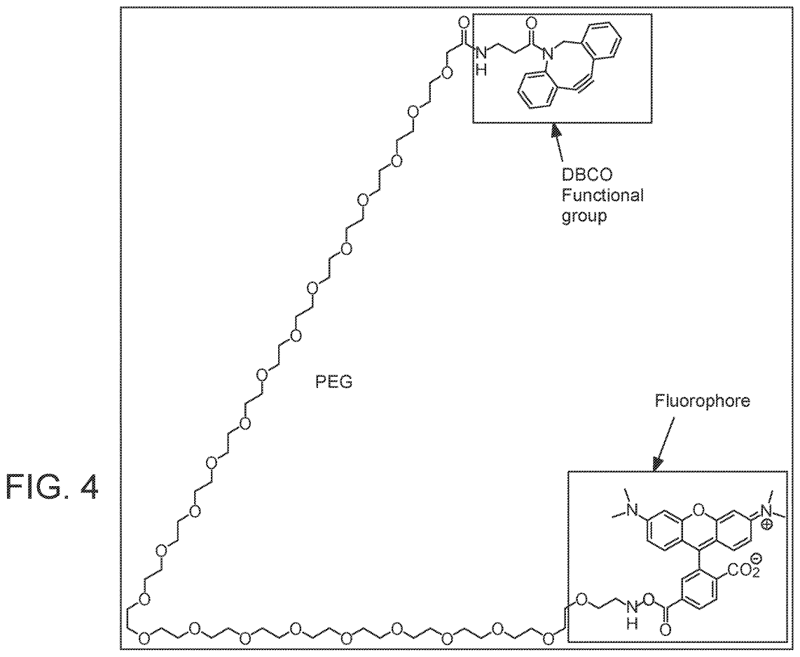

In some embodiments, the backbone may a flexible spacer. As used herein, the term “flexible spacer” refers to a monomer or polymer that that has a high conformational flexibility. In some embodiments, the flexible spacer may include polyethylene glycol (PEG). Polyethylene glycol (PEG) is a polymer molecule where the monomer subunit is an ethylene glycol. In some embodiments, a PEG chain may function as a flexible polymer body in an aqueous solution. In some embodiments, the flexible spacer may include 1-23 monomer subunits of ethylene glycol. In some embodiments, one or more flexible spacers may be included along the same backbone using solid-phase peptide synthesis process. An example of flexible spacer including a PEG polymer with 23 monomer PEG subunits is shown in FIG. 4.

In some embodiments, the flexible spacer may include an alkyl chain. In some embodiments, the alkyl chain may include 6-aminohexanoic acid, 12-aminododecanoic acid, or a combination thereof. In some embodiments, the alkyl chain may include repeats of 6-aminohexanoic acid, 12-aminododecanoic acid, or a combination of repeats thereof. In some embodiments, the alkyl chain may be synthesized into the polymer using a solid-phase peptide synthesizer.

In some embodiments, the flexible spacer may include a subunit that forms a rigid rod polymeric structure. In some embodiments, the flexible spacer may include sarcosine (N-methylglycine) copolymerized with alanine, serine, or a combination thereof.

In some embodiments, the flexible spacer may include (O—CH₂—CH₂)ₙ. In some embodiments, the flexible spacer may include Gly-(O—CH₂—CH₂)ₙ-Gly. In some embodiments, n may be a value between 1-23, inclusive. In some embodiments, n may be 2. In some embodiments, the flexible spacer may include (O—CH₂—CH₂)₂. In some embodiments, the flexible spacer may include Gly-(O—CH₂—CH₂)₂-Gly.

### c) Other Natural and Unnatural Amino Acids Including Charged Residues

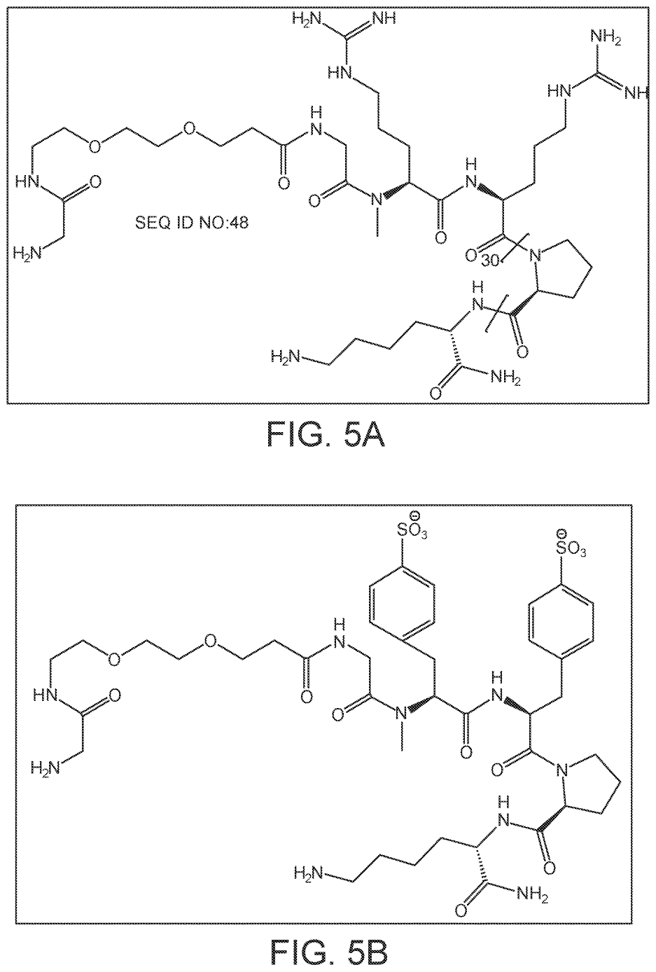

Natural and unnatural amino acids alike have a variety of side chains whose reactivity and properties may be manipulated for use in polymers. Arginine, for example, is a basic side chain whose positive charge may improve ionizability of the entire dye-polymer-functional group system via mass spectrometry. Other charged subunits that may be used in the construction of charged residues are phenylsulfonic acid and polyglutamic acid. These charged residues may also improve the chromatographic separations and detectability of polymers. Therefore, polymers may have the flexibility to be modified at any position in their sequence with a natural or unnatural amino acid that is either cationic, anionic, or zwitterionic. Introducing these charged residues may also lead to increased dispersion due to electrostatic repulsions. FIGS. 5A-5B show examples of a structure of a polymer with charged positive (arginine; FIG. 5A) or negative (phenylsulfonic acid; FIG. 5B) species.

In some embodiments, one or more natural and unnatural amino acids may be utilized to modify the properties of the polymer. In some embodiments, one or more charged amino acids may increase the rigidity of the polymer through electrostatic repulsions. In some embodiments, the chemical properties of one or more monomer subunits, which may be different monomer subunits, may influence and give rise to different properties of the polymer. In some embodiments, a moiety or monomeric subunit that is added to a polymer sequence to improve physical and/or chemical properties may be considered a modification to the polymer sequence. In some embodiments, the polymer may be modified by introducing one or more charged residues at one or both ends of the polyproline chain. In some embodiments, the charged residue may be arginine, phenylsulfonic acid, glutamic acid, or any combination thereof. In some embodiments, the polymer may be modified by introducing one or more antioxidant group. In some embodiments, the antioxidant group may be a p-nitrophenylalanine, trolox, cyclooctatetraene, or any combination thereof (see, for example, Nat. Commun. 7, 10144 (2016)). In some embodiments, the polymer may be modified by introducing one or more metal chelators. In some embodiments, the metal chelator may be an organic molecules capable of chelating lanthanides or other metals, for example, calcium, iron, and the like. In some embodiments, the polymer may include an antioxidant group. In some embodiments, the antioxidant group may be p-nitrophenylalanine, trolox, cyclooctatetraene, or any combination thereof. In some embodiments, the polymer may include a metal chelator.

In some embodiments, a polymer may include a natural amino acid residue. In some embodiments, the polymer may include a rigid polypeptide that includes at least ten amino acid residues, wherein at least one amino acid residue of the at least ten amino acid residues of the rigid polypeptide may be a natural amino acid residue. In some embodiments, the polymer may include an unnatural amino acid residue. In some embodiments, the polymer may include a rigid polypeptide that includes at least ten amino acid residues, wherein at least one amino acid residue of the at least ten amino acid residues of the rigid polypeptide may be an unnatural amino acid residue. In some embodiments, the unnatural amino acid residue may be a proline residue derivative.

In some embodiments, the polymer may include an amino acid residue that is cationic, anionic, zwitterionic, or any combination thereof. In some embodiments, the polymer may include an amino acid residue that is cationic. In some embodiments, the polymer may include an amino acid residue that is anionic. In some embodiments, the polymer may include an amino acid residue that is zwitterionic. In some embodiments, the polymer may include an arginine, an alanine, glutamic acid, or any combination thereof. In some embodiments, the polymer may include an arginine. In some embodiments, the polymer may include an alanine. In some embodiments, the polymer may include glutamic acid. In some embodiments, the polymer may include a phenylsulfonic acid, a polyglutamic acid, a polysarcosine, a polyalanine, or any combination thereof. In some embodiments, the polymer may include a phenylsulfonic acid. In some embodiments, the polymer may include a polyglutamic acid. In some embodiments, the polymer may include a polysarcosine. In some embodiments, the polymer may include a polyalanine. In some embodiments, the polymer may include a copolymer.

### d) Functional Groups

A polymer may include a functional group. As used herein, the term “functional group” generally refers to a substituent or moiety of a polymer that causes a characteristic chemical reaction. A functional group may be a reactive group that is included in a polymer of the present disclosure. The functional group may be configured to couple and/or conjugate to a polypeptide or protein. In some embodiments, the functional group may be used to conjugate the polymer to a protein or polypeptide for use in fluorosequencing.

In some embodiments, the functional group may directly conjugate with the polymer. In some embodiments, the functional group may be iodoacetamide or maleimide. In some embodiments, the iodoacetamide or maleimide may form a thioether bond with a thiol in a cysteine amino acid residue present in a protein or polypeptide. In some embodiments, the functional group may be a succinimidyl ester group. In some embodiments, the succinimidyl ester group may form an amide bond with an epsilon amine of a lysine amino acid residue in a protein or polypeptide.

In some embodiments, the functional groups may conjugate to the side chain of an amino acid through a two-step chemistry termed “click-clack”. In click-clack chemistry, the amino acid side chain maybe labeled selectively through a bifunctional molecule. In some embodiments, one half of the bifunctional molecule may react with the amino acid side chain. In some embodiments, the other half of the bifunctional molecule may include a reactive group such as an azide, a norbornene, and the like. In some embodiments, this is a “click” group. In some embodiments, the functional group of the polymer may include a “clack” group or a “click handle”. In some embodiments, the clack group may react with the click group selectively and orthogonally. In some embodiments, the clack group included in the polymer may be a DBCO group. In some embodiments, the DBCO included in the polymer may react with an azide group, which may be the click group attached to the peptide.

In some embodiments, the functional group may include a clack group. In some embodiments, a polymer may have at least one clack group (click partner) for bioconjugation at each terminus. The clack group may be conjugated at either terminus, but may be conjugated to the opposite terminus to that of the fluorophore(s). In some embodiments, the clack group may be configured to react with a click group on a peptide.

In some embodiments, a polymer may include a functional group that is DBCO, a methyltetrazine or a lipoic acid conjugated at one terminus. In some embodiments, conjugation of a DBCO, a methyltetrazine or a lipoic acid may produce a reactive end to the polymer. FIG. 3 shows a configuration of one embodiment of a polymer that includes a functional group (a “click handle”) that is a DBCO group and a fluorophore that is a JFX 554.

In some embodiments, the functional group may include a iodoacetamide, a maleimide, an amine, an azide, an alkene, an aldehyde, a ketone, a tetrazine, an alkyne, a strained alkyne, a cycloalkyne, a cyclooctyne, dibenzocyclooctyne (DBCO), a thiol, a carboxyl, a hydrazide, a dithiol, a trans-cyclooctene, a bicycloalkene, an iodobenzene, a cyanothiazole, an acene, a dithiolane, a bromane, an aminothiol, a pyrroledione, a sulfenyl chloride, a succinimidyl ester, a succinidimyl ester, methyltetrazine, lipoic acid, or any combination thereof.

In some embodiments, the functional group may be a strained alkyne.

In some embodiments, the functional group may include dibenzocyclooctyne (DBCO), methyltetrazine or lipoic acid. In some embodiments, the functional group may include dibenzocyclooctyne (DBCO). In some embodiments, the functional group may be dibenzocyclooctyne (DBCO).

### e) Fluorophores

A polymer may include a fluorophore. As used herein, the term “fluorophore” refers to a fluorescent molecule. A polymer may include multiple fluorophores. The fluorophore may be conjugated at the opposite end of the polymer to the functional group. In some embodiments, the fluorophore may be included at the C-terminus of the polymer. In some embodiments, the fluorophore may be included at the N-terminus of the polymer. In some embodiments, one, two, or more, fluorophores may be included at one terminus of the polymer. In some embodiments, one, two, or more, fluorophores may be included at one terminus of the polymer by the design and synthesis of one, two, or more, lysine residues at that terminus. In some embodiments, varying the fluorophore and the functional group may allow a diversity of polymers to be synthesized.

In some embodiments, the polymer may include an amino acid residue conjugated to a fluorophore. In some embodiments, the amino acid residue conjugated to the fluorophore may be included at the C-terminus of the polymer. In some embodiments, the amino acid residue conjugated to the fluorophore may be included at the N-terminus of the polymer. In some embodiments, the amino acid residue conjugated to the fluorophore may be a lysine residue, an azidolysine residue, or a cysteine residue. In some embodiments, the amino acid residue conjugated to the fluorophore may be a lysine residue.

In some embodiments, the fluorophore may be an Alexa Fluor® dye, an Atto dye, a Janelia Fluor® dye, a carbopyronine derivative, a Rhodamine derivative, or any combination thereof.

In some embodiments, the fluorophore may be Alexa Fluor® 405, Alexa Fluor® 448, Alexa Fluor® 555, Alexa Fluor® 594, Alexa Fluor® 647, Alexa Fluor® 680, Atto390, Atto425, Atto488, Atto495, Atto514, Atto532, Atto550, Atto643, Atto647N, Atto647, Atto655, Atto680, Atto700, AttoRho-12, (5)6-napthofluorescein, Oregon Green™ 488, Oregon Green™ 514, JFX554, 00488-NHS, 00488-Azide, 00488-Tetrazine, 00514-NHS, Janelia Fluor® 479, Janelia Fluor® 525, Janelia Fluor® 549, Janelia Fluor® 555, Janelia Fluor® 579, SF554, Texas Red, JFX 554, JFX 650, CFR 398, CFR 430, CFR 568, CFR 633, CFR 640R, CF® 680R, Sarafluor™ 488B, Sarafluor™ 650B, Rhodamine, Rhodamine 110 (5-CR110), Rhodamine 6G, Rhodamine B, carboxyrhodamine B, tetramethylrhodamine (TMR), Rhodamine 101, Rhodamine Si, fluorescein, 5-carboxyfluorescein, napthofluorescein, 6-JOE-N3, 7-hydroxycoumarin-N3, Cy3 Cy5, Cy3B, Cy5B, Cy488, Dylight™ 405, Dylight™ 488, iFluor® 710, DY350XL, DY351XL, DY360XL, DY370XL, DY376XL, DY380XL, DY396XL, DY720, Bodipy™ 493, BodipyTMFL, Bodipy™ 650, PB430 (Phoxbright 430), Pyrene-N3, Lucifer Yellow, Nanohoop 6, Nanohoop 8, Eterneon 394, Hilyte™ 405, Hilyte™ 488, Hilyte™ 647, or any combination thereof.

In some embodiments, the fluorophore may be Texas Red, Janelia Fluor® 525, Janelia Fluor® 549, Janelia Fluor® 555, Alexa Fluor® 448, Alexa Fluor® 555, Atto495, Atto643, Atto647N, Rhodamine, tetramethylrhodamine, or any combination thereof.

In some embodiments, the fluorophore may be Texas Red, Janelia Fluor® 549, Alexa Fluor® 555, Atto643, or any combination thereof.

In some embodiments, the polymer may include an amino acid residue conjugated to a fluorophore, and the polymer may include at least one additional amino acid residue, wherein the additional amino acid residue is conjugated to an additional fluorophore. In some embodiments, the additional amino acid residue conjugated to the additional fluorophore may be positioned adjacent to the amino acid residue conjugated to the fluorophore. In some embodiments, the fluorophore and the additional fluorophore may be the same fluorophore. In some embodiments, the fluorophore and the additional fluorophore may be different fluorophores.

### f) Example Polymer Sequences and Structures

In some embodiments, a polymer may include, in amino-terminal to carboxy-terminal direction: a functional group that is dibenzocyclooctyne (DBCO); a flexible spacer comprising Gly-(O—CH₂—CH₂)₂-Gly; a rigid polypeptide including at least ten, at least 14, at least 25, at least 30, or 30 consecutive proline residues; and a lysine residue conjugated to a fluorophore. In some embodiments, the flexible spacer may include PEG₂, PEG₄, an alkyl group, or any combination thereof. In some embodiments, the polymer may include in amino-terminal to carboxy-terminal direction: a functional group that is dibenzocyclooctyne (DBCO); a flexible spacer comprising Gly-(O—CH₂—CH₂)₂-Gly; a rigid polypeptide comprising 30 proline residues; and a lysine residue conjugated to a fluorophore.

In some embodiments, a polymer may include structure I, wherein R₁=DBCO and R₂=a fluorophore:

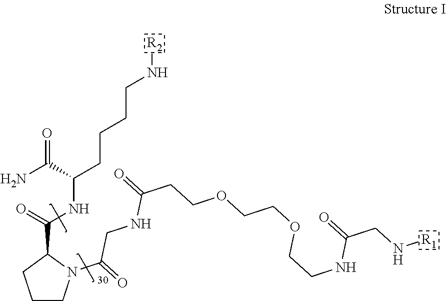

In some embodiments, the polymer may have a sequence of DBCO-Gly-(O—CH₂—CH₂)₂-Gly-Pro₃₀-Lys(fluorophore)-CONH₂. In some embodiments, a composition including at least one polymer of the present disclosure and a solvent may be provided.

### III. Proteomics

Proteomics is the large-scale study proteins present in an organism, system, or biological consortia. Proteins are quintessential to organisms, facilitating the majority of chemical and physical processes carried out by life. Accordingly, the set of proteins expressed within a cell, organism, or system often strongly reflective of health, biological state, biological activity, and physical conditions (e.g., heat stress, nutrient depletion, or stimulation). Accordingly, peptide sequencing is a tool that may be used in a variety of applications within the field of proteomics.

The present disclosure provides polymers and methods for peptide (e.g., protein) analysis (e.g., sequencing). Polymers and methods of the present disclosure may permit a peptide (e.g., protein) to be analyzed (e.g., sequenced) in a manner that provides various non-limiting benefits, such as, for example, (i) sequencing a protein or polypeptide comprising a chemically modified N-terminal amino acid (e.g., ADP-ribosylation, fluorophores, etc.), (ii) sequencing a protein or polypeptide comprising an unnatural amino acid residue (e.g., P-amino acid, peptoid, PNA, etc.), or (iii) mitigating dye-dye interactions. Peptide sequencing may be used to reveal novel biomarkers for the diagnosis of cancer and other diseases or in understanding the function of healthy cells. Peptides produced by cells or tissues may act as unique biomarkers. Enhanced detection of these biomarkers through peptide sequencing may provide earlier, more accurate diagnoses of disease.

Provided herein are polymers and methods that can be used to enhance detection of biomarkers by streamlining, enhancing, or otherwise improving the speed, efficiency, or accuracy with which a peptide can be processed or analyzed. A method of the present disclosure may be configured to analyze peptides spanning at least 2, at least 3, at least 4, at least 5, at least 6, at least 7, at least 8, at least 9, or at least 10 orders of magnitude in concentration in a sample. For example, a method of the present disclosure may permit simultaneous measurements of immunoglobulins and cytokines from human serum, peptides that are traditionally difficult to simultaneously detect due to their 7+ order of magnitude concentration differences. A method of the present disclosure may be configured to identify at least 100, at least 500, at least 1000, at least 5000, at least 10⁴, at least 5×10⁴, at least 10⁵, or at least 5×10⁵ different proteins from a sample. A method of the present disclosure may be configured to identify at least 50, at least 100, at least 150, at least 200, at least 250, at least 300, at least 400, at least 500, at least 600, at least 700, at least 800, at least 900, at least 1000, at least 1200, at least 1500, at least 1800, at least 2000, at least 2500, at least 3000, at least 3500, at least 4000, or at least 5000 types of proteins from a sample (e.g., human lung homogenate). A method of the present disclosure may be configured to simultaneously (e.g., within a single assay) identify at least 50, at least 100, at least 150, at least 200, at least 250, at least 300, at least 400, at least 500, at least 600, at least 700, at least 800, at least 900, at least 1000, at least 1200, at least 1500, at least 1800, at least 2000, at least 2500, at least 3000, at least 3500, at least 4000, or at least 5000 types of proteins from a sample (e.g., buffy coat lysate). A method of the present disclosure may be configured to identify at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the types of peptides in a biological sample (e.g., a human biological sample). For example, a method of the present disclosure may comprise coupling cysteine-specific and lysine-specific polymers to a plurality of peptides derived from a human urine sample, immobilizing about 10⁵ of the peptides to a glass slide, performing sequential rounds of polymer detection and N-terminal amino acid removal on the peptides, and comparing the identified cysteine and lysine peptide sequences against a database of known human urine peptides, thereby identifying at least 60% of the about 10⁵ peptides from the sample.

### IV. Fluorosequencing

Various aspects of the present disclosure provide polymers, compositions, and methods for peptide fluorosequencing. A fluorosequencing method disclosed herein can provide peptide sequence information at the single molecule level. For example, a fluorosequencing method may be used to identify a sequence of a peptide barcode, or to simultaneously determine sequences for a plurality of peptide barcodes. Exemplary fluorosequencing methods are provided in U.S. Pat. Nos. 9,625,469, 10,545,153, 11,105,812, 11,162,952, U.S. Patent Application Publication No. US20220163536A1, International Patent Application Publication No. WO2020072907A1, and International Patent Application Publication No. WO2021236716A2. A method consistent with the present disclosure may subject a peptide to fluorosequencing in a method including a polymer.

A characteristic feature of many fluorosequencing methods is coupling amino acid labels to a protein or polypeptide to be sequenced. A label may be an amino acid specific label (e.g., configured to couple to a specific type of amino acid or a specific set of types of amino acids). A fluorosequencing method may comprise labeling a plurality of types of amino acids with separate, amino acid type specific labels. A fluorosequencing method may comprise labeling one, two, three, four, five, six, or more different types of amino acids residues in a subject protein or polypeptide. A protein or polypeptide may comprise a label on an N-terminal amino acid, cysteine, lysine, glutamic acid, aspartic acid, tryptophan, tyrosine, serine, threonine, arginine, histidine, methionine, or any combination thereof. A protein or peptide may comprise a label on a non-canonical amino acid, such as a phosphoserine/phosphothreonine, pyroglutamic acid, hydroxyproline, azidolysine, dehydroalanine, or any combination thereof. Each of these amino acid residues may be labeled with a different label. Multiple amino acid residues may be labeled with the same label such as (i) aspartic acid and glutamic acid or (ii) serine and threonine.

In some embodiments, a label may comprise a reporter moiety. The reporter moiety may be optically detectable (e.g., fluorescent, phosphorescent, luminescent, or light absorbing). The reporter moiety may be electrochemically detectable (e.g., a redox active moiety with a characteristic oxidation or reduction potential). The reporter moiety may comprise a mass tag (e.g., for identification with mass spectrometry). A reporter moiety may identify a label to which it is attached. A plurality of labels may comprise a plurality of detectable moieties which identify labels of the plurality of labels by their type. For example, a method may comprise a plurality of types of labels configured to couple to different amino acids, each comprising a different reporter moiety that uniquely identifies the label by its type.

In the present disclosure, the label may be a polymer. The polymer may include a reporter moiety that may be a fluorophore.

The polymer may include a functional group. The functional group may be configured to couple to a polypeptide or protein. A method may comprise coupling a polymer to an amino acid of a peptide (e.g., coupling a polymer to each amino acid of a particular type), and then coupling a fluorophore or protecting group to the polymer. A method may include coupling a plurality of types of polymer including functional groups to a plurality of amino acids of a peptide, and coupling a plurality of fluorophores, protecting groups, or combinations thereof to the polymers based on their types. A method may include coupling a plurality of types of polymer to a plurality of amino acids of a peptide, wherein the plurality of types of polymer include polymers with functional groups, polymers with fluorophores (e.g., a cysteine-reactive polymer coupled to a fluorophore), polymers including both functional groups and fluorophores, or any combination thereof.

A polymer (e.g., a polymer including a functional group configured to couple to a polypeptide or protein) may reversibly or irreversibly bind to an amino acid type, and thus may be chemically (e.g., by addition of a cleavage reagent) or physically (e.g., by addition of heat or light) decoupled from a target peptide. A method may thus comprise blocking a first amino acid, labeling a second amino acid type (e.g., lysine) with a polymer, unblocking the first amino acid type, and labeling the first amino acid type with a polymer. Non-limiting examples of reversible functional groups that may be included in a polymer include silanes (e.g., trimethylsilane), acetyl groups, benzoyl groups, unsaturated pyran and furan groups, urea-forming groups, carbamate-forming groups, carbonate-forming groups, thiourea-forming groups, thiocarbamate-forming groups, thiocarbonate-forming groups, and derivatives thereof. Examples of irreversible functional groups may include alkyl groups, oxo-groups, amide-forming groups (e.g., an acyl chloride configured to convert an amine into an amide), and derivatives thereof.

Labeling specificity can be a major challenge for a fluorosequencing method. In many cases, a polymer may include reactivity toward a plurality of amino acid types. For example, some maleimide functional groups can react with cysteine, lysine, and N-terminal amines. A number of strategies may be employed to utilize or prevent such cross-reactivity. A method may comprise sequential amino acid labeling with a polymer, for example to ensure that a multi-specific polymer is added to a system after one or more amino acid types with which the multi-specific polymer is configured to couple are chemically blocked or labeled, and therefore unable to react with the multi-specific polymer.

Fluorosequencing may include removing peptides through techniques such as chemical cleavage, Edman degradation, or other forms of enzymatic cleavage following or preceding subject peptide detection. Sequential peptide removal may generate sequence or position-specific information. For example, a reduction in fluorescence following an N-terminal amino acid removal step may indicate that an amino acid labeled with a polymer, and thus that a specific type of amino acid, was disposed at a peptide N-terminal. Removal of each amino acid residue may be carried out with a variety of different techniques including Edman degradation and proteolytic cleavage. The techniques may include using Edman degradation to remove the terminal amino acid residue. Alternatively, the techniques may involve using an enzyme to remove the terminal amino acid residue. These terminal amino acid residues may be removed from either the C-terminus or the N-terminus of the peptide chain. In situations where Edman degradation is used, the amino acid residue at the N-terminus of the peptide chain is removed.

A polymer and/or fluorophore of the present disclosure may be configured to withstand conditions for removing one or more of amino acid residues from a peptide. Some non-limiting examples of potential fluorophores that may be used in the instant polymers and methods include, for example, those which emit a fluorescence signal in the red to infrared spectra such as an Alexa Fluor® dye, an Atto dye, Janelia Fluor® dye, a rhodamine dye, or other similar dyes. Examples of each of these dyes which are capable of withstanding the conditions of removing the amino acid residues include Texas Red, Alexa Fluor® 405, Rhodamine B, tetramethyl rhodamine, Janelia Fluor® 525, Janelia Fluor® 549, Janelia Fluor® 555, Alexa Fluor® 448, Alexa Fluor® 555, Atto495, Atto643, Atto647N, AttoRho12, Rhodamine, tetramethylrhodamine, and (5)6-napthofluorescein. A fluorophore may include a fluorescent peptide (e.g., green fluorescent protein or a variant thereof) or an optically detectable material, such as a carbon nanotube, a nanorod, or a quantum dot.

Peptide detection or imaging may include immobilizing the peptide on a surface. The peptide may be immobilized to the surface by coupling a peptide-derived cysteine residue, the peptide N-terminus, or the peptide C-terminus with the surface or with a reagent coupled to the surface. The peptide may be immobilized by reacting the cysteine residue with the surface or with a capture reagent coupled to the surface. Detecting the immobilized peptide may include capturing an image including the peptide. The image may include a spatial address specific to the peptide. A plurality of peptides may be detected in a single image, wherein one or more of the peptides may include a spatial address within the image. The surface may be optically transparent across the visible spectrum and/or the infrared spectrum. The surface may possess a low refractive index (e.g., a refractive index between 1.3 and 1.6). The surface may be between 10 to 50 nm thick, between 20 and 80 nm thick, between 50 and 200 nm thick, between 100 and 500 nm thick, between 200 and 800 nm thick, between 500 nm and 1 μm thick, between 1 and 5 μm thick, between 2 and 10 μm thick, between 5 and 20 μm thick, between 20 and 50 μm thick, between 50 and 200 μm thick, between 200 and 500 μm thick, or greater than 500 μm in thickness. The surface may be chemically resistant to organic solvents. The surface may be chemically resistant to strong acids such as trifluoroacetic acid or sulfuric acid. A large range of substrates (like fluoropolymers (Teflon-AF (Dupont), Cytop® (Asahi Glass, Japan)), aromatic polymers (polyxylenes (Parylene, Kisco, Calif.), polystyrene, polymethmethylacrytate) and metal surfaces (Gold coating)), coating schemes (spin-coating, dip-coating, electron beam deposition for metals, thermal vapor deposition and plasma enhanced chemical vapor deposition) and functionalization methodologies (polyallylamine grafting, use of ammonia gas in PECVD, doping of long chain end-functionalized fluoroalkanes etc.) may be used in the methods described herein as a useful surface. A 20 nm thick, optically transparent fluoropolymer surface made of Cytop® may be used in the methods described herein. The surfaces used herein may be further derivatized with a variety of fluoroalkanes that will sequester peptides for sequencing and modified targets for selection. Alternatively, an aminosilane modified surfaces may be used in the methods described herein.

The methods may comprise immobilizing the peptides on the surface of beads, resins, gels, quartz particles, glass beads, or combinations thereof. In some non-limiting examples, the methods contemplate using peptides that have been immobilized on the surface of Tentagel® beads, Tentagel® resins, or other similar beads or resins. The surface used herein may be coated with a polymer, such as polyethylene glycol. The surface may be amine functionalized or thiol functionalized.

A sequencing technique described herein may involve imaging the peptide or protein to determine the presence of one or more polymers including a fluorophore coupled to the peptide. The sequencing technique may include imaging a plurality of peptides or proteins to determine the presence of one or more polymers including a fluorophore on individual peptides from among the plurality of peptides. The sequencing technique may comprise imaging at least 10³, at least 10⁴, at least 10⁵, at least 10⁶, at least 10⁷, at least 10⁸ or more proteins or peptides (e.g., imaging a portion of a surface comprising at least 10³ to at least 10⁸ proteins or peptides). These images may be taken after each removal of an amino acid residue and thus may enable determination of the location of the specific amino acid in the peptide sequence. For example, a C-terminal immobilized peptide may comprise a sequence (from N-terminal to C-terminal) of KDDYAGGGAAGKDA (SEQ ID NO: 1, wherein ‘K’ denotes lysine, ‘D’ denotes aspartate, ‘Y’ denotes tyrosine, ‘A’ denotes alanine, and ‘G’ denotes glycine), and may comprise polymers coupled to each lysine and tyrosine residue. A first image comprising the C-terminal immobilized peptide may indicate the presence of two lysines and one tyrosine in the peptide. The N-terminal amino acid may be removed (e.g., by Edman degradation), such that a second image comprising the C-terminal immobilized peptide may indicate the presence of one lysine and one tyrosine in the peptide. This process may be repeated until a sequence of KXXYXXXXXXXKX is identified for the peptide, wherein ‘X’ indicates a non-lysine, non-tyrosine amino acid, ‘K’ indicates a lysine, and ‘Y’ indicates a tyrosine. A method of the present disclosure may identify the position of a specific amino acid in a peptide sequence. A method may be used to determine the locations of specific amino acid residues in the peptide sequence or these results may be used to determine the entire list of amino acid residues in the peptide sequence. A method may involve determining the location of one or more amino acid residues in the peptide sequence and comparing these locations to known peptide sequences, which may identify the entire list of amino acid residues in the peptide sequence. For example, identifying the positions of the lysines and cysteines in a 40 amino acid fragment of a human protein may uniquely identify the protein (e.g., only one human protein may contain the specific pattern of lysine and cysteine residues identified in the 40 amino acid fragment).

An imaging method may involve a variety of different spectrophotometric and microscopy methods, such as fluorimetry, diffuse reflectance, interferometric scattering Raman, resonance enhanced Raman, infrared absorbance, visible light absorbance, ultraviolet absorbance, and fluorescence. The fluorescent methods may employ such fluorescent techniques, such as fluorescence polarization, Forster resonance energy transfer (FRET), or time-resolved fluorescence. A spectrophotometric or microscopy method may be used to determine the presence of one or more polymers including a fluorophore coupled to a single peptide. Such imaging methods may be used to determine the presence or absence of a polymer including a fluorophore on a specific peptide sequence. After repeated cycles of removing an amino acid residue and imaging a subject peptide, the position of the labeled amino acid residue may be determined in the peptide.

### a) Use of Polymers in Fluorosequencing

In some embodiments, a polymer of the present disclosure may be used in a fluorosequencing method. In some embodiments, a polymer of the present disclosure may be present as a peptide-polymer conjugate. As used herein, the term “peptide-polymer conjugate” refers to a peptide and a polymer that are conjugated by a chemical bond, for example, a covalent bond.

In some embodiments, the peptide-polymer conjugate may include a polymer of the present disclosure and a peptide with at least one amino-acid side chain, wherein the amino-acid side chain is attached to the polymer via the functional group. In some embodiments, the peptide-polymer conjugate may include at least two polymers attached to the peptide via two different amino-acid side chains. In some embodiments, the at least two polymers of the peptide-polymer conjugate may include the same fluorophore, and dye quenching between the fluorophores may be reduced compared to dye quenching between identical fluorophores attached directly to the amino-acid side chains of the peptide. In some embodiments, the at least two polymers of the peptide-polymer conjugate may include different fluorophores, and wherein Förster resonance energy transfer (FRET) between the fluorophores may be reduced compared to FRET between identical fluorophores attached directly to the amino-acid side chains of the peptide.

In some embodiments, the present disclosure provides a method of reducing dye-dye interactions. In certain embodiments, the methods provided herein include providing a polymer according to the present disclosure; providing a biomolecule including at least two reactive groups; and attaching at least two polymers to the at least two reactive groups of the biomolecule via the functional group, wherein the two polymers comprise the same fluorophore. In some embodiments, dye-dye interactions may be reduced between identical fluorophores compared to dye-dye interactions between identical fluorophores conjugated directly to the at least two reactive groups of the biomolecule. In some embodiments, the biomolecule may include a peptide. In some embodiments, the biomolecule may include a protein. In some embodiments, the biomolecule may include a nucleic acid, for example, an RNA molecule, a DNA molecule, or any combination thereof. In some embodiments, the biomolecule may include a carbohydrate, lipid, fatty acid, metabolite, polyphenolic macromolecule, vitamin, hormone, or any combination thereof. In some embodiments, the reactive group of the biomolecule may be an amino-acid side chain.

In certain embodiments, the methods provided herein including the polymer of the present disclosure may reduce dye-dye interactions by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to dye-dye interactions between identical fluorophores conjugated directly to the at least two reactive groups of the biomolecule. In certain embodiments, reduction of dye-dye interactions when using the polymer may be measured using UV-Visible spectroscopy (UV-Vis), fluorimetry or Total Internal Reflection Fluorescence Microscopy (TIRF), and/or other spectroscopic methods.

In another aspect, the present disclosure provides a method of reducing dye quenching. In certain embodiments, the methods provided herein include providing a polymer of the present disclosure, providing a peptide with at least two amino-acid side chains, and attaching at least two polymers to the at least two amino-acid side chains via the functional group, wherein the two polymers comprise the same fluorophore; thereby reducing dye quenching between identical fluorophores compared to dye quenching between identical fluorophores conjugated directly to the at least two amino-acid side chains of the peptide. In some embodiments, the identical fluorophores may be Atto647N. In certain embodiments, dye quenching may be reduced by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to dye quenching between identical fluorophores conjugated directly to the at least two amino-acid side chains of the peptide. In certain embodiments, dye quenching may be reduced by at least about 70%. In certain embodiments, reduction of dye quenching when using the polymer may be measured using UV-Visible spectroscopy (UV-Vis), fluorimetry or Total Internal Reflection Fluorescence Microscopy (TIRF), and/or other spectroscopic methods.

In another aspect, the present disclosure provides a method of reducing Förster resonance energy transfer (FRET). In certain embodiments, the methods provided herein include providing a polymer of the present disclosure, providing a peptide with at least two amino-acid side chains; and attaching at least two polymers to the at least two amino-acid side chains via the functional group, wherein the two polymers comprise different fluorophores; thereby reducing FRET between the fluorophores compared to FRET between equivalent fluorophores conjugated directly to the at least two amino-acid side chains of the peptide. In certain embodiments, the different fluorophores may be Atto647N and Janelia Fluor® 549. In certain embodiments, FRET may be reduced by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to FRET between equivalent fluorophores conjugated directly to the at least two amino-acid side chains of the peptide. In certain embodiments, FRET may be reduced by at least about 90%. In certain embodiments, reduction of FRET when using the polymer may be measured using UV-Visible spectroscopy (UV-Vis), fluorimetry or Total Internal Reflection Fluorescence Microscopy (TIRF), and/or other spectroscopic methods.

In certain embodiments, the methods provided herein may include a) providing a plurality of peptides immobilized on a solid support, each peptide including an N-terminal amino acid and internal amino acids, the internal amino acids comprising lysine, each lysine labeled with a polymer of the present disclosure, and the polymer producing a signal for each peptide; b) treating the plurality of immobilized peptides under conditions such that each N-terminal amino acid of each peptide is removed; and c) detecting the signal for each peptide at the single molecule level. In some embodiments, in step b) the N-terminal amino acid of each peptide may be reacted with a phenyl isothiocyanate derivative. In some embodiments, the removal of the N-terminal amino acid in step b) may be performed under conditions such that the remaining peptides each have a new N-terminal amino acid. In some embodiments, the method may further include the step d) removing the next N-terminal amino acid performed under conditions such that the remaining peptides each have a new N-terminal amino acid. In some embodiments, the method may further include the step e) detecting the next signal for each peptide at the single molecule level. In some embodiments, the N-terminal amino acid removing step and the detecting step may be successively repeated from 1 to 20 times. In certain embodiments, the repetitive detection of signal for each peptide at the single molecule level may result in a pattern. In some embodiments, the pattern may be unique to a single peptide within the plurality of immobilized peptides. In some embodiments, the single-peptide pattern may be compared to the proteome of an organism to identify the peptide. In some embodiments, the intensity of the signal may be measured amongst the plurality of immobilized peptides. In some embodiments, the N-terminal amino acids may be removed in step b) by an Edman degradation reaction. In some embodiments, the peptides may be immobilized via cysteine residues. In some embodiments, the detecting in step c) may be done with optics capable of single-molecule resolution. In some embodiments, the degradation step in which removal of the N-terminal amino acid coincides with removal of the polymer may be identified. In some embodiments, the removal of the amino acid measured in step b) may be measured as a reduced fluorescence intensity. In certain embodiments, the methods provided herein may include a) providing a plurality of peptides immobilized on a solid support, each peptide including an N-terminal amino acid and internal amino acids, the internal amino acids comprising lysine, each lysine labeled with a first polymer of the present disclosure, the first polymer producing a first signal for each peptide, and the N-terminal amino acid of each peptide labeled with a second polymer of the present disclosure, the second polymer including a different fluorophore from the first polymer; b) treating the plurality of immobilized peptides under conditions such that each N-terminal amino acid of each peptide is removed; and c) detecting the first signal for each peptide at the single molecule level.

In certain embodiments, the methods provided herein may include a) providing a plurality of peptides immobilized on a solid support, each peptide including an N-terminal amino acid and internal amino acids, the internal amino acids including lysine, each lysine labeled with a first polymer of the present disclosure, the first polymer producing a first signal for each peptide, and the N-terminal amino acid of each peptide labeled with a second polymer of the present disclosure, the second polymer including a different fluorophore from the first polymer, wherein a subset of the plurality of peptides includes an N-terminal acid that is not lysine; b) treating the plurality of immobilized peptides under conditions such that each N-terminal amino acid of each peptide is removed; and c) detecting the first signal for each peptide at the single molecule level under conditions such that the subset of the plurality of peptides includes an N-terminal amino acid that is not lysine is identified.

In certain embodiments, the methods provided herein may include a) digesting a protein preparation with an agent that cleaves after a specific amino acid residue so as to generate a plurality of peptides, each peptide including an N-terminal amino acid and internal amino acids, at least a portion of the internal amino acids of the peptides including lysine, at least a portion of the peptides comprising the specific amino acid residue at a C-terminus; b) labeling the plurality of peptides such that each lysine is labeled with a polymer of the present disclosure, the polymer producing a signal for each peptide; c) immobilizing the labeled peptides on a solid support; d) treating the plurality of immobilized peptides under conditions such that each N-terminal amino acid of each peptide is removed; and e) detecting the signal for each peptide at the single molecule level.

In certain embodiments, the methods provided herein may include a) providing a plurality of immobilized peptides on a solid support, wherein amino acids of an amino acid type of the plurality of immobilized peptides include a polymer of the present disclosure, wherein the amino acid type is at least one of lysine, cysteine, histidine, and tyrosine; b) contacting N-terminal amino acids of the plurality of immobilized peptides with an Edman degradation agent under conditions sufficient to remove the N-terminal amino acids of the plurality of immobilized peptides; c) detecting the fluorophore conjugated to the polymer on amino acids of the amino acid type of the plurality of immobilized peptides; and d) repeating b) and c) one or more times to sequence the plurality of immobilized peptides.

In some embodiments, the detecting may include measuring a fluorescence intensity of the fluorophore. In some embodiments, the plurality of immobilized peptides may be immobilized to the solid support via internal cysteine residues. In some embodiments, the detecting may include measuring an intensity of light emitted from the fluorophore. In some embodiments, d) may include repeating b) and c) at least two times. In some embodiments, an N-terminal amino acid of an immobilized peptide of the plurality of immobilized peptides may be of the amino acid type, wherein the immobilized peptide includes at least one amino acid of the amino acid type separate from the N-terminal amino acid, and wherein in b) the N-terminal amino acid is removed. In some embodiments, a pattern of degradation that coincides with a reduction of signal emitted by the fluorophore may be unique to at least one peptide of the plurality of immobilized peptides. In some embodiments, the pattern may be compared to a proteome of an organism to identify the at least one peptide. In some embodiments, the method may further include, prior to b), contacting the plurality of immobilized peptides with an additional polymer of the present disclosure under conditions sufficient to attach an additional polymer on amino acids of another amino acid type in the plurality of immobilized peptides. In some embodiments, all amino acids of the amino acid type in the plurality of immobilized peptides may include the polymer. In some embodiments, the method may further include, prior to a), (i) providing a sample comprising a plurality of peptides, (ii) contacting the plurality of peptides with a polymer of the present disclosure under conditions sufficient to attach the polymer to the amino acids of the amino acid type, and (iii) immobilizing the plurality of peptides on the solid support, thereby providing the plurality of immobilized peptides. In some embodiments, the amino acid type may be cysteine. In some embodiments, the amino acid type may be histidine. In some embodiments, the amino acid type may be tyrosine. In some embodiments, the Edman degradation agent may be an isothiocyanate derivative selected from the group consisting of phenyl isothiocyanate, fluorescein isothiocyanate, cyanine isothiocyanate, and rhodamine isothiocyanate. In some embodiments, in c) an absence or a reduction in signal intensity may indicate that the polymer has been removed.

In certain embodiments, the methods provided herein may include a) providing a peptide immobilized on a solid support, wherein the peptide includes at least two different types of amino acids coupled to at least two different types of the polymer of the present disclosure; b) subjecting the peptide to conditions sufficient to remove a terminal amino acid of the peptide; and c) detecting the at least two different types of polymer on the at least two different types of amino acids to sequence the peptide. In some embodiments, the at least two different types of amino acids may include lysine. In some embodiments, the at least two different types of amino acids may include a carboxylic acid side chain. In some embodiments, the at least two different types of amino acids may include aspartic acid. In some embodiments, the at least two different types of amino acids may include glutamic acid. In some embodiments, the peptide may be immobilized on the solid support via cysteine residues. In some embodiments, the terminal amino acid may be a N-terminal amino acid. In some embodiments, the terminal amino acid may be a C-terminal amino acid. In certain embodiments, the terminal amino acid of the peptide may be removed by an enzyme. In certain embodiments, the enzyme may include an Edman degradation agent. In some embodiments, the Edman degradation agent may be an isothiocyanate derivative selected from the group consisting of phenyl isothiocyanate, fluorescein isothiocyanate, cyanine isothiocyanate, and rhodamine isothiocyanate.

In some embodiments, the detecting may include measuring a fluorescence intensity of each of the fluorophores conjugated to the at least two different types of polymer. In some embodiments, at least a portion of an emission spectra of each of the fluorophores conjugated to at least two different types of polymer may not overlap with one another. In certain embodiments, in c), a reduction in signal intensity may indicate that at least one amino acid of the at least two different types of amino acids coupled to the at least two different types of the polymer has been removed. In some embodiments, in c), an absence in signal intensity may indicate that the at least two different types of amino acids coupled to the at least two different types of the polymer have been removed.

In certain embodiments, dye quenching between a first fluorophore conjugated to a first polymer of the at least two different types of polymer and a second fluorophore conjugated to a second polymer of the at least two different types of polymer may be reduced compared to dye quenching between identical fluorophores conjugated directly to the at least two different types of amino acids. In some embodiments, the first fluorophore may be Atto647N and the second fluorophore may be Atto647N.

In some embodiments, Förster resonance energy transfer (FRET) between a first fluorophore conjugated to a first polymer of the at least two different types of polymer and a second fluorophore conjugated to a second polymer of the at least two different types of polymer may be reduced compared to FRET between identical fluorophores conjugated directly to the at least two different types of amino acids. In some embodiments, the first fluorophore may be Atto647N and the second fluorophore may be Janelia Fluor® 549.

In certain embodiments, the method may further include, prior to b), contacting the peptide immobilized on the solid support with an additional polymer of the present disclosure under conditions sufficient to couple the additional polymer to another type of amino acid different from the at least two different types of amino acids. In some embodiments, the peptide may include at least three different types of amino acids coupled to at least three different types of polymer.

In certain embodiments, the methods provided herein may include a) providing the polypeptide; b) contacting the polypeptide with a first polymer configured to couple with a first amino acid of the polypeptide; c) contacting the polypeptide with a second polymer configured to couple with a second amino acid of the polypeptide; d) immobilizing the polypeptide directly or indirectly to a support; e) subjecting the polypeptide to conditions sufficient to remove at least one amino acid from the polypeptide; f) detecting a signal or a signal change associated with the first polymer or the second polymer from the polypeptide; and g) identifying, using at least one of the signal or the signal change, at least a portion of the sequence of the polypeptide; wherein the first amino acid has greater nucleophilicity than the second amino acid; wherein step b) occurs before step c); and wherein the first and the second polymer are the polymer of the present disclosure.

In some embodiments, a) the first amino acid may include cysteine and the second amino acid may include lysine; or b) the first amino acid may include cysteine and the second amino acid may include glutamic acid and aspartic acid; or c) the first amino acid may include tyrosine and the second amino acid may include glutamic acid and aspartic acid. In some embodiments, the at least one amino acid may be removed from an N-terminus of the polypeptide. In some embodiments, the first amino acid or the second amino acid may include a plurality of amino acids, and wherein the at least one signal or signal change may include a collective signal from the polypeptide and associated with a plurality of first polymers or a plurality of second polymers coupled thereto. In certain embodiments, the first polymer and the second polymer may generate different signals or signal changes. In some embodiments, the signal or the signal change may include a plurality of signals of different intensities. In some embodiments, the signal or the signal change may be detected with an optical detector having single-molecule sensitivity. In some embodiments, the first polymer may be configured to covalently couple to the first amino acid and the second polymer may be configured to covalently couple to the second amino acid. In some embodiments, step b) may occur before step d).

In certain embodiments, dye quenching between a fluorophore conjugated to the first polymer and a fluorophore conjugated to the second polymer may be reduced compared to dye quenching between identical fluorophores conjugated directly to the first amino acid and the second amino acid. In some embodiments, the fluorophore conjugated to the first polymer may be Atto647N and the fluorophore conjugated to the second polymer may be Atto647N. In some embodiments, Förster resonance energy transfer (FRET) between a fluorophore conjugated to the first polymer and a fluorophore conjugated to the second polymer may be reduced compared to FRET between identical fluorophores conjugated directly to the first amino acid and the second amino acid. In some embodiments, the fluorophore conjugated to the first polymer may be Atto647N and the fluorophore conjugated to the second polymer may be Janelia Fluor® 549.

### b) Synthesis of Polymers

In certain embodiments, the methods provided herein may include (a) synthesizing a peptide of a sequence Fmoc-flexible spacer-rigid polypeptide-amino acid residue (boc)-CONH₂ using a solid phase peptide synthesizer; (b) removing the Fmoc group and conjugating a functional group to a first end of the polymer; and (c) conjugating a fluorophore to a second end of the polymer via the amino acid residue. In some embodiments, the amino acid residue may be a lysine residue. In some embodiments, the functional group may be a click-reactive group. In some embodiments, the sequence Fmoc-flexible spacer-rigid polypeptide-amino acid residue (boc)-CONH₂ may be Fmoc-Gly-(O—CH₂—CH₂)₂-Gly-Pro₃₀-Lys(boc)-CONH₂. In some embodiments, the functional group may be DBCO, wherein the DBCO may be conjugated via a DBCO-NHS molecule. In some embodiments, the fluorophore may be Atto643, wherein the Atto643 may be conjugated via a Atto643-NHS ester molecule.

c) Labeling with Polymers

In certain embodiments, the methods provided herein may include (a) providing a peptide, wherein the peptide comprises an internal amino acid coupled to an azide and a C-terminus coupled to an alkyne; and (b) bringing the peptide in contact with a first polymer of the present disclosure under conditions such that the first polymer reacts with the internal amino acid, wherein the first polymer includes a functional group that is a strained alkyne. In some embodiments, (b) may be performed in the absence of copper (Cu). In some embodiments, the method may further include (c) reacting a second polymer of the present disclosure that is different from the first polymer with the C-terminus, wherein the second polymer includes a functional group that is a non-strained alkyne. In certain embodiments, (c) may be performed in the presence of copper (Cu). In some embodiments, the azide coupled to the internal amino acid may not react with the alkyne coupled to the C-terminus.

In certain embodiments, the methods provided herein may include (a) incubating a polymer of the present disclosure with a peptide including an amino acid under conditions sufficient to react the functional group of the polymer with the peptide; and (b) purifying the peptide.

In some embodiments, the amino acid may include azidolysine. In some embodiments, the peptide may include at least one lysine, and the method may further include: (c) functionalizing the lysine with NHS—(O—CH₂—CH₂)₄-azide; and (d) incubating a second polymer of the present disclosure with the peptide under conditions sufficient to react the functional group of the second polymer with the peptide. In some embodiments, the peptide may be labeled with two or more polymers in a bottle brush configuration.

In some embodiments, a kit for labeling an amino acid of a peptide is provided. In some embodiments, the kit may include: (a) at least one polymer of the present disclosure, wherein the functional group of the polymer is configured to couple to an amino acid of an amino acid type; and (b) instructions for use to couple the polymer to the amino acid.

### V. Peptide Degradation

The present disclosure provides a range of chemical and enzymatic techniques for mild and sequential protein degradation. Degradation can be utilized in a range of peptide sequencing and analysis methods, for example to determine the order or identity of particular amino acids in a fluorosequencing assay. A peptide or protein may be iteratively subjected to cleavage conditions to determine the sequence of at least a portion of its sequence. The entire sequence of a peptide may be determined using the methods and compositions described herein. Controlled amino acid removal (e.g., N- or C-terminal amino acid removal) may be carried out through a variety of techniques including, for example, Edman degradation, organophosphate degradation, or proteolytic cleavage. In some instances, Edman degradation is used to remove a single terminal amino acid residue from a peptide N- or C-terminus. In some instances, the N-terminal amino acid residue is selectively removed from a peptide. A chemical or enzymatic technique for removing a terminal amino acid may remove a defined number of (e.g., exactly one, exactly two, at most two) amino acids. Accordingly, a method for analyzing a peptide may include successive degradation and analysis steps, such that the removal of a defined number of amino acids from an N-terminus or C-terminus per step provides position and sequence specific amino acid identifications during analysis. A chemical or enzymatic technique for removing a terminal amino acid may cleave a peptide at a defined location (e.g., only in between two alanine residues, or only at the peptide bond connecting an N-terminal amino acid to the remainder of a peptide).

An Edman degradation method may include chemically functionalizing a peptide N-terminus or C-terminus (e.g., to form a thiourea or a guanidinium derivative of an N-terminal amine), and then contacting the functionalized terminal amino acid with a reagent (e.g., a hydrazine), a condition (e.g., a high or low pH or temperature), or an enzyme (e.g., an Edmanase with specificity for the functionalized terminal amino acid) to remove the functionalized terminal amino acid.

A diactivated phosphate or phosphonate may be used for peptide cleavage. Such a method may utilize an acid to remove a functionalized amino acid. The diactivated phosphate or phosphonate may be a dihalophosphate ester. In other embodiments, the techniques involve using an enzyme to remove the terminal amino acid residue, such as, for example, an exopeptidase or an Edmanase. For example, a method may include derivatizing an N-terminal amino acid of a peptide with a diactivated phosphate, and contacting the peptide with an Edmanase with cleavage activity toward phosphate-functionalized N-terminal amino acids.

A cleavage method (e.g., a cleavage method implemented within a sequencing method) may comprise enzymatic cleavage. The cleavage method may comprise the use of a single protease, a series of proteases (e.g., provided in a specific order), or a combination of proteases. Exemplary proteases and their associated cleavage sites are provided in Table 1.

TABLE 1
Exemplary Proteases

| Protease | Cleavage Site |
|---|---|
| Carboxypeptidase A | C-terminal exopeptidase |
| Carboxypeptidase B | C-terminal exopeptidase; specific for lysine or arginine |
| Carboxypeptidase P | C-terminal exopeptidase |
| Carboxypeptidase Y | C-terminal exopeptidase |
| Cathepsin C | N-terminal exopeptidase; removes N-terminal dipeptide (except when |
| | N-terminal amino acid is lysine or arginine, or when 2^{nd} or 3^{rd} amino |
| | acid from N-terminal is proline) |
| Chymotrypsin | C-terminal exopeptidase; specific for phenylalanine, tryptophan, and |
| | tyrosine |
| Clostripain | Arginine |
| Elastase | Alanine, Valine, Serine, Glycine, Leucine, or Isoleucine |
| Endoproteinase Arg-C | Arginine |
| Endoproteinase Glu-C | Glutamic Acid |
| Endoproteinase Lys-C | Lysine |
| Glutamylendopeptidase | Glutamic acid |
| Kallikrein (Plasma) | Lysine or Arginine |
| Papain | Lysine or Arginine followed by a hydrophobic residue |
| Pepsin | Leucine, Phenylalanine, Tryptophan or Tyrosine |
| Proteinase K | Aliphatic and aromatic amino acids |
| Subtilisin | Hydrophobic amino acids |
| TEV Protease | Specific for the sequences Glutamic acid-Asparagine-Leucine- |
| | Tyrosine-Phenylalanine-Glutamine-Glycine and Glutamic acid- |
| | Asparagine- Leucine-Tyrosine- Phenylalanine-Glutamine-Serine; |
| | cleaves between Glutamine-Glycine or Glutamine-Serine |
| Thermolysin | Isoleucine, Methionine, Phenylalanine, Tryptophan, Tyrosine, or |
| | Valine |
| Trypsin | Lysine or Arginine |

Peptide cleavage may include chemical cleavage. Examples of chemical cleavage reagents consistent with the present disclosure include cyanogen bromide, BNPS-skatole, formic acid, hydroxylamine, and 2-nitro-5-thiocyanobenzoic acid. A cleavage method may include a combination (e.g., parallel or sequential use) of chemical and enzymatic cleavage reagents. A cleavage method may include activating (e.g., functionalizing) an amino acid for chemical or enzymatic cleavage. For example, a method may include derivatizing an N-terminal amino acid residue of a peptide, and then contacting the peptide with an ‘Edmanase’ enzyme configured to remove the derivatized N-terminal amino acid residue.

Peptide cleavage conditions may be achieved with a solvent. The solvent may be an aqueous solvent, an organic solvent, or a combination or mixture thereof. The solvent may be an organic solvent. The organic solvent may include a miscibility with water. The organic solvent may be anhydrous. The solvent may be a non-polar solvent (e.g., hexane, dichloromethane (DCM), diethyl ether, etc.), a polar aprotic solvent (e.g., tetrahydrofuran (THF), ethyl acetate, dimethylformamide (DMF), acetonitrile (MeCN), dimethyl sulfoxide (DMSO), etc.), or a polar protic solvent (e.g., isopropanol (IPA), ethanol, methanol, acetic acid, water, etc.). The solvent may be DMF. The solvent may be a C₁-C₁₂ haloalkane. The C₁-C₁₂ haloalkane may be DCM. The solvent may be a mixture of two or more solvents. The mixture of two or more solvents may be a mixture of a polar aprotic solvent and a C₁-C₁₂ haloalkane. The mixture of two or more solvents may be a mixture of DMF and DCM. The mixture of solvents may be any combination thereof.

A degradation process may include a plurality of steps. For example, a method may include an initial step for derivatizing a terminal amino acid of a peptide, and a subsequent step for cleaving the derivatized terminal amino acid from the peptide. One such method includes organophosphorus compound-mediated N-terminal functionalization and removal, and thus provides an alternative to the isothiocyanate (e.g., phenyl isothiocyanate) based processes of some Edman degradation schemes.

An organophosphate-based degradation scheme may include dissolving a peptide in an organic solvent or organic solvent mixture (e.g., a mixture of dichloromethane and dimethylformamide) in the presence of an organic base (e.g., triethylamine, N, N-diisopropylethylamine (DIPEA), 1,8-diazabicyclo[5.4.0]undec-7-ene (DBU), pyridine, 1,5-diazabicyclo(4.3.0)non-5-ene, 2,6-di-tert-butylpyridine, imidazole, histidine, sodium carbonate, and the like). The peptide may then be contacted with at least one organophosphorus compound. The cleavage of the peptide or protein N-terminus may be initiated through the addition of a weak acid (e.g., formic acid in water). The cleavage of the peptide or protein N-terminus may also be initiated with water. The resulting products may include the terminal amino acid of the peptide or protein released from the peptide as a phosphoramide and the peptide or protein that is shortened by the terminal amino acid residue, which comprises a free N-terminus that can be used to perform a subsequent cleavage reaction.

A cleavage method may include digesting a peptide to generate fragments of a desired average length. The cleavage method may generate peptides (e.g., by acting upon a complex mixture of peptides, such as cell lysate) with an average length of at least 5 amino acids, at least 8 amino acids, at least 10 amino acids, at least 12 amino acids, at least 15 amino acids, at least 20 amino acids, at least 25 amino acids, at least 30 amino acids, at least 40 amino acids, or at least 50 amino acids. The cleavage method may generate peptides with an average length of at most 50 amino acids, at most 40 amino acids, at most 30 amino acids, at most 25 amino acids, at most 20 amino acids, at most 15 amino acids, at most 12 amino acids, at most 10 amino acids, at most 8 amino acids, or at most 5 amino acids. The cleavage method may generate peptide fragments with an average length of between 5 and 20 amino acids, between 5 and 30 amino acids, between 10 and 20 amino acids, between 10 and 30 amino acids, between 12 and 18 amino acids, between 15 and 30 amino acids, between 20 and 40 amino acids, or between 30 and 50 amino acids.

A reaction mixture may include a stoichiometric or an excess concentration of a cleavage compound (e.g., relative to the concentration of peptides to be cleaved). The reaction mixture may include at least about 0.001% v/v, about 0.01% v/v, about 0.1% v/v, about 1% v/v, about 5% v/v, about 10% v/v, about 15% v/v, about 20% v/v, about 30% v/v, about 40% v/v, about 50% v/v, or more of the cleavage compound. The reaction mixture may include at most about 50% v/v, about 40% v/v, about 30% v/v, about 20% v/v, about 15% v/v, about 10% v/v, about 5% v/v, about 1% v/v, about 0.1% v/v, about 0.01% v/v, about 0.001% v/v, or less of the cleavage compound. The reaction mixture may include from about 0.1% v/v to about 20% v/v, about 0.5% v/v to about 10% v/v, or about 1% v/v to about 10% v/v of the cleavage compound. The reaction mixture may include about 5% v/v of the cleavage compound.

The reaction may be performed at a temperature of at least about 0° C., at least about 5° C., at least about 10° C., at least about 15° C., at least about 20° C., at least about 25° C., at least about 30° C., at least about 40° C., at least about 50° C., at least about 60° C., at least about 70° C., at least about 80° C., or at least about 90° C. The reaction may be performed at a temperature of at most about 90° C., at most about 80° C., at most about 70° C., about 60° C., about 50° C., about 40° C., about 30° C., about 25° C., about 20° C., about 15° C., about 10° C., about 5° C., about 0° C., or less. The reaction may be performed at a temperature from about 0° C. to about 70° C., about 10° C. to about 50° C., about 20° C. to about 40° C., or about 20° C. to about 30° C. The reaction may be performed at a temperature above room temperature (e.g., about 22° C. to about 27° C.). The reaction may be performed at room temperature. The reaction may be performed at close to 0° C. or below 0° C. (e.g., in the presence of an antifreeze).

The peptide and the cleavage compound may be mixed or incubated for at least about 1 minute, about 5 minutes, about 10 minutes, about 20 minutes, about 30 minutes, about 40 minutes, about 50 minutes, about 60 minutes, about 2 hours, about 3 hours, about 4 hours, about 6 hours, about 8 hours, about 10 hours, about 12 hours, about 16 hours, about 20 hours, about 24 hours, or more. The peptide and the cleavage compound may be mixed or incubated for at most about 24 hours, about 20 hours, about 16 hours, about 12 hours, about 10 hours, about 8 hours, about 6 hours, about 4 hours, about 3 hours, about 2 hours, about 1 hour, about 50 minutes, about 40 minutes, about 30 minutes, about 20 minutes, about 10 minutes, about 5 minutes, about 1 minute, or less. The peptide and the cleavage compound may be mixed or incubated from about 1 minute to about 24 hours, 5 minutes to about 6 hours, 5 minutes to about 2 hours, or 5 minutes to about 30 minutes.

In some embodiments, an N-terminal amino acid residue may be selectively removed from a peptide, wherein the N-terminal amino acid includes an amino-acid side chain that is attached to a polymer of the present disclosure. In some embodiments, the amino-acid side chain is attached to the polymer via the functional group.

### VI. Enumerated Embodiments

The present disclosure provides the following non-limiting enumerated Embodiments.

Embodiment 1. A polymer, comprising: a functional group; a backbone; and an amino acid residue conjugated to a fluorophore.

Embodiment 2. A polymer, comprising: a functional group configured to couple to a polypeptide or protein; a backbone; and an amino acid residue conjugated to a fluorophore.

Embodiment 3. The polymer of Embodiment 1 or Embodiment 2, wherein the backbone comprises a repeating monomer subunit.

Embodiment 4. The polymer of Embodiment 3, wherein the monomer subunit is an amino acid residue, ethylene glycol, a proline residue, a proline residue derivative, a natural amino acid, an unnatural amino acid, a polysarcosine, a polyalanine a copolymer, or any combination thereof.

Embodiment 5. The polymer of any one of Embodiments 1-4, wherein the backbone comprises a rigid polypeptide comprising at least ten amino acid residues.

Embodiment 6. The polymer of any one of Embodiments 1-5, wherein the backbone further comprises a flexible spacer.

Embodiment 7. The polymer of Embodiment 6, wherein the polymer comprises the functional group, the flexible spacer, the rigid polypeptide, and the amino acid residue conjugated to a fluorophore in amino-terminal to carboxy-terminal direction or carboxy-terminal to amino-terminal direction.

Embodiment 8. The polymer of any one of Embodiments 5-7 wherein at least one amino acid residue of the at least ten amino acid residues of the rigid polypeptide is a natural amino acid residue.

Embodiment 9. The polymer of any one of Embodiments 5-8, wherein at least one amino acid residue of the at least ten amino acid residues of the rigid polypeptide is an unnatural amino acid residue.

Embodiment 10. The polymer of Embodiment 9, wherein the unnatural amino acid residue is a proline residue derivative.

Embodiment 11. The polymer of Embodiment 10, wherein the proline residue derivative is hydroxyproline.

Embodiment 12. The polymer of any one of Embodiments 1-11, wherein the polymer comprises an amino acid residue that is cationic, anionic, zwitterionic, or any combination thereof.

Embodiment 13. The polymer of any one of Embodiment 1-12, wherein the polymer comprises an arginine, an alanine, glutamic acid, or any combination thereof.

Embodiment 14. The polymer of any one of Embodiments 1-13, wherein the polymer comprises a phenylsulfonic acid, a polyglutamic acid, a polysarcosine, a polyalanine, or any combination thereof.

Embodiment 15. The polymer of any one of Embodiments 1-14, further comprising an antioxidant group.

Embodiment 16. The polymer of Embodiment 15, wherein the antioxidant group comprises p-nitrophenylalanine, trolox, cyclooctatetraene, or any combination thereof, and optionally, is p-nitrophenylalanine, trolox, cyclooctatetraene, or any combination thereof.

Embodiment 17. The polymer of any one of Embodiments 1-16, further comprising a metal chelator.

Embodiment 18. The polymer of any one of Embodiments 1-14, wherein the flexible spacer comprises (O—CH₂—CH₂)ₙ.

Embodiment 19. The polymer of any one of Embodiments 1-18, wherein the flexible spacer comprises Gly-(O—CH₂—CH₂)ₙ-Gly.

Embodiment 20. The polymer of Embodiment 18 or Embodiment 19, wherein n is a value between 1-23, inclusive.

Embodiment 21. The polymer of Embodiment 18 or Embodiment 19, wherein n is 2.

Embodiment 22. The polymer of any one of Embodiments 1-21, wherein the flexible spacer comprises an alkyl chain.

Embodiment 23. The polymer of Embodiment 22, wherein the alkyl chain comprises 6-aminohexanoic acid, 12-aminododecanoic acid, or a combination thereof.

Embodiment 24. The polymer of any one of Embodiments 1-23, wherein the flexible spacer comprises sarcosine (N-methylglycine) copolymerized with alanine, serine, or a combination thereof.

Embodiment 25. The polymer of any one of Embodiments 1-24, wherein the rigid polypeptide comprises at least ten proline residues, proline residue derivatives, or a combination thereof.

Embodiment 26. The polymer of any one of Embodiments 1-25, wherein the rigid polypeptide comprises from about ten to about 40 proline residues, proline residue derivatives, or a combination thereof.

Embodiment 27. The polymer of any one of Embodiments 1-26, wherein the rigid polypeptide comprises at least 14 proline residues, proline residue derivatives, or a combination thereof.

Embodiment 28. The polymer of any one of Embodiments 1-27, wherein the rigid polypeptide comprises at least 25 proline residues, proline residue derivatives, or a combination thereof.

Embodiment 29. The polymer of any one of Embodiments 1-25, wherein the rigid polypeptide comprises at least 30 proline residues, proline residue derivatives, or a combination thereof.

Embodiment 30. The polymer of any one of Embodiments 1-29, wherein the rigid polypeptide comprises or consists of 30 proline residues, proline residue derivatives, or a combination thereof.

Embodiment 31. The polymer of any one of Embodiments 1-30, wherein the rigid polypeptide comprises a polyproline of between about ten and about 40 consecutive proline residues.

Embodiment 32. The polymer of any one of Embodiments 1-31, wherein the rigid polypeptide comprises a polyproline of at least ten, at least 14, at least 25, at least 30, or 30 consecutive proline residues.

Embodiment 33. The polymer of any one of Embodiments 1-32, wherein the rigid polypeptide has a length from about 2 nm to about 12 nm.

Embodiment 34. The polymer of Embodiment 33, wherein the rigid polypeptide has a length from about 6 nm to about 9 nm.

Embodiment 35. The polymer of any one of Embodiments 1-34, wherein the amino acid residue conjugated to the fluorophore comprises a lysine residue, a cysteine residue, or an azidolysine residue, and optionally, is a lysine residue, a cysteine residue, or an azidolysine residue.

Embodiment 36. The polymer of any one of Embodiments 1-35, wherein the amino acid residue conjugated to the fluorophore is a lysine residue.

Embodiment 37. The polymer of any one of Embodiments 1-36, wherein the fluorophore comprises an Alexa Fluor® dye, an Atto dye, a Janelia Fluor® dye, a carbopyronine derivative, a cyanine derivative, a Rhodamine derivative, or any combination thereof, and optionally, is an Alexa Fluor® dye, an Atto dye, a Janelia Fluor® dye, a carbopyronine derivative, a cyanine derivative, a Rhodamine derivative, or any combination thereof.

Embodiment 38. The polymer of any one of Embodiments 1-37, wherein the fluorophore is Alexa Fluor® 405, Alexa Fluor® 448, Alexa Fluor® 555, Alexa Fluor® 594, Alexa Fluor® 647, Alexa Fluor® 680, Atto390, Atto425, Atto488, Atto495, Atto514, Atto550, Atto647N, Atto643, Atto532, Atto647, Atto655, Atto680, Atto700, (5)6-napthofluorescein, Oregon Green™ 488, Oregon Green™ 514, JFX554, 00488-NHS, 00488-Azide, 00488-Tetrazine, 00514-NHS, Janelia Fluor® 479, Janelia Fluor® 525, Janelia Fluor® 549, Janelia Fluor® 555, Janelia Fluor® 579, SF554, Texas Red, JFX 554, JFX 650, CFR 398, CF® 430, CFR 568, CFR 633, CFR 640R, CFR 680R, Sarafluor™ 488B, Sarafluor™ 650B, Rhodamine, Rhodamine 110 (5-CR110), Rhodamine 6G, Rhodamine B, carboxyrhodamine B, tetramethylrhodamine (TMR), Rhodamine 101, Rhodamine Si, fluorescein, 5-carboxyfluorescein, napthofluorescein, 6-JOE-N3, 7-hydroxycoumarin-N3, Cy3 Cy5, Cy3B, Cy5B, Cy488, Dylight™ 405, Dylight™ 488, iFluor® 710, DY350XL, DY351XL, DY360XL, DY370XL, DY376XL, DY380XL, DY396XL, DY720, Bodipy™ 493, Bodipy™ FL, Bodipy IM 650, PB430 (Phoxbright 430), Pyrene-N3, Lucifer Yellow, Nanohoop 6, Nanohoop 8, Eterneon 394, Hilyte™ 405, Hilyte™ 488, Hilyte™ 647, or any combination thereof.

Embodiment 39. The polymer of any one of Embodiments 1-38, wherein the fluorophore is Texas Red, Janelia Fluor® 525, Janelia Fluor® 549, Janelia Fluor® 555, Alexa Fluor® 448, Alexa Fluor® 555, Atto495, Atto643, Atto647N, Rhodamine, tetramethylrhodamine, or any combination thereof. 30

Embodiment 40. The polymer of any one of Embodiments 1-39, wherein the fluorophore is Texas Red, Janelia Fluor® 549, Alexa Fluor® 555, Atto643, or any combination thereof.

Embodiment 41. The polymer of any one of Embodiments 1-40, further comprising at least one additional amino acid residue, wherein the additional amino acid residue is conjugated to an additional fluorophore.

Embodiment 42. The polymer of Embodiment 41, wherein the additional amino acid residue conjugated to the additional fluorophore is positioned adjacent to the amino acid residue conjugated to the fluorophore.

Embodiment 43. The polymer of Embodiment 41 or 42, wherein the fluorophores are the same fluorophore.

Embodiment 44. The polymer of Embodiment 41 or 42, wherein the fluorophores are different fluorophores.

Embodiment 45. The polymer of any one of Embodiments 1-44, wherein the functional group is a iodoacetamide, a maleimide, an amine, an azide, an alkene, an aldehyde, a ketone, a tetrazine, an alkyne, a strained alkyne, a cycloalkyne, a cyclooctyne, dibenzocyclooctyne (DBCO), a thiol, a carboxyl, a hydrazide, a dithiol, a trans-cyclooctene, a bicycloalkene, an iodobenzene, a cyanothiazole, an acene, a dithiolane, a bromane, an aminothiol, a pyrroledione, a sulfenyl chloride, a succinimidyl ester, a succinidimyl ester, methyltetrazine, lipoic acid, or any combination thereof.

Embodiment 46. The polymer of Embodiment 45, wherein the functional group is a strained alkyne.

Embodiment 47. The polymer of Embodiment 45, wherein the functional group is dibenzocyclooctyne (DBCO), methyltetrazine or lipoic acid.

Embodiment 48. The polymer of Embodiment 45, wherein the functional group is dibenzocyclooctyne (DBCO).

Embodiment 49. The polymer of any one of Embodiments 1-48, wherein the functional group comprises a clack group.

Embodiment 50. The polymer of Embodiment 49, wherein the clack group is configured to react with a click group on a peptide.

Embodiment 51. A polymer, comprising in amino-terminal to carboxy-terminal direction:
a functional group that is dibenzocyclooctyne (DBCO);
a flexible spacer comprising Gly-(O—CH₂—CH₂)₂-Gly;
a rigid polypeptide comprising 30 proline residues; and
a lysine residue conjugated to a fluorophore.

Embodiment 52. A polymer comprising or consisting of structure I:

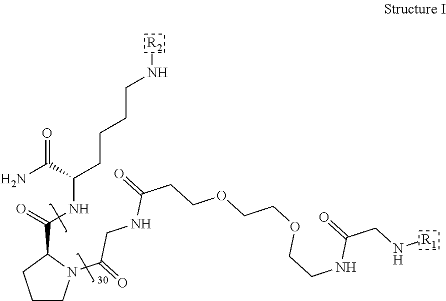

wherein R₁=DBCO and R₂=a fluorophore.

Embodiment 53. The polymer of any one of Embodiments 1-7, wherein the polymer has a sequence of DBCO-Gly-(O—CH₂—CH₂)₂-Gly-Pro₃₀-Lys(fluorophore)-CONH₂.

Embodiment 54. The polymer of any one of Embodiments 1-53, wherein the polymer is synthesized using a solid-phase peptide synthesizer.

Embodiment 55. A peptide-polymer conjugate comprising:
the polymer of any one of Embodiments 1-54;
a peptide with at least one amino-acid side chain, wherein the amino-acid side chain is attached to the polymer via the functional group.

Embodiment 56. The peptide-polymer conjugate of Embodiment 55, wherein at least two polymers are attached to the peptide via two different amino-acid side chains.

Embodiment 57. The peptide-polymer conjugate of Embodiment 56, wherein the at least two polymers are attached to the peptide in a bottle brush configuration.

Embodiment 58. The peptide-polymer conjugate of Embodiment 56 or Embodiment 57, wherein the at least two polymers comprise the same fluorophore, and dye quenching between the fluorophores is reduced compared to dye quenching between identical fluorophores attached directly to the amino-acid side chains of the peptide.

Embodiment 59. The peptide-polymer conjugate of Embodiment 56 or Embodiment 57, wherein the at least two polymers comprise different fluorophores, and wherein Förster resonance energy transfer (FRET) between the fluorophores is reduced compared to FRET between identical fluorophores attached directly to the amino-acid side chains of the peptide.

Embodiment 60. A composition comprising at least one polymer of any one of Embodiments 1-54 and a solvent.

Embodiment 61. A method of reducing dye-dye interactions, comprising:
a) providing a polymer of any one of Embodiments 1-54;
b) providing a biomolecule comprising at least two reactive groups; and
c) attaching at least two polymers to the at least two reactive groups of the biomolecule via the functional group, wherein the two polymers comprise the same fluorophore;
thereby reducing dye-dye interactions between identical fluorophores compared to dye-dye interactions between identical fluorophores conjugated directly to the at least two reactive groups of the biomolecule.

Embodiment 62. The method of Embodiment 61, wherein the biomolecule is a peptide.

Embodiment 63. The method of Embodiment 61 or 62, wherein the reactive group is an amino-acid side chain.

Embodiment 64. The method of anyone of Embodiments 61-63, wherein dye-dye interactions are reduced by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to dye-dye interactions between identical fluorophores conjugated directly to the at least two reactive groups of the biomolecule.

Embodiment 65. A method of reducing dye quenching, comprising:
a) providing a polymer of any one of Embodiments 1-54;
b) providing a peptide with at least two amino-acid side chains; and
c) attaching at least two polymers to the at least two amino-acid side chains via the functional group, wherein the two polymers comprise the same fluorophore;
thereby reducing dye quenching between identical fluorophores compared to dye quenching between identical fluorophores conjugated directly to the at least two amino-acid side chains of the peptide.

Embodiment 66. The method of Embodiment 65, wherein the identical fluorophores are Atto647N.

Embodiment 67. The method of Embodiment 65 or Embodiment 66, wherein dye quenching is reduced by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to dye quenching between identical fluorophores conjugated directly to the at least two amino-acid side chains of the peptide.

Embodiment 68. The method of Embodiment 67, wherein dye quenching is reduced by at least about 70%.

Embodiment 69. A method of reducing Förster resonance energy transfer (FRET), comprising:
a) providing a polymer of any one of Embodiments 1-54;
b) providing a peptide with at least two amino-acid side chains; and
c) attaching at least two polymers to the at least two amino-acid side chains via the functional group, wherein the two polymers comprise different fluorophores;
thereby reducing FRET between the fluorophores compared to FRET between equivalent fluorophores conjugated directly to the at least two amino-acid side chains of the peptide.

Embodiment 70. The method of Embodiment 69, wherein the different fluorophores are Atto647N and Janelia Fluor® 549.

Embodiment 71. The method of Embodiment 69 or Embodiment 70, wherein FRET is reduced by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to FRET between equivalent fluorophores conjugated directly to the at least two amino-acid side chains of the peptide.

Embodiment 72. The method of Embodiment 71, wherein FRET is reduced by at least about 90%.

Embodiment 73. A method of treating peptides, comprising:
a) providing a plurality of peptides immobilized on a solid support, each peptide comprising an N-terminal amino acid and internal amino acids, the internal amino acids comprising lysine, each lysine labeled with the polymer of any one of Embodiments 1-54, and the polymer producing a signal for each peptide;
b) treating the plurality of immobilized peptides under conditions such that each N-terminal amino acid of each peptide is removed; and
c) detecting the signal for each peptide at the single molecule level.

Embodiment 74. The method of Embodiment 73, wherein in step b) the N-terminal amino acid of each peptide is reacted with a phenyl isothiocyanate derivative.

Embodiment 75. The method of Embodiment 73 or Embodiment 74, wherein the removal of the N-terminal amino acid in step b) is performed under conditions such that the remaining peptides each have a new N-terminal amino acid.

Embodiment 76. The method of Embodiment 75, further comprising the step d) removing the next N-terminal amino acid performed under conditions such that the remaining peptides each have a new N-terminal amino acid.

Embodiment 77. The method of Embodiment 76, further comprising the step e) detecting the next signal for each peptide at the single molecule level.

Embodiment 78. The method of Embodiment 77, wherein the N-terminal amino acid removing step and the detecting step are successively repeated from 1 to 20 times.

Embodiment 79. The method of Embodiment 78, wherein the repetitive detection of signal for each peptide at the single molecule level results in a pattern.

Embodiment 80. The method of Embodiment 79, wherein the pattern is unique to a single-peptide within the plurality of immobilized peptides.

Embodiment 81. The method of Embodiment 80, wherein the single-peptide pattern is compared to the proteome of an organism to identify the peptide.

Embodiment 82. The method of any one of Embodiments 73-81, wherein the intensity of the signal is measured amongst the plurality of immobilized peptides.

Embodiment 83. The method of any one of Embodiments 73-82, wherein the N-terminal amino acids are removed in step b) by an Edman degradation reaction.

Embodiment 84. The method of any one of Embodiments 73-83, wherein the peptides are immobilized via cysteine residues.

Embodiment 85. The method of any one of Embodiments 73-84, wherein the detecting in step c) is done with optics capable of single-molecule resolution.

Embodiment 86. The method of Embodiment 83, wherein the degradation step in which removal of the N-terminal amino acid coincides with removal of the polymer is identified.

Embodiment 87. The method of Embodiment 86, wherein the removal of the amino acid is measured in step b) is measured as a reduced fluorescence intensity.

Embodiment 88. A method of treating peptides, comprising:
a) providing a plurality of peptides immobilized on a solid support, each peptide comprising an N-terminal amino acid and internal amino acids, the internal amino acids comprising lysine, each lysine labeled with a first polymer of any one of Embodiments 1-54, the first polymer producing a first signal for each peptide, and the N-terminal amino acid of each peptide labeled with a second polymer of any one of Embodiments 1-54, the second polymer comprising a different fluorophore from the first polymer;
b) treating the plurality of immobilized peptides under conditions such that each N-terminal amino acid of each peptide is removed; and
c) detecting the first signal for each peptide at the single molecule level.

Embodiment 89. A method of identifying amino acids in peptides, comprising:
a) providing a plurality of peptides immobilized on a solid support, each peptide comprising an N-terminal amino acid and internal amino acids, the internal amino acids comprising lysine, each lysine labeled with a first polymer of any one of Embodiments 1-54, the first polymer producing a first signal for each peptide, and the N-terminal amino acid of each peptide labeled with a second polymer of any one of Embodiments 1-54, the second polymer comprising a different fluorophore from the first polymer, wherein a subset of the plurality of peptides comprises an N-terminal acid that is not lysine;
b) treating the plurality of immobilized peptides under conditions such that each N-terminal amino acid of each peptide is removed; and
c) detecting the first signal for each peptide at the single molecule level under conditions such that the subset of the plurality of peptides comprising an N-terminal amino acid that is not lysine is identified.

Embodiment 90. A method of generating and treating peptides, comprising:
a) digesting a protein preparation with an agent that cleaves after a specific amino acid residue so as to generate a plurality of peptides, each peptide comprises an N-terminal amino acid and internal amino acids, at least a portion of the internal amino acids of the peptides comprising lysine, at least a portion of the peptides comprising the specific amino acid residue at a C-terminus;
b) labeling the plurality of peptides such that each lysine is labeled with a polymer of any one of Embodiments 1-54, the polymer producing a signal for each peptide;
c) immobilizing the labeled peptides on a solid support;
d) treating the plurality of immobilized peptides under conditions such that each N-terminal amino acid of each peptide is removed; and
e) detecting the signal for each peptide at the single molecule level.

Embodiment 91. A method for peptide sequencing, comprising:
a) providing a plurality of immobilized peptides on a solid support, wherein amino acids of an amino acid type of the plurality of immobilized peptides comprise a polymer of any one of Embodiments 1-54, wherein the amino acid type is at least one of lysine, cysteine, histidine, and tyrosine;
b) contacting N-terminal amino acids of the plurality of immobilized peptides with an Edman degradation agent under conditions sufficient to remove the N-terminal amino acids of the plurality of immobilized peptides;
c) detecting the fluorophore conjugated to the polymer on amino acids of the amino acid type of the plurality of immobilized peptides; and
d) repeating b) and c) one or more times to sequence the plurality of immobilized peptides.

Embodiment 92. The method of Embodiment 91, wherein the detecting comprises measuring a fluorescence intensity of the fluorophore.

Embodiment 93. The method of Embodiment 91 or Embodiment 92, wherein the plurality of immobilized peptides are immobilized to the solid support via internal cysteine residues.

Embodiment 94. The method of any one of Embodiments 91-93, wherein the detecting comprises measuring an intensity of light emitted from the fluorophore.

Embodiment 95. The method of any one of Embodiments 91-94, wherein d) comprises repeating b) and c) at least two times.

Embodiment 96. The method of any one of Embodiments 91-95, wherein an N-terminal amino acid of an immobilized peptide of the plurality of immobilized peptides is of the amino acid type, wherein the immobilized peptide comprises at least one amino acid of the amino acid type separate from the N-terminal amino acid, and wherein in b) the N-terminal amino acid is removed.

Embodiment 97. The method of any one of Embodiments 91-96, wherein a pattern of degradation that coincides with a reduction of signal emitted by the fluorophore is unique to at least one peptide of the plurality of immobilized peptides.

Embodiment 98. The method of Embodiment 97, wherein the pattern is compared to a proteome of an organism to identify the at least one peptide.

Embodiment 99. The method of any one of Embodiments 91-98, further comprising, prior to b), contacting the plurality of immobilized peptides with an additional polymer of any one of Embodiments 1-54 under conditions sufficient to attach an additional polymer on amino acids of another amino acid type in the plurality of immobilized peptides.

Embodiment 100. The method of any one of Embodiments 91-99, wherein all amino acids of the amino acid type in the plurality of immobilized peptides comprise the polymer.

Embodiment 101. The method of any one of Embodiments 91-100, further comprising, prior to a), (i) providing a sample comprising a plurality of peptides, (ii) contacting the plurality of peptides with a polymer of any one of Embodiments 1-54 under conditions sufficient to attach the polymer to the amino acids of the amino acid type, and (iii) immobilizing the plurality of peptides on the solid support, thereby providing the plurality of immobilized peptides.

Embodiment 102. The method of any one of Embodiments 91-101, wherein the amino acid type is cysteine.

Embodiment 103. The method of any one of Embodiments 91-101, wherein the amino acid type is histidine.

Embodiment 104. The method of any one of Embodiments 91-101, wherein the amino acid type is tyrosine.

Embodiment 105. The method of any one of Embodiments 91-104, wherein the Edman degradation agent is an isothiocyanate derivative selected from the group consisting of phenyl isothiocyanate, fluorescein isothiocyanate, cyanine isothiocyanate, and rhodamine isothiocyanate.

Embodiment 106. The method of any one of Embodiments 91-105, wherein in c) an absence or a reduction in signal intensity indicates that the polymer has been removed.

Embodiment 107. A method, comprising:
a) providing a peptide immobilized on a solid support, wherein the peptide comprises at least two different types of amino acids coupled to at least two different types of the polymer of any one of Embodiments 1-54;
b) subjecting the peptide to conditions sufficient to remove a terminal amino acid of the peptide; and
c) detecting the at least two different types of polymer on the at least two different types of amino acids to sequence the peptide.

Embodiment 108. The method of Embodiment 107, wherein the at least two different types of amino acids comprise lysine.

Embodiment 109. The method of Embodiment 107 or Embodiment 108, wherein the at least two different types of amino acids comprise a carboxylic acid side chain.

Embodiment 110. The method of any one of Embodiments 107-109, wherein the at least two different types of amino acids comprise aspartic acid.

Embodiment 111. The method of any one of Embodiments 107-110, wherein the at least two different types of amino acids comprise glutamic acid.

Embodiment 112. The method of any one of Embodiments 107-111, wherein the peptide is immobilized on the solid support via cysteine residues.

Embodiment 113. The method of any one of Embodiments 107-112, wherein the terminal amino acid is a N-terminal amino acid.

Embodiment 114. The method of any one of Embodiments 107-113, wherein the terminal amino acid is a C-terminal amino acid.

Embodiment 115. The method of any one of Embodiments 107-114, wherein the terminal amino acid of the peptide is removed by an enzyme.

Embodiment 116. The method of Embodiment 115, wherein the enzyme comprises an Edman degradation agent.

Embodiment 117. The method of Embodiment 116, wherein the Edman degradation agent is an isothiocyanate derivative selected from the group consisting of phenyl isothiocyanate, fluorescein isothiocyanate, cyanine isothiocyanate, and rhodamine isothiocyanate.

Embodiment 118. The method of any one of Embodiments 107-117, wherein the detecting comprises measuring a fluorescence intensity of each of the fluorophores conjugated to the at least two different types of polymer.

Embodiment 119. The method of any one of Embodiments 107-118, wherein at least a portion of an emission spectra of each of the fluorophores conjugated to at least two different types of polymer do not overlap with one another.

Embodiment 120. The method of any one of Embodiments 107-119, wherein, in c), a reduction in signal intensity indicates that at least one amino acid of the at least two different types of amino acids coupled to the at least two different types of the polymer has been removed.

Embodiment 121. The method of any one of Embodiments 107-120, wherein, in c), an absence in signal intensity indicates that the at least two different types of amino acids coupled to the at least two different types of the polymer have been removed.

Embodiment 122. The method of anyone of Embodiments 107-121, where dye quenching between a first fluorophore conjugated to a first polymer of the at least two different types of polymer and a second fluorophore conjugated to a second polymer of the at least two different types of polymer is reduced compared to dye quenching between identical fluorophores conjugated directly to the at least two different types of amino acids.

Embodiment 123. The method of Embodiment 122, wherein the first fluorophore is Atto647N and the second fluorophore is Atto647N.

Embodiment 124. The method of anyone of Embodiments 107-121, where Förster resonance energy transfer (FRET) between a first fluorophore conjugated to a first polymer of the at least two different types of polymer and a second fluorophore conjugated to a second polymer of the at least two different types of polymer is reduced compared to FRET between identical fluorophores conjugated directly to the at least two different types of amino acids.

Embodiment 125. The method of Embodiment 124, wherein the first fluorophore is Atto647N and the second fluorophore is Janelia Fluor® 549.

Embodiment 126. The method of any one of Embodiments 107-125 further comprising, prior to b), contacting the peptide immobilized on the solid support with an additional polymer of any one of Embodiments 1-54 under conditions sufficient to couple the additional polymer to another type of amino acid different from the at least two different types of amino acids.

Embodiment 127. The method of any one of Embodiments 107-126, wherein the peptide comprises at least three different types of amino acids coupled to at least three different types of polymer of any one of Embodiments 1-54.

Embodiment 128. A method for identifying a sequence of a polypeptide, comprising:
a) providing the polypeptide;
b) contacting the polypeptide with a first polymer configured to couple with a first amino acid of the polypeptide;
c) contacting the polypeptide with a second polymer configured to couple with a second amino acid of the polypeptide;
d) immobilizing the polypeptide directly or indirectly to a support;
e) subjecting the polypeptide to conditions sufficient to remove at least one amino acid from the polypeptide;
f) detecting a signal or a signal change associated with the first polymer or the second polymer from the polypeptide; and
g) identifying, using at least one of the signal or the signal change, at least a portion of the sequence of the polypeptide;
wherein the first amino acid has greater nucleophilicity than the second amino acid;
wherein step b) occurs before step c); and
wherein the first and the second polymer are the polymer of any one of Embodiments 1-54.

Embodiment 129. The method of Embodiment 128, wherein:
a) the first amino acid comprises cysteine and the second amino acid comprises lysine; or
b) the first amino acid comprises cysteine and the second amino acid comprises glutamic acid and aspartic acid; or
c) the first amino acid comprises tyrosine and the second amino acid comprises glutamic acid and aspartic acid.

Embodiment 130. The method of Embodiment 128 or 129, wherein the at least one amino acid is removed from an N-terminus of the polypeptide.

Embodiment 131. The method of any one of Embodiments 128-130, wherein the first amino acid or the second amino acid comprises a plurality of amino acids, and wherein the at least one signal or signal change comprises a collective signal from the polypeptide and associated with a plurality of first polymers or a plurality of second polymers coupled thereto.

Embodiment 132. The method of any one of Embodiments 128-131, wherein the first polymer and the second polymer generate different signals or signal changes.

Embodiment 133. The method of anyone of Embodiments 128-132, wherein the signal or the signal change comprises a plurality of signals of different intensities.

Embodiment 134. The method of anyone of Embodiments 128-133, wherein the signal or the signal change is detected with an optical detector having single-molecule sensitivity.

Embodiment 135. The method of anyone of Embodiments 128-134, wherein the first polymer is configured to covalently couple to the first amino acid and the second polymer is configured to covalently couple to the second amino acid.

Embodiment 136. The method of anyone of Embodiments 128-135, wherein step b) occurs before step d).

Embodiment 137. The method of anyone of Embodiments 128-136, where dye quenching between a fluorophore conjugated to the first polymer and a fluorophore conjugated to the second polymer is reduced compared to dye quenching between identical fluorophores conjugated directly to the first amino acid and the second amino acid.

Embodiment 138. The method of Embodiment 137, wherein the fluorophore conjugated to the first polymer is Atto647N and the fluorophore conjugated to the second polymer is Atto647N.

Embodiment 139. The method of anyone of Embodiments 128-136, where Förster resonance energy transfer (FRET) between a fluorophore conjugated to the first polymer and a fluorophore conjugated to the second polymer is reduced compared to FRET between identical fluorophores conjugated directly to the first amino acid and the second amino acid.

Embodiment 140. The method of Embodiment 139, wherein the fluorophore conjugated to the first polymer is Atto647N and the fluorophore conjugated to the second polymer is Janelia Fluor® 549.

Embodiment 141. A method of making a polymer of any one of Embodiments 1-54, the method comprising:
(a) synthesizing a peptide of a sequence Fmoc-flexible spacer-rigid polypeptide-amino acid residue (boc)-CONH₂ using a solid phase peptide synthesizer;
(b) removing the Fmoc group and conjugating a functional group to a first end of the polymer; and
(c) conjugating a fluorophore to a second end of the polymer via the amino acid residue.

Embodiment 142. The method of Embodiment 141, wherein the amino acid residue is a lysine residue.

Embodiment 143. The method of Embodiment 141 or Embodiment 142, wherein the functional group is a click-reactive group.

Embodiment 144. The method of anyone of Embodiments 141-143, wherein the sequence Fmoc-flexible spacer-rigid polypeptide-amino acid residue (boc)-CONH₂ is Fmoc-Gly-(O—CH₂—CH₂)₂-Gly-Pro₃₀-Lys(boc)-CONH₂.

Embodiment 145. The method of anyone of Embodiments 141-144, wherein the functional group is DBCO, wherein the DBCO is conjugated via a DBCO-NHS molecule.

Embodiment 146. The method of anyone of Embodiments 141-145, wherein the fluorophore is Atto643, wherein the Atto643 is conjugated via a Atto643-NHS ester molecule.

Embodiment 147. A method for labeling an amino acid of a peptide, the method comprising:
(a) providing the peptide, wherein the peptide comprises an internal amino acid coupled to an azide and a C-terminus coupled to an alkyne; and
(b) bringing the peptide in contact with a first polymer of any one of Embodiments 1-54 under conditions such that the first polymer reacts with the internal amino acid, wherein the first polymer comprises a functional group that is a strained alkyne.

Embodiment 148. The method of Embodiment 147, wherein (b) is performed in the absence of copper (Cu).

Embodiment 149. The method of Embodiment 147 or Embodiment 148, further comprising, (c) reacting a second polymer of any one of Embodiments 1-54 different from the first polymer with said C-terminus, wherein the second polymer comprises a functional group that is a non-strained alkyne.

Embodiment 150. The method of Embodiment 149, wherein (c) is performed in the presence of copper (Cu).

Embodiment 151. The method of any one of Embodiments 147-150, wherein the azide coupled to the internal amino acid does not react with the alkyne coupled to the C-terminus.

Embodiment 152. A method for labelling an amino acid of a peptide, the method comprising:
(a) incubating a polymer of any one of Embodiments 1-54 with a peptide comprising an amino acid under conditions sufficient to react the functional group of the polymer with the peptide; and
(b) purifying the peptide.

Embodiment 153. The method of Embodiment 152, wherein the amino acid comprises azidolysine.

Embodiment 154. The method of Embodiment 152 or Embodiment 153, wherein the peptide comprises at least one lysine, and the method further comprising:
(c) functionalizing the lysine with NHS—(O—CH₂—CH₂)₄-azide; and
(d) incubating a second polymer of any one of Embodiments 1-54 with the peptide under conditions sufficient to react the functional group of the second polymer with the peptide.

Embodiment 155. The method of anyone of Embodiments 152-154, wherein the peptide is labeled with two or more polymers in a bottle brush configuration.

Embodiment 156. A kit for labeling an amino acid of a peptide, comprising:
(a) at least one polymer of any one of Embodiments 1-54, wherein the functional group of the polymer is configured to couple to an amino acid of an amino acid type;
(b) instructions for use to couple the polymer to the amino acid.

### EXAMPLES

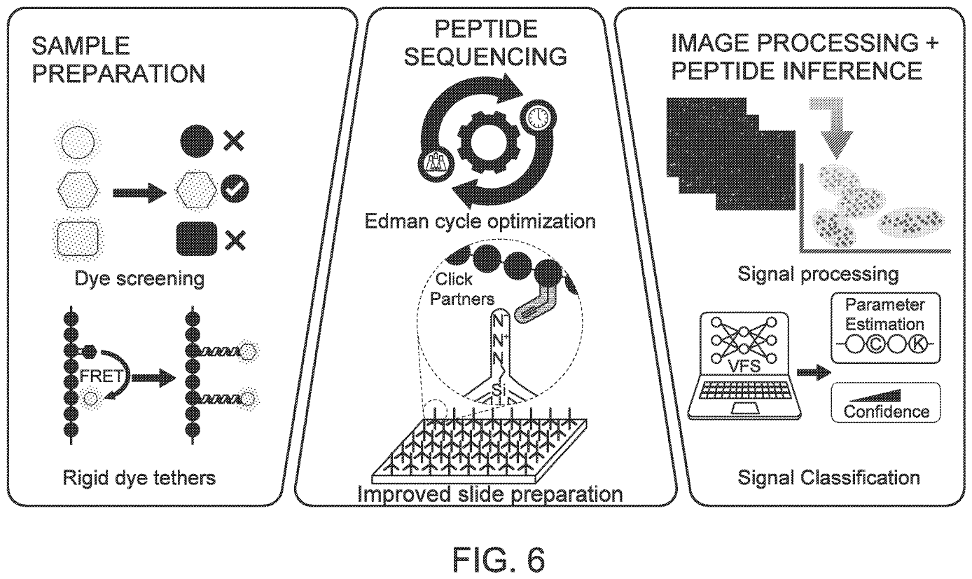

Improvements to fluorosequencing were made in three major areas: (a) the sample preparation, specifically the chemistry of labeling peptides, (b) peptide sequencing consisting of alternating cycles of Edman degradation and single molecule imaging and (c) the computational analysis, especially the image processing and peptide-read mapping. A glossary of terms as used in the experimental Examples and the peptides analyzed are provided in Table 2 and Table 3, respectively. FIG. 6 summarizes the changes and improvements to the process, which are described in detail in the Examples below.

TABLE 2
GLOSSARY OF TERMS

| Term | Definition |
|---|---|
| Aligned field stack | A set of images across cycles and channels for a |
| | given field, aligned such that peaks are in the |
| | same x-y positions in all cycles. |
| Alignment score | Maximum spatial shift in pixels after aligning |
| | images from the same field of view across all |
| | Edman cycles and all channels |
| Channel | Shorthand representation for an optical path with |
| | a defined laser/excitation and emission filter that |
| | produces an image, captured on the camera. |
| | Examples are the 647 channel and 561 channel. |
| Classifier | In the context of fluorosequencing, an algorithm |
| | used to perform peptide-read matching, i.e. to |
| | assign a read to a peptide sequence with an |
| | associated confidence score |
| Click | Bioorthogonal chemistry for coupling an azide to |
| | an alkyne. This reaction may or may not be |
| | copper-dependent. |
| Cycle | One round of Edman degradation. The term often |
| | denotes the set of images captured after an |
| | Edman degradation cycle, and may be called |
| | “Imaging cycle” to differentiate from an |
| | experimental Edman cycle. Cycle numbers begin |
| | from 0. |
| Dye sequence | The expected or inferred positions and identities |
| | of labeled amino acids, i.e. partial amino acid |
| | sequences |
| Dye track | The counts of each dye per sequence position. |
| | Both simulated and expected dye tracks will |
| | usually reflect errors such as photobleaching or |
| | Edman failures, which alter dye tracks relative to |
| | the ideal case. |
| Field | A region within the imageable portion of the flow |
| | cell with a fixed x, y coordinate. Fields are |
| | represented as numbers and begin from 0. |
| Field quality score | Score assigned to a field stack based on the |
| | number and size of visible fluorescent anomalies |
| | in its associated processed images |
| Field stack | The set of images for a given field and |
| | fluorescent channel collected across Edman |
| | cycles |
| Image | Captured fluorescence emission on a camera; |
| | represents one field of view for one fluorescent |
| | channel and one Edman cycle. |
| Intensity | The raw pixel value as reported by the camera |
| Kernel | Convolution kernel used to approximate the point |
| | spread function for a particular microscope, |
| | camera, and optical setup; used for |
| | computationally identifying fluorescent peaks in |
| | images (see Supplemental Note). |
| Peak | An image spot (occupying multiple camera |
| | pixels) capturing fluorescence from a labeled |
| | peptide |
| Point spread function | Normalized light intensity distribution from a |
| (PSF) | single point source emitter |
| Radiometry | Processed signal of a peak after applying |
| | background subtraction and regional balance, in |
| | arbitrary units |
| Radiometry matrix | A matrix such that each row is a peak and each |
| (radmat) | column is a measurement of brightness for each |
| | channel/cycle. May be stored in (n_peaks, |
| | n_channels, n_cycles) or flattened as (n_peaks, |
| | n_channels * n_cycles) |
| Read (raw sequencing | Post-signal processing radiometry data measured |
| read) | for an individual molecule across Edman cycles |
| | and fluorescent channels, in arbitrary fluorescent |
| | units/channel/cycle |
| Remainder | Fluorescent peak whose signal does not drop to |
| | background by the end of the sequencing run |
| Run parameters | Set of experimentally determined parameters used |
| | in simulating fluorosequencing process. Includes |
| | system -wide parameters and fluorophore specific |
| | parameters |
| Sample preparation | Series of experimental manipulations for |
| | preparing fluorescently labeled peptides from |
| | proteins prior to sequencing |
| Sequencing | Workflow that includes immobilizing peptides to |
| | slides, performing repeated cycles of Edman |
| | degradation, and imaging of single molecules |
| Virtual | Computational simulation of the |
| fluorosequencing | fluorosequencing process for a set of known |
| (VFS) | proteins and as a function of a given set of |
| | parameters and error rates; VFS returns simulated |
| | sequencing reads |

TABLE 3
PEPTIDES ANALYZED

| Pep | Purification | | |
|---|---|---|---|
| ID | method | Sequence | |
| JSP254 | HPLC | fmoc- | F = Atto643-NHS |
| | | AGK[F]GAAGGAANSYRN{PRA}R | |
| | | N | |
| | | (SEQ ID NO: 2) | |
| JSP263 | HPLC | fmoc- | F = Atto643-NHS |
| | | APK[F]GAAGGAANSYRN{PRA}R | |
| | | N | |
| | | (SEQ ID NO: 3) | |
| JSP264 | HPLC | fmoc- | F = Atto643-NHS |
| | | GAK[F]GAAGGAANSYRN{PRA}R | |
| | | N | |
| | | (SEQ ID NO: 4) | |
| JSP265 | HPLC | fmoc- | F = Atto643-NHS |
| | | AVK[F]GAAGGAANSYRN{PRA}R | |
| | | N | |
| | | (SEQ ID NO: 5) | |
| JSP266 | HPLC | fmoc- | F = Atto643-NHS |
| | | AAK[F]GAAGGAANSYRN{PRA}R | |
| | | N | |
| | | (SEQ ID NO: 6) | |
| JSP216 | Gel | Fmoc- | F = Atto643-Pro30-DBCO (LNK009) |
| | Extracted | G{azK}[F]GAA{azK}[F]G{azK}[F]A | |
| | | NSYRN{PRA}RN-COOH | |
| | | (SEQ ID NO: 7) | |
| JSP212 | | Fmoc- | F1 = NHS-N3, DBCO-P30-JF549; F2 = |
| | | GK[F1]{azK}[F2]AGAY{Pra}R- | DCBO-P30-Atto643 |
| | | COOH | |
| | | (SEQ ID NO: 8) | |
| JSP234 | Gel | NH2- | F1 = DBCO-P30-Atto643 |
| | Extracted | G{azK}[F1]AG{azK}[F1]AGAY | |
| | | {PRA}R-CONH2 | |
| | | (SEQ ID NO: 9) | |
| JSP251 | Gel | Fmoc- | F1 = NHS-PEG4-N3, DBCO-P30- |
| | Extracted | GK[F1]GAS{azK}[F2]GANSAK[F1] | Atto643; F2 = DBCO-P30-Alexa555 |
| | | GSYRN{PRA}R-CONH2 | |
| | | (SEQ ID NO: 10) | |
| JSP249 | Gel | Fmoc- | F1 = DBCO-Pro30-Alexa 555, NHS- |
| | Extracted | GK[F1]{azK}[F2]AGAY{Pra}R- | PEG4-N3; F2 = DBCO-Pro30-Atto643 |
| | | COOH | |
| | | (SEQ ID NO: 11) | |
| JSP252 | Gel | Fmoc- | F1 = DBCO-Pro30-Atto643, NHS- |
| | Extracted | GK[F1]AG{azK}[F2]AGSY{PRA}R- | PEG4-N3; F2 = DBCO-Pro30-Alexa555 |
| | | CONH2 | |
| | | (SEQ ID NO: 12) | |
| JSP248 | Gel | NH2- | F = DBCO-Pro30-Alexa555 (LNK033) |
| | Extracted | G{azK}[F]AG{azK}[F]AGAY{PRA} | |
| | | R-CONH2 | |
| | | (SEQ ID NO: 13) | |
| JSP308 | Gel | Fmoc- | F1 = DBCO-Pro30-TxRed; F2 = NH2- |
| | Extracted | E[F2]LYAE[F2]{azK}[F1]VATR- | Azide; DBCO-Pro30-Atto643 |
| | | {PRA}-G-CONH2 | |
| | | (SEQ ID NO: 14) | |
| JSP260 | HPLC | ac- | F = TexasRed-NHS |
| | | AK[F]GAAGGAANSYRN{PRA}RN- | |
| | | CONH2 | |
| | | (SEQ ID NO: 15) | |
| JSP288 | HPLC | ac- | F = Atto643-NHS |
| | | AK[F]GAAGGAANSYRN{PRA}RN | |
| | | (SEQ ID NO: 16) | |
| JSP127 | HPLC | Fmoc- | F = Atto643-NHS |
| | | AK[F]GAAGGAANSYRN{PRA}RN- | |
| | | CONH2 | |
| | | (SEQ ID NO: 17) | |
| JSP254 | HPLC | Fmoc- | F = Atto643-NHS |
| | | AGK[F]GAAGGAANSYRN{PRA}R | |
| | | N-CONH2 | |
| | | (SEQ ID NO: 18) | |
| JSP263 | HPLC | Fmoc- | F = Atto643-NHS |
| | | APK[F]GAAGGAANSYRN{PRA}R | |
| | | N-CONH2 | |
| | | (SEQ ID NO: 19) | |
| JSP264 | HPLC | Fmoc- | F = Atto643-NHS |
| | | GAK[F]GAAGGAANSYRN{PRA}R | |
| | | N-CONH2 | |
| | | (SEQ ID NO: 20) | |
| JSF265 | HPLC | Fmoc- | F = Atto643-NHS |
| | | AVK[F]GAAGGAANSYRN{PRA}R | |
| | | N-CONH2 | |
| | | (SEQ ID NO: 21) | |
| JSP266 | HPLC | Fmoc- | F = Atto643-NHS |
| | | AAK[F]GAAGGAANSYRN{PRA}R | |
| | | N-CONH2 | |
| | | (SEQ ID NO: 22) | |
| JSP285 | HPLC | Fmoc- | F = NHS-Atto643 |
| | | APAK[F]GAAGGAANSYRN{PRA} | |
| | | RN-CONH2 | |
| | | (SEQ ID NO: 23) | |
| JSP287 | HPLC | Fmoc- | F = NHS-Atto643 |
| | | AAPK[F]GAAGGAANSYRN{PRA} | |
| | | RN-CONH2 | |
| | | (SEQ ID NO: 24) | |
| JSP286 | HPLC | Fmoc- | F = NHS-Atto643 |
| | | PAK[F]GAAGGAANSYRN{PRA}R | |
| | | N-CONH2 | |
| | | (SEQ ID NO: 25) | |
| JSP146 | HPLC | Fmoc-GK[F]GSK[F]SYN{PRA}R- | F = Atto643-NHS |
| | | COOH | |
| | | (SEQ ID NO: 26) | |
| JSP162 | HPLC | Fmoc- | F = Atto643-NHS |
| | | AK[F]GAAK[F]GK[F]ANSYRN{PR | |
| | | A}RN-CONH2 | |
| | | (SEQ ID NO: 27) | |
| JSP333 | HPLC | Fmoc- | F = Atto643-NHS |
| | | AK[F]GK[F]AK[F]GK[F]ANSYRN | |
| | | {PRA}RN | |
| | | (SEQ ID NO: 28) | |
| JSP129 | HPLC | Fmoc- | F1 = JF549-NHS; F2 = Atto647N- |
| | | GK[F1]AGC[F2]AGAYG[Alkyne]R- | Iodoacetamide |
| | | COOH | |
| | | (SEQ ID NO: 29) | |
| JSP150 | HPLC | Fmoc-GK[F1]AGC[F2]AG{PRA}R- | F1 = Alexa 488-NHS; F2 = |
| | | COOH | Atto647N-Iodoacetamide |
| | | (SEQ ID NO: 30) | |
| JSP154 | HPLC | Fmoc- | F1 = JF525-NHS; F2 = Atto647N- |
| | | GK[F1]AGC[F2]AGAY{PRA}R- | Iodoacetamide |
| | | COOH | |
| | | (SEQ ID NO: 31) | |
| JSP168 | HPLC | H2N-K[F1]G{azLys}[F2]G{PRA}G- | F1 = TMR-G-PPP-; F2=Atto647N- |
| | | CONH2 | PEG4-DBCO |
| | | (SEQ ID NO: 32) | |
| YPep | HPLC | [F1]K[F2]G | F1 = TMR-G-P14 -; F2 = Atto647N- |
| | | | G-P14- |
| JSP213 | HPLC | Fmoc- | F1 = JF549-NHS; F2 = Atto643-PEG4- |
| | | GK[F1]{azK}[F2]AGAY{Pra}R- | DBCO |
| | | COOH | |
| | | (SEQ ID NO: 33) | |
| JSP214 | HPLC | Fmoc- | F1 = JF549-NHS; F2 = Atto643-P30- |
| | | GK[F1]{azK}[F2]AGAY{Pra}R- | DBCO |
| | | COOH | |
| | | (SEQ ID NO: 33) | |
| JSP274 | HPLC | Fmoc- | F = NHS-PEG4-N3; DBCO-P30- |
| | | APK[F]GAAGGAANSYRN{PRA}R | Atto643 |
| | | N-CONH2 | |
| | | (SEQ ID NO: 34) | |

### Example 1

### Expanding the Set of Fluorescent Dyes Compatible with Fluorosequencing

In this Example, fluorophores were identified that are stable across the chemical solvent and exhibit high brightness for single molecule TIRF experiments. Based on computational simulations of fluorosequencing (Swaminathan, Boulgakov, and Marcotte (2015) PLOS Computational Biology 11 (2): e1004080), the identification of proteins in complex samples generally entails selectively labeling 3 or 4 amino acid types, each with a different fluorophore. It has previously been observed that commonly used fluorophores, such as BODIPY and cyanine dyes, do not recover fluorescence after exposure to the solvents and reagents used in Edman sequencing chemistry, limiting the number of distinct fluorescent labels available for sequencing (Swaminathan et al. (2018) Nat. Biotechnol. 36, 1076-1082). Therefore, to identify additional fluorophores stable to Edman sequencing chemistry, a screen of 70 fluorescent dyes was conducted, spanning both commercially available and custom-synthesized dyes, and found 31 with the stability needed in important Edman reagents. For this screen, the succinimidyl variants of dyes were immobilized on amine-functionalized Tentagel beads and the fluorescence recovery was measured after exposure to methanol, trifluoroacetic acid, or 20% phenylisothiocyanatein pyridine. Stability in piperidine solution was also tested to ensure compatibility with synthetic peptides, which often need removal of an N-terminal Fmoc blocking group prior to fluorosequencing. Table 4 provides the solvent stability data for each of the fluorophores tested. It was observed that rhodamine and carbopyronine dyes generally showed superior solvent stability.

TABLE 4
FLUOROPHORE SOLVENT STABILITY

| Legend | | |
|---|---|---|
| Recovery Range | Assignment | Manual Assignment |
| Plus/Minus (0 to 20) % | Good | Good |
| Plus/Minus (20-50)% | Maybe | Maybe good/maybe bad |
| Plus/Minus (>50)% | Bad | Bad |

| | Recovery of fluorescence after 24 h solvent exposure | | | | | |
|---|---|---|---|---|---|---|
| | | | | 20% | | |
| | | | | PITC in | Manual | |
| Fluorophore | TFA | Base | Piperidine | Pyridine | Assignment | Comments |
| Janelia Fluor 549 | Maybe | Maybe | Maybe | Good | Good | recover in |
| (JF549) | | | | | | all solvents |
| Janelia Fluor 525 | Bad | Bad | Bad | Good | Bad | Didn't |
| (JF525) | | | | | | recover |
| Janelia Fluor 479 | Bad | Bad | Bad | Good | Bad | Didn't |
| (JF479) | | | | | | recover |
| JFX 554 | Good | Good | Good | Good | Good | recover in |
| | | | | | | all solvents |
| JFX 650 | Good | Good | Good | Good | Good | recover in |
| | | | | | | all solvents |
| CF 568 | Good | Maybe | Maybe | Good | Good | recover in |
| | | | | | | all solvents |
| CF 633 | Good | Bad | Bad | Good | Bad | Didn't |
| | | | | | | recover |
| | | | | | | base |
| CF640R | Good | Bad | Bad | Good | Bad | Didn't |
| | | | | | | recover |
| | | | | | | base |
| CF680R | Good | Good | Bad | Bad | Bad | Didn't |
| | | | | | | recover |
| | | | | | | base |
| Atto390 | Good | Good | Good | Good | Good | recover in |
| | | | | | | all solvents |
| Atto425 | Good | Good | Good | Good | Good | recover in |
| | | | | | | all solvents |
| Atto488 | Bad | Bad | Good | Good | Bad | Didn't |
| | | | | | | recover |
| | | | | | | TFA |
| Atto514 | Good | Bad | Maybe | Good | Good | Error in |
| | | | | | | image |
| | | | | | | analysis; |
| | | | | | | Didn't have |
| | | | | | | ring in base |
| Atto495 | Good | Good | Good | Good | Good | recover in |
| | | | | | | all solvents |
| Atto647N | Good | Good | Good | Good | Good | recover in |
| | | | | | | all solvents |
| Atto643 | Good | Good | Good | Good | Good | recover in |
| | | | | | | all solvents |
| Atto532 | Good | Good | Good | Good | Good | recover in |
| | | | | | | all solvents |
| Atto647 | Bad | Maybe | Good | Good | Bad | Didn't |
| | | | | | | recover |
| | | | | | | acid |
| Atto655 | Good | Bad | Bad | Bad | Bad | Didn't |
| | | | | | | recover |
| Atto680 | Bad | Bad | Bad | Good | Bad | Didn't |
| | | | | | | recover |
| Atto700 | Bad | Bad | Bad | Good | Bad | Didn't |
| | | | | | | recover |
| Rhodamine 110 (5- | Bad | Good | Good | Bad | Bad | Didn't |
| CR110) | | | | | | recover |
| | | | | | | PITC |
| Rhodamine 6G | Good | Good | Good | Bad | Bad | Didin't |
| | | | | | | recover |
| | | | | | | PITC |
| Rhodamine B | Good | Good | Good | Good | Good | recover in |
| | | | | | | all solvents |
| Carboxyrhodamine B | Good | Good | Good | Good | Good | recover in |
| | | | | | | all solvents |
| Tetramethylrhodamine | Good | Good | Good | Good | Good | recover in |
| | | | | | | all solvents |
| Rhodamine 101 | Good | Good | Good | Good | Good | recover in |
| | | | | | | all solvents |
| Rhodamine Si | Good | Good | Good | Good | Good | recover in |
| | | | | | | all solvents |
| 5-carboxyfluorescein | Good | Good | Good | Good | Good | recover in |
| | | | | | | all solvents |
| Napthofluorescein | Good | Maybe | Good | Good | Good | Low |
| | | | | | | intensity |
| OG488 | Good | Good | Good | Good | Good | Low |
| | | | | | | intensity |
| OG514 | Good | Good | Good | Good | Good | Low |
| | | | | | | intensity |
| 6-JOE-N3 | Good | Good | Good | Good | Good | recover in |
| | | | | | | all solvents |
| 7-hydroxycoumarin- | Good | Bad | Bad | Good | Bad | Didn't |
| N3 | | | | | | recover |
| | | | | | | base |
| Cy3 | Maybe | Maybe | Bad | Bad | Bad | Didn't |
| | | | | | | recover |
| Cy5 | Bad | Bad | Bad | Good | Bad | Didn't |
| | | | | | | recover |
| Cy3B | Bad | Good | Bad | Good | Bad | Didn't |
| | | | | | | recover |
| | | | | | | acid |
| Cy5B | Bad | Good | Bad | Good | Bad | Didin't |
| | | | | | | recover |
| | | | | | | acid |
| Cy488 | Bad | Good | Bad | Good | Bad | Didn't |
| | | | | | | recover |
| | | | | | | acid |
| Dylight405 | Good | Good | Good | Maybe | Bad | Low |
| | | | | | | intensity; |
| | | | | | | Beyond |
| | | | | | | relevant |
| | | | | | | spectra |
| Dylight488 | Good | Good | Good | Bad | Bad | Didn't |
| | | | | | | recover |
| | | | | | | PITC |
| iFluor710 | Good | Bad | Bad | Good | Bad | Didn't |
| | | | | | | recover |
| | | | | | | base |
| Alexa Fluor 405 | Good | Good | Good | maybe | Good | Low |
| | | | | | | intensity; |
| | | | | | | Not ideal |
| | | | | | | laser |
| Alexa Fluor 488 | Bad | Good | Maybe | Bad | Bad | Didn't |
| | | | | | | recover |
| | | | | | | acid |
| Alexa Fluor 555 | Good | Maybe | Bad | Good | Good | recover in |
| | | | | | | all solvents |
| Alexa Fluor 647 | Bad | Maybe | Bad | Good | Bad | Didn't |
| | | | | | | recover |
| Alexa Fluor 680 | Good | Bad | Bad | Good | Bad | Didn't |
| | | | | | | recover |
| | | | | | | base |
| CF398 | Good | Good | Bad | Bad | Bad | Low |
| | | | | | | intensity |
| CF430 | Good | Good | Maybe | Good | Good | recover in |
| | | | | | | all solvents |
| DY350XL | Good | Good | Bad | Good | Bad | Didn't |
| | | | | | | recover |
| | | | | | | base |
| DY351XL | Good | Good | Good | Good | Good | Low |
| | | | | | | intensity |
| DY360XL | Bad | Good | Good | Good | Bad | Low |
| | | | | | | intensity |
| DY370XL | Good | Good | Bad | Maybe | Bad | Didn't |
| | | | | | | recover |
| | | | | | | base; Low |
| | | | | | | intensity |
| DY376XL | Good | Maybe | Maybe | Good | Bad | Didn't |
| | | | | | | recover |
| | | | | | | base |
| DY380XL | Good | Good | Bad | Good | Bad | Low |
| | | | | | | intensity |
| DY396XL | Good | Bad | Bad | Good | Bad | Low |
| | | | | | | intensity |
| DY720 | Bad | Bad | Good | Bad | Bad | Unavailable |
| | | | | | | filter set |
| Bodipy493 | Bad | Bad | Bad | Good | Bad | Didn't |
| | | | | | | recover |
| | | | | | | TFA |
| BodipyFL | Bad | Bad | Bad | Bad | Bad | Didn't |
| | | | | | | recover |
| Bodipy650 | Bad | Bad | Bad | Good | Bad | Didn't |
| | | | | | | recover |
| Pyrene-N3 | Bad | Good | Good | Good | Good | Increase in |
| | | | | | | TFA; But |
| | | | | | | didnt |
| | | | | | | activate |
| Lucifer Yellow | Bad | Good | Good | Good | Good | Didn't have |
| | | | | | | ring in TFA |
| Eterneon 394 | Good | Good | Good | Bad | Bad | Didn't |
| | | | | | | recover |
| | | | | | | PITC |
| Hilyte 405 | Bad | Maybe | Maybe | Maybe | Bad | Didn't |
| | | | | | | recover |
| | | | | | | acid |
| Hilyte 488 | Bad | Maybe | Maybe | Bad | Bad | Didn't |
| | | | | | | recover |
| | | | | | | acid |
| Hilyte 647 | Bad | Bad | Bad | Maybe | Bad | Didn't |
| | | | | | | recover |
| | | | | | | acid |

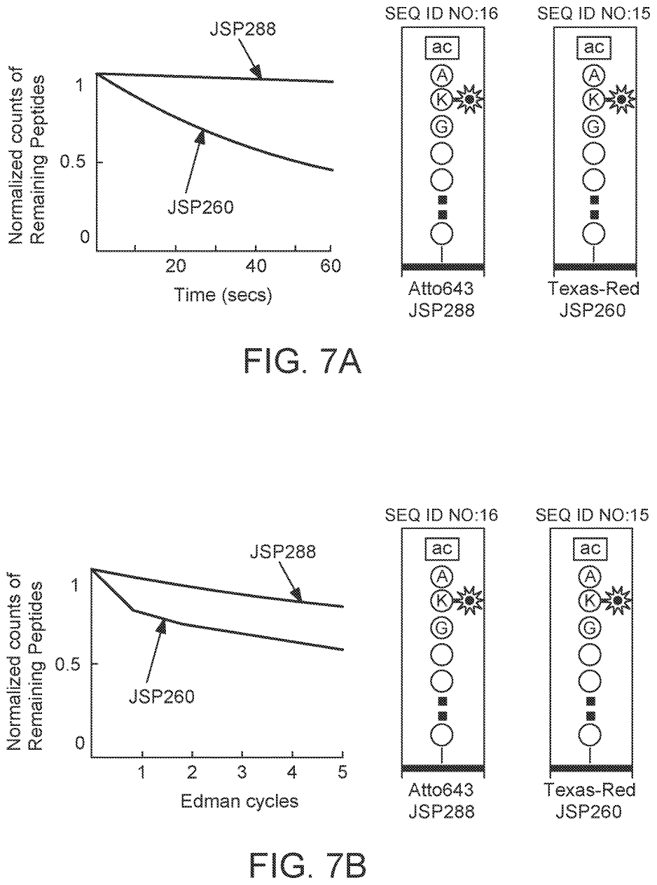

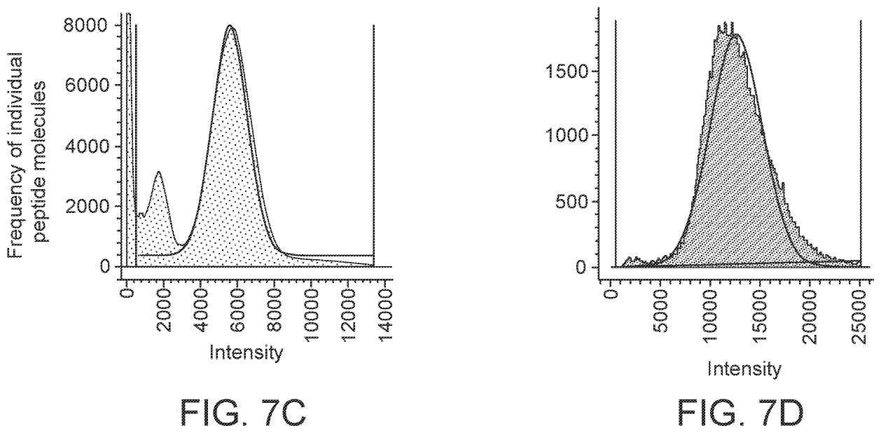

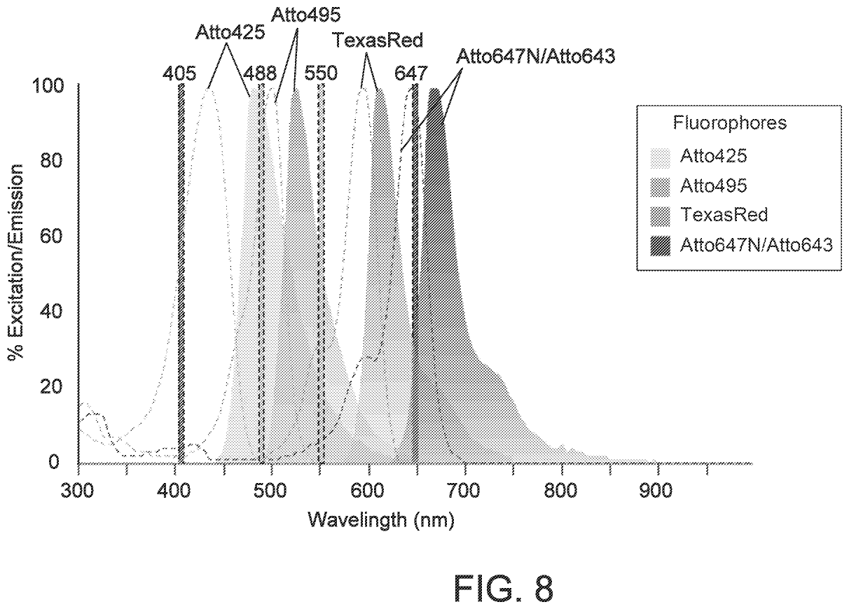

From this set of solvent-stable fluorophores, further dyes were prioritized based on their practical performance and imaging quality in fluorosequencing experiments. Four different experimental parameters were measured for the dyes, including their chemical destruction rate, the per cycle fluorescence loss of dyes on N-terminally acetylated (hence, not sequenceable) peptides after exposure to Edman reagents; photobleaching rate, the fluorescence loss rate during constant illumination in imaging solvent (0.1 mM Trolox in degassed methanol); and their fluorescence brightness and its variation. FIGS. 7A-7D report these parameters for two of the dyes selected, TexasRed and Atto643. Photobleaching and chemical destruction rates were measured for Atto643 at 1.9% and 2% per cycle, respectively, and TexasRed at 0.46% and 5.6% per cycle. These results demonstrate that TexasRed, Alexa555, and Atto643 may generally be used as a reasonable starting palette of fluorosequencing labels. Table 5 reports their relevant parameters, while FIG. 8 highlights four additional dyes that also appeared suitable for fluorosequencing experiments.

TABLE 5
EXPERIMENTALLY DETERMINED PARAMETERS FOR
FLUOROPHORES - ALEXA555, TEXASRED-X AND
ATTO643 FLUOROPHORE USED IN THE EXPERIMENTS

| | Parameters | | | |
|---|---|---|---|---|
| | Combined | | Mean | |
| | photobleach | Photo- | brightness | S.D. of |
| | and chemical- | bleaching | (approx, | intensity |
| | destruction rates | rate (% loss | arbitrary | distribution |
| Fluorophore | (% loss per cycle) | per cycle) | units) | (approx) |
| Alexa555 | 5.6 | 0.35 | 8300 | 0.16 |
| Texas Red - | 5.6 | 0.46 | 8900 | 0.23 |
| X | | | | |
| Atto643 | 2 | 0.17 | 12000 | 0.13 |

### Example 2

### Alkyne Modifications to Peptides Reduce Non-Specific Peptide Binding to the Silane Surface

In this example, peptide-slide attachment was modified through an azide-alkyne click reaction. An important factor to consider is that of how peptides are anchored in the flow cell for sequencing. Significant amounts of non-specific binding of labeled peptides to aminosilane-labeled glass surfaces had previously been observed, an effect attempted previously to be controlled for by fluorosequencing a negative control (N-terminally acetylated) peptides in parallel (Swaminathan et al. (2018) Nat. Biotechnol. 36, 1076-1082). It was speculated that non-specific interactions of either the peptide or the dye with the slide surface might be due to the charged amine groups of the aminosilane, and that by moving to an uncharged surface, such as by immobilizing peptides using azide-alkyne click chemistry, the observed nonspecific binding might be reduced. Glass slides were therefore functionalized with 3-azidopropyltriethoxysilane and modified peptides to contain C-terminal alkyl groups. For synthetic peptides, this was achieved through the use of Fmoc-propargylglycine as the C-terminal amino acid building block. After making this modification a 23-fold improvement in correctly sequenceable peptides was observed (Hinson et al. 2021 Langmuir: The ACS Journal of Surfaces and Colloids 37 (51): 14856-65). These results demonstrate that it is advantageous to immobilize peptides for fluorosequencing by azide-alkyne click chemistry.

### Example 3

### Changes to Edman Chemistry Increase the Efficiency and Speed and Reduce Dye Destruction Rates

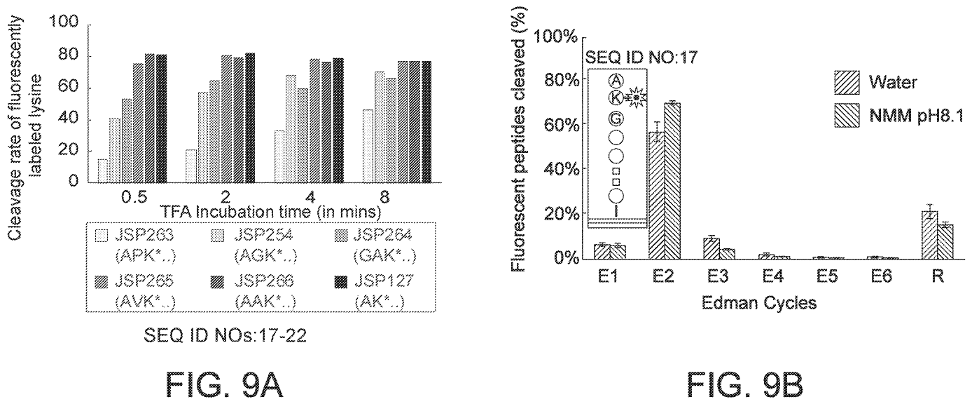

In this Example, improvements were made to Edman chemistry for greater reproducibility and efficiency. It was sought to make improvements to the slide preparation process and the Edman sequencing chemistry itself, and thus further reduce the rates of slide surface and dye destruction due to the sequencing chemistry (Smith and Chen (2008) Langmuir: The ACS Journal of Surfaces and Colloids 24 (21): 12405-9). First the role of incubation times of the various Edman reagents was explored. In particular, changing the TFA incubation times caused a dramatic improvement to the previously reported sequencing efficiency, as shown in FIG. 9A. The TFA incubation time was set to 8 mins per cycle, as longer incubation times begin to impact the chemical destruction of the fluorophores.

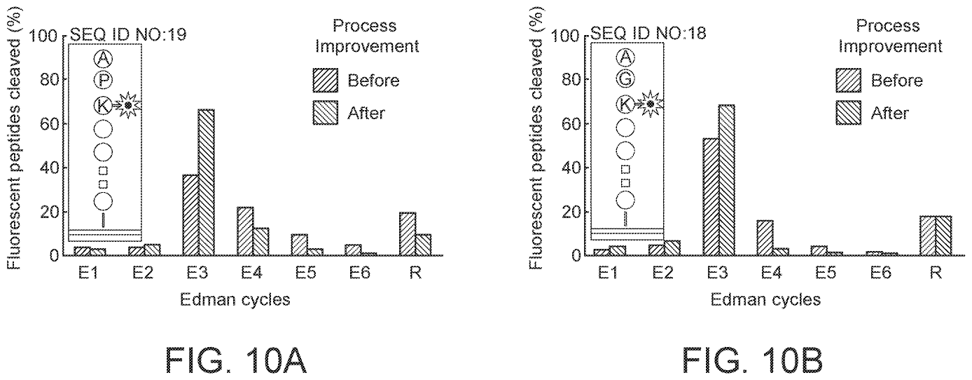

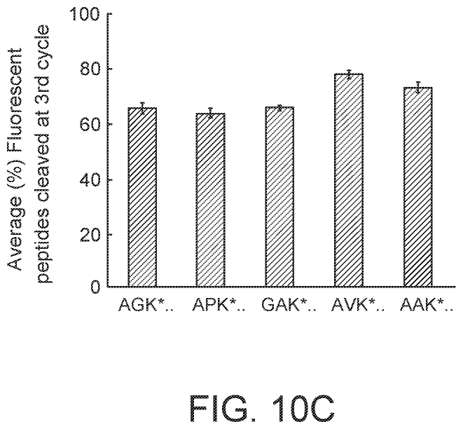

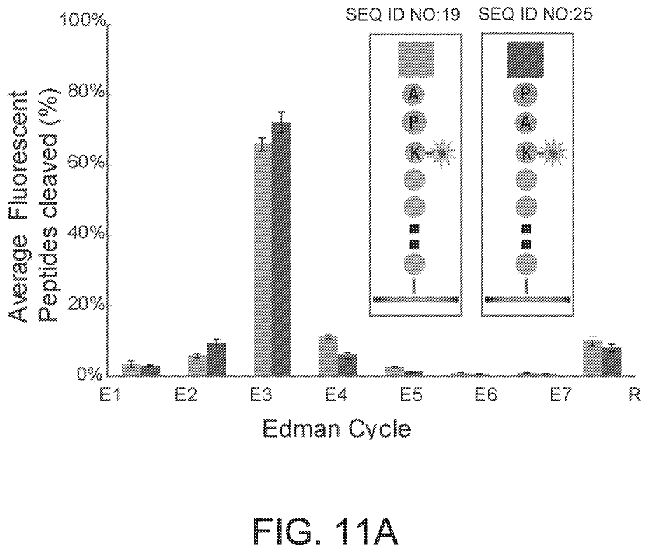

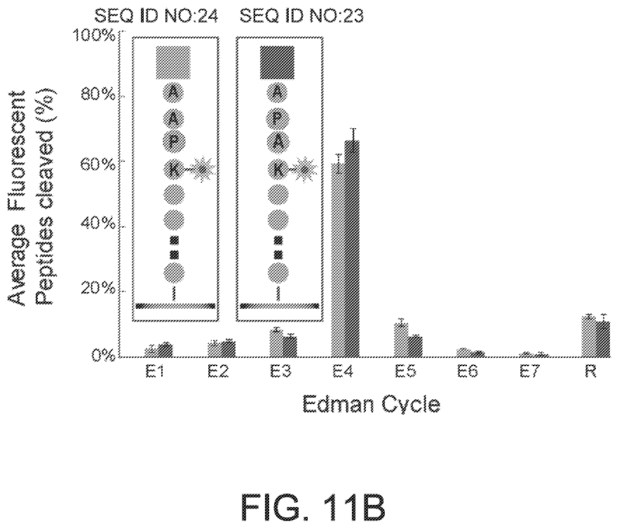

Similarly, it was found that doubling either the incubation time or concentration of the Edman reagent, phenylisothiocyanate, improved the efficiency; moving forward, 20% v/v in pyridine was used (twice the prior concentration) to avoid increasing reaction times. Additionally it was found that adding 60 mM of N-methylmorpholine base into the phenylisothiocyanate coupling solution and increasing the reaction temperature by 10° C. further increased the reaction efficiency (FIG. 9B). Applying these changes improved the % of peptides showing the expected fluorosequence (an easily measured metric that correlates with Edman efficiency) for a difficult to sequence proline-containing peptide (JSP263) from 35 to 67% (FIG. 10A), corresponding to ˜91-99% Edman efficiency. Similar improvements were seen across a wide variety of peptides of varying composition, as shown for several examples in FIG. 10C. In general, it was found that proline was the most resistant to Edman cleavage, consistent with historic observations (Brandt et al. 1976 Hoppe-Seyler's Zeitschrift Fur Physiologische Chemie 357 (11): 1505-8), with an average decrease of 9.5% in the percent of peptides successfully showing the largest fluorescent intensity decrease at the expected label position when the labeled residue is preceded by proline (FIGS. 11A-11B). The effect depended in part on the identity of the amino acid that follows the proline, likely due to the high energy barrier in forming the bicyclic of phenylthiohydantoin-proline during the Edman degradation mechanism (Tarr 1975 Analytical Biochemistry 63 (2): 361-70; Brandt et al. 1976 Hoppe-Seyler's Zeitschrift Fur Physiologische Chemie 357 (11): 1505-8). As a final improvement to the Edman sequencing process, the fluidic system was simplified by eliminating the free-base step and substituting ethyl acetate with acetonitrile. It was also observed that water in the base mix was important for successful Edman chemistry and purging with nitrogen has only limited benefits. These results demonstrate that, with all of these changes considered together, the full cycle of Edman chemistry was reduced to 40 minutes.

### Example 4

### Vapor Deposition of Silane Improves Consistency and Reduces Contamination on Flow Cell Surfaces

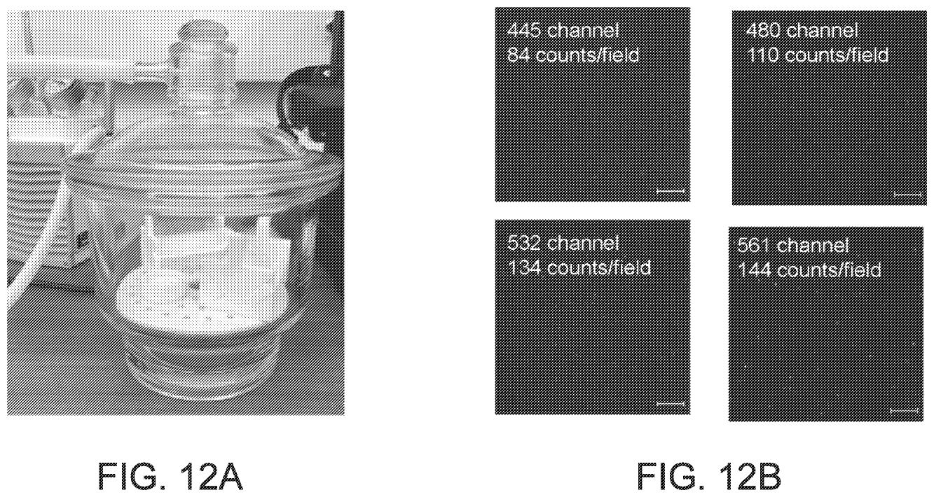

In this Example, a vapor deposition method for applying silane to a glass flow cell surface was developed. It was sought to improve the quality of surface preparations in the sequencing flow cells, as contaminating fluorescent molecules are often noted in early sequencing experiments. A vapor deposition method for applying silane to the glass flow cell surface was therefore implemented instead of the previously used dip coating method. This change resulted in a significant reduction of fluorescent contaminants in the 488, 532 and 561 nm imaging channels, which were ascribed to reduced handling and increased uniformity of the surfaces and across preparation batches due to reduced silane polymerization (Popat, Johnson, and Desai 2002 Surface and Coatings Technology 154 (2): 253-61). FIGS. 12A-12B illustrate the vapor deposition method and demonstrate the quality of the resulting surfaces.

### Example 5

### Development of Rigid Polyproline Polymers to Minimize Dye-Dye Interactions

In this Example, fluorophores were installed on a peptide backbone using long rigid linkers, called polymers, or “promers”. A polymer or “promer” as used in the Examples may be a polymeric linker containing 30 proline units with a fluorophore at the C-terminal end and a functional group (such as DBCO) with a flexible poly-Gly/PEG linker on the N-terminus.

### Background

As multiple numbers and types of fluorophores were installed on peptides, dye-dye interactions were increasingly observed that complicated deconstructing the observed fluorescent signals into their respective counts and types of fluorophores, an important interpretive step. Two different types of dye-dye interactions in particular were observed in the system-dye-dye quenching and Förster resonance energy transfer (FRET).

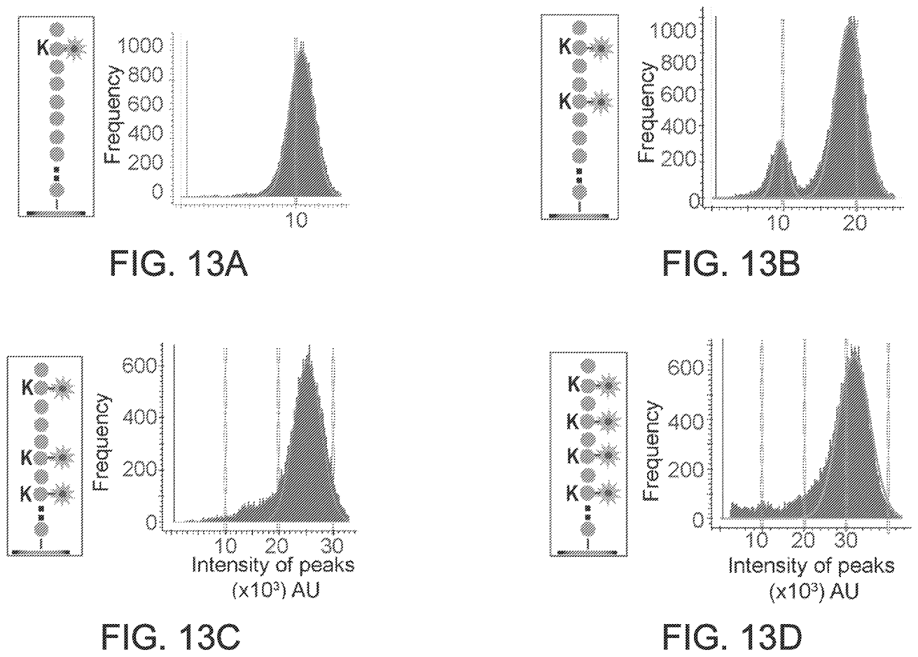

Dye-dye quenching was evident when multiple copies of the same Atto647N fluorescent dye were installed on the same peptide. For example, it was observed that the fluorescence intensity distribution of peptides containing either 1, 2, 3, or 4 Atto647N dyes did not follow a simple additive increase of single dye intensity distribution (FIGS. 13A-13D). This behavior could be attributed to dye quenching between closely spaced dyes.

In contrast, FRET was evident when imaging peptides with two different fluorophores, in which signals primarily from the higher wavelength emitter were observed. Prior to improving the sequencing chemistries and surfaces, earlier sequencing experiments showed relatively little evidence for such interactions; it is speculated that high dud-dye rates and non-specific binding of dyes to the surface conflated the signal. Moreover, FRET might also be unlikely due to the flexibility of the fluorophore linker attached to the peptide backbone and the use of organic solvents (methanol) for imaging which prohibits dye-dye interactions such as pi-stacking and H-dimer formation (Ogawa et al. (2009) ACS Chemical Biology 4 (7): 535-46). However, with the system-wide improvements made to reduce background contamination, an effect due to FRET was considerably more evident.

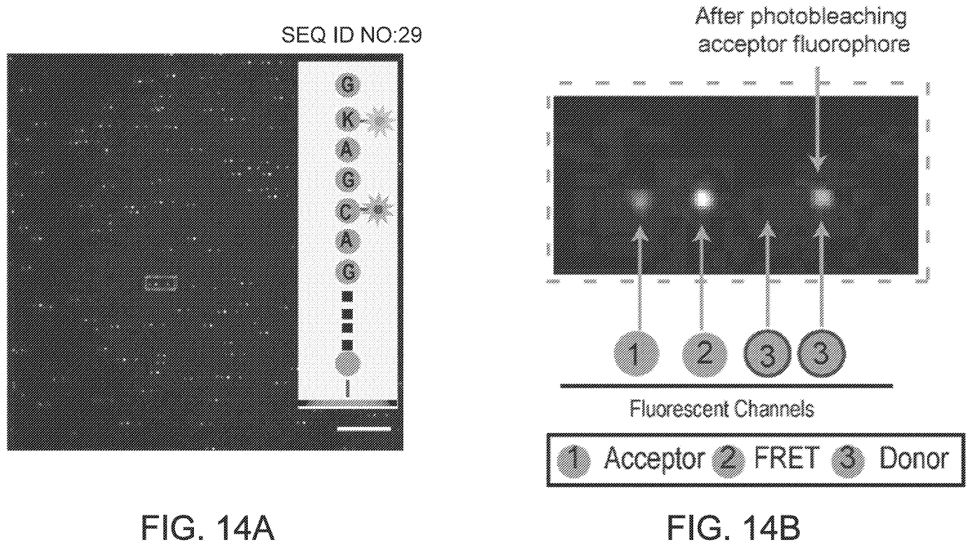

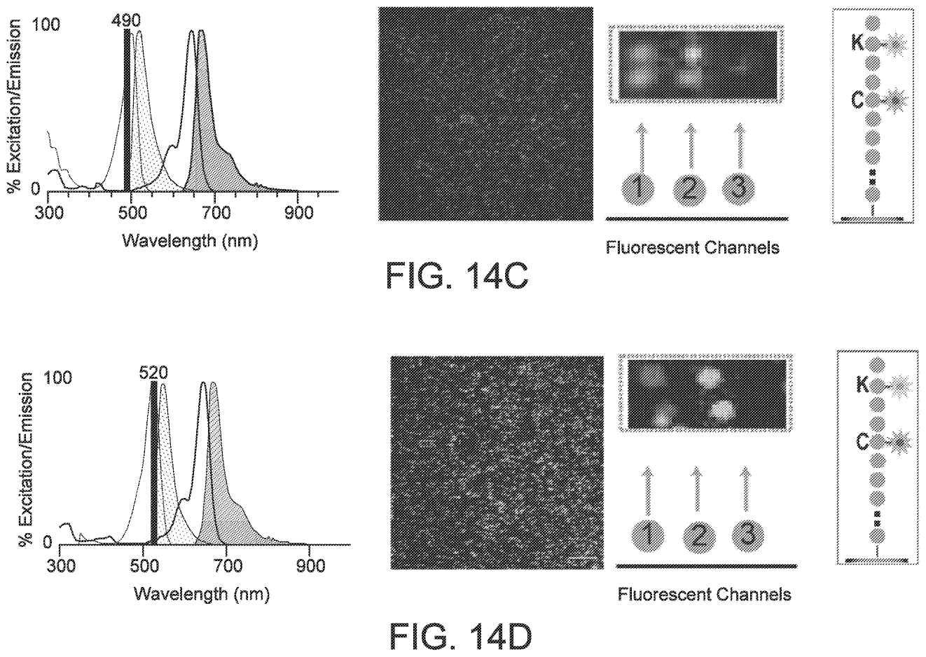

As an example of the FRET observed, FIG. 14A shows data for a peptide (JSP129) containing two distinct fluorophores, Janelia Fluor 549 and Atto647N, with less than 5% colocalization of peptide peaks from the two imaging channels. To explore if this observation resulted from FRET, fluorescent single molecules were imaged using the expected excitation and emission for each fluorophore and an additional “FRET channel”, exciting with the lower wavelength and measuring the higher wavelength emission. For a majority of the peptides, significant signals in the FRET channel and the Atto647N channel were observed; bleaching the Atto647N caused the Janelia Fluor 549 signal to return, confirming intramolecular dye-dye energy transfer. It was concluded that FRET occurred at a very high rate in this and other peptides, with a measured FRET efficiency of >90% for this pair of dyes. Similarly low colocalization and high FRET was observed between the dye pairs Alexa488/Atto647N and JF525/Atto647N (FIG. 14B). Thus, a major effort was made to try to mitigate both types of dye-dye interactions by introducing the appropriate linkers for attaching the dyes to the peptides, intended to position the dyes far enough apart to reduce their interactions.

### Rigid Polyproline Polymers Minimize Dye-Dye Interactions

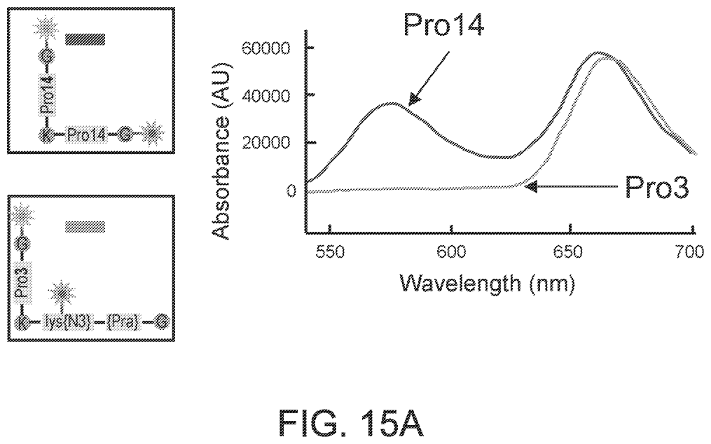

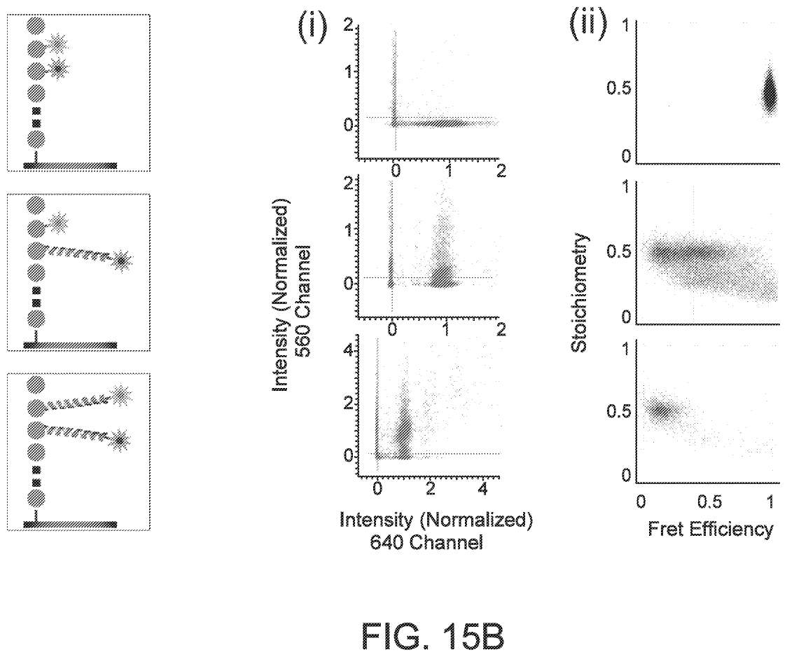

Previously, it was found that attaching fluorophores to peptides using flexible (PEG)₁₀ linkers provided a modest reduction of dye-dye quenching (Bachman et al. (2022) Bioconjugate Chemistry 33 (6): 1156-65). However, the inventors of the present disclosure suspected that the short length (approx. 30 Å) and high flexibility of these PEG polymers limited their effectiveness in spatially separating the fluorophores. To test this hypothesis a branched peptide with two 14 subunit polyproline peptides on the two arms was synthesized, each labeled with either TMR or Atto647N. The observation of a 39% recovery of the donor signal in this system (see FIG. 15A), when compared to a peptide system without the polyproline linker, indicated that spacing dyes through rigid polyproline linkers is a viable approach to mitigate dye-dye interactions.

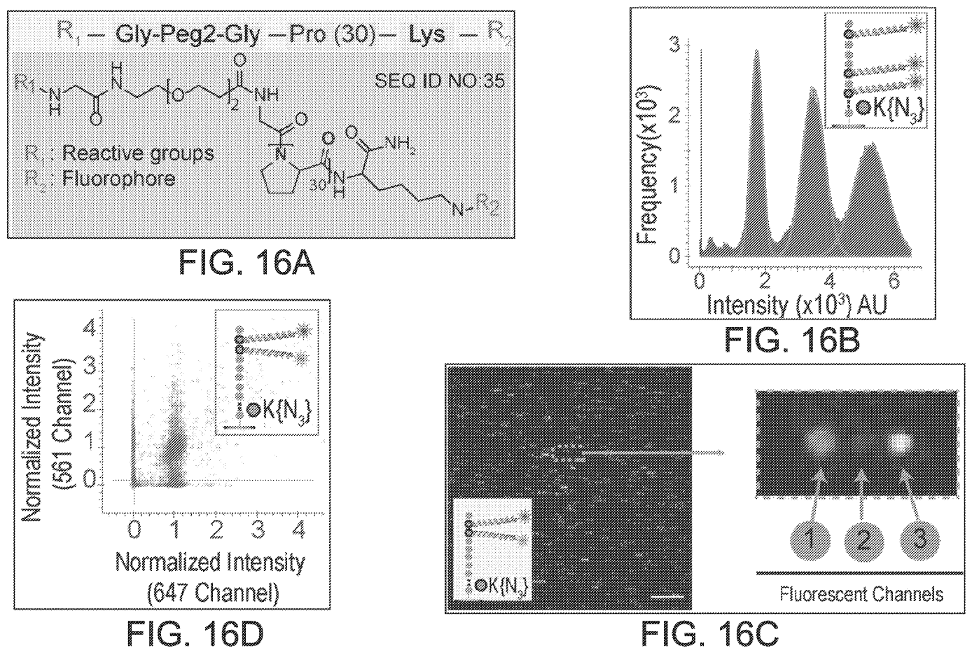

To explore this notion more fully, a number of different length proline linkers were tested and it was determined that a 30-proline linker significantly reduced FRET to less than 10% while still being synthetically accessible (FIG. 15B). In order to install these long polyproline linkers with fluorophores on peptides, peptidic linkers (termed “polymers” or “promers”) were designed and synthesized with the following characteristics: At the N-terminus, a “Gly-(PEG)₂-Gly” unit was synthesized to increase solubility and flexibility, followed by a 30-proline repeat. The terminal amine was labeled with a reactive chemical moiety, such as dibenzocyclooctyne (DBCO), and at the C-terminus, a lysine was synthesized, whose side chain could then be functionalized with the desired fluorophore (FIG. 16A). In general, polyproline is thought to organize as a rigid rod (Schuler et al. 2005 PNAS 102 (8): 2754-59; El-Baba et al. 2019 Journal of the American Society for Mass Spectrometry 30 (1): 77-84) whose length, for the case of the polymer and depending on the polarity of the solvent, is estimated to be between 6-9 nm, above the Förster radii of many fluorophore pairs.

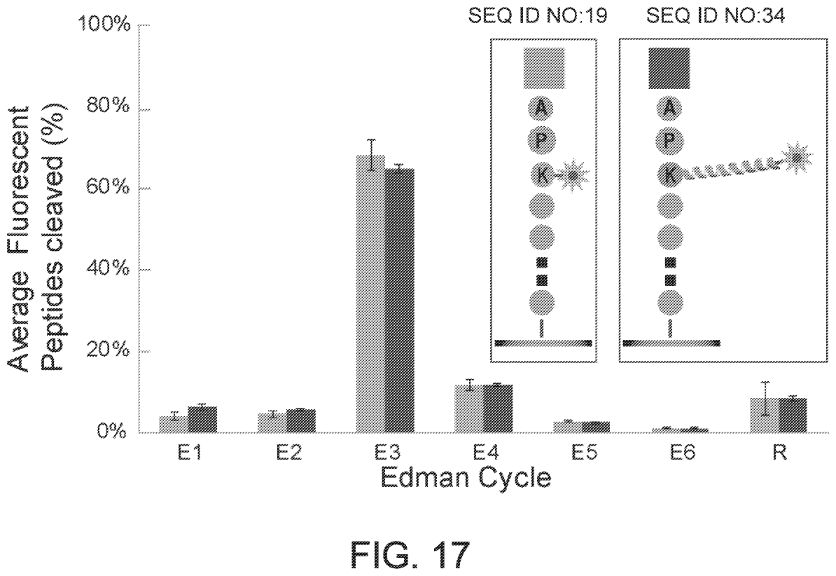

These polymers allowed the influence of quenching and FRET on peptides with multiple fluorophores to be investigated. To examine the occurrence of quenching, three Atto643 labeled polymers were installed on the peptide (JSP216), and through multiple fields of imaging, the intensity of peptides with one, two and three fluorophore signals could clearly be discriminated (FIG. 16B). The peaks' mean intensities of 17,472; 34,521; and 52,381 arbitrary fluorescence units closely match an additive model of a single fluorophore's intensity. Next, to evaluate a potential reduction in FRET, a peptide (JSP212) containing two distinct fluorophores, Janelia Fluor JF549 and Atto643, attached via polymers was synthesized. Less than 10% FRET efficiency and 67% colocalization of spots for a two fluorophore peptide (JSP212) between the donor, JF549 and acceptor Atto643, channels was observed (FIG. 16C). Labeling peptides with polymers did not impact the Edman efficiency (FIG. 17). These results demonstrate that, by substantially mitigating dye-dye interactions, polymers helped recover signal lost to FRET or quenching and consequently improve fluorosequencing data quality, as is further discussed in Example 6.

### Example 6

### Identifying Peptides in a Mixture Using an End-to-End Workflow

In this Example, the improved workflow and labeling chemistry was tested for its performance at identifying peptides in a mixture. The fluorosequencing of four individual synthetic peptides with similar sequences was fully characterized, with each peptide having 2 or 3 amino acids labeled with either Atto647N or Alexa555 polymers (or “promers”) (Table 6). As a guide to their subsequent interpretation, key experimental parameters from these and other control experiments were also determined, including Edman efficiency, dud-dye rates, and peptide detachment rates, in addition to those rates in Table 5. These rates are a key element of computational models for interpreting the fluorosequencing datasets, as is discussed below.

TABLE 6
PEPTIDES USED IN FOUR MIXTURE STUDY

| | | | ATTO643- | | ALEXA555- | |
|---|---|---|---|---|---|---|
| | | | PROMER | | PROMER | |
| PEP ID | PEPTIDE SEQUENCE | FLUOROPHORES | COUNT | POSITIONS | COUNT | POSITIONS |
| JSP234 | NH2- | F1 = DBCO-P30-ATTO643 | 2 | (2, 5) | 0 | − |
| | G{AZK}[F1]AG{AZK}[F1]AGAY | | | | | |
| | {PRA}R-CONH2 (SEQ ID | | | | | |
| | NO: 9) | | | | | |
| JSP251 | FMOC- | F1 = NHS-PEG4-N3, DBCO- | 1 | (2, 12) | 1 | (6) |
| | GK[F1]GAS{AZK}[F2]GANSAK | P30-ATTO643; F2 = DBCO- | | | | |
| | [F1]GSYRN{PRA}R-CONH2 | P30-ALEXA555 | | | | |
| | (SEQ ID NO: 10) | | | | | |
| JSP249 | FMOC-GK[F1]{AZK}[F2] | F1 = DBCO-PRO30- | 1 | (3) | 1 | (2) |
| | AGAY{PRA}R-COOH (SEQ ID | ALEXA555, NHS-PEG4-N3; | | | | |
| | NO: 11) | F2 = DBCO-PRO30- | | | | |
| | | ATTO643 | | | | |
| JSP252 | FMOC- | F1 = DBCO-PRO30-ATTO643, | 1 | (2) | 1 | (5) |
| | GK[F1]AG{AZK}[F2] | NHS-PEG4-N3; F2 = DBCO- | | | | |
| | AGSY{PRA}R-CONH2 (SEQ ID | PRO30-ALEXA555 | | | | |
| | NO: 12) | | | | | |

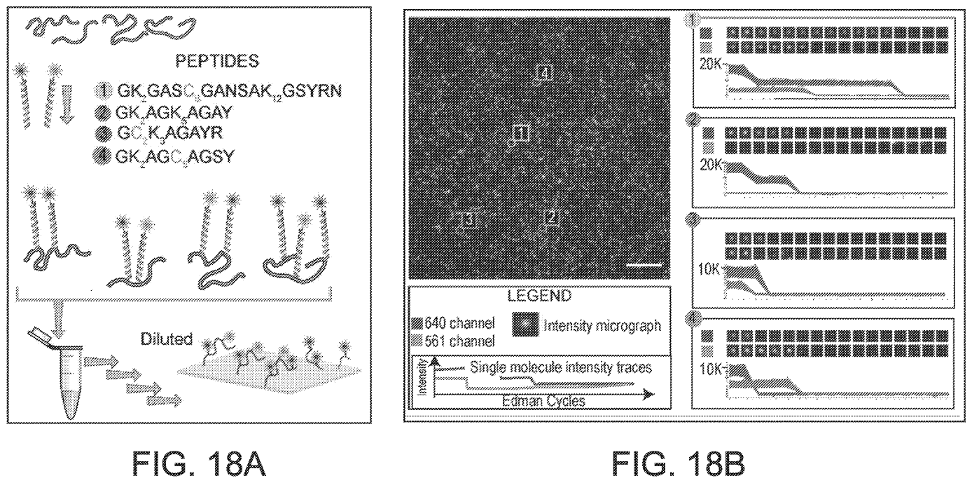

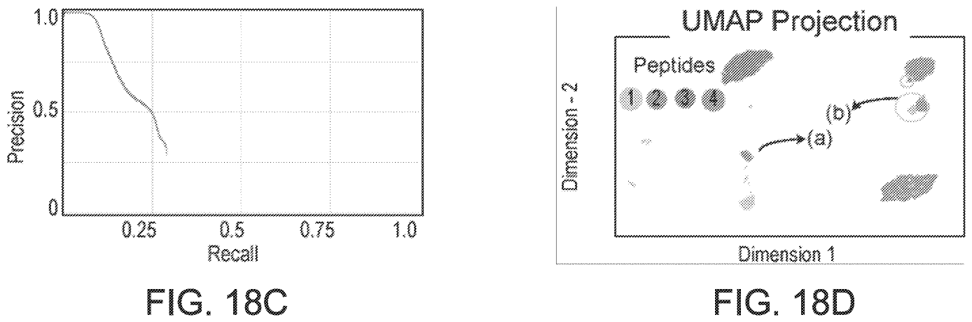

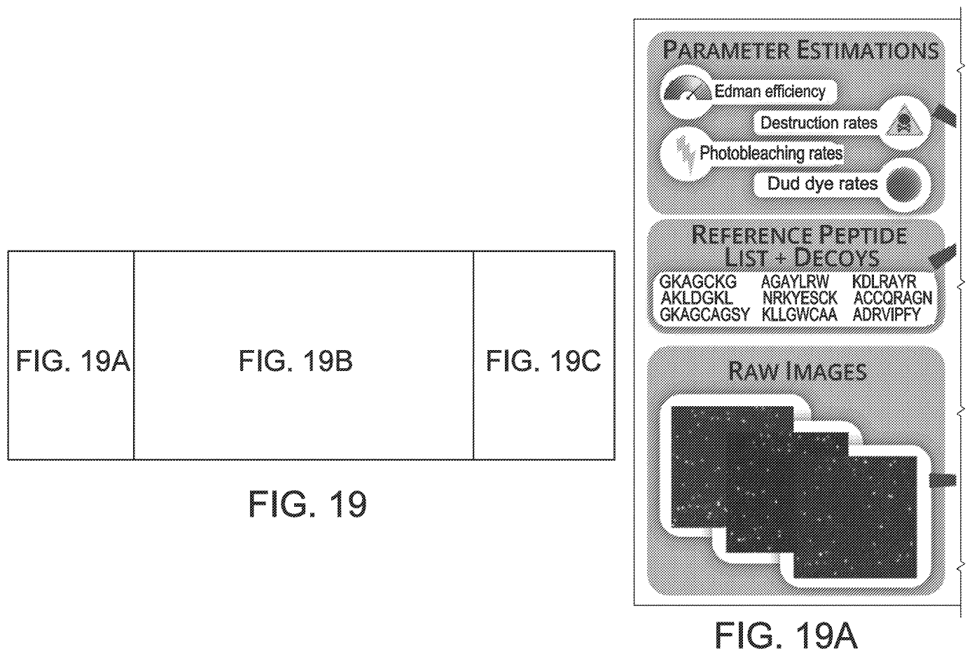

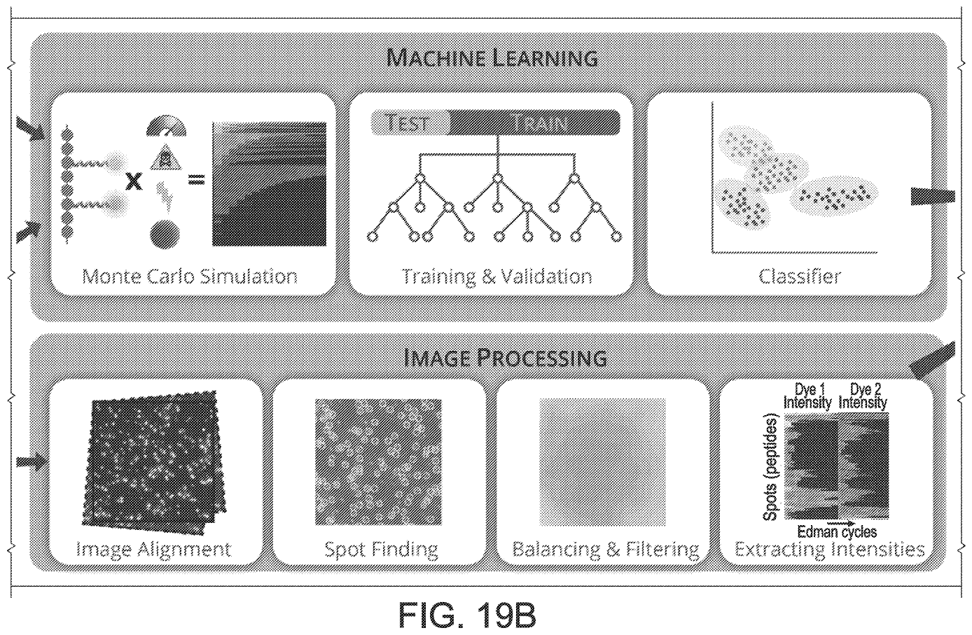

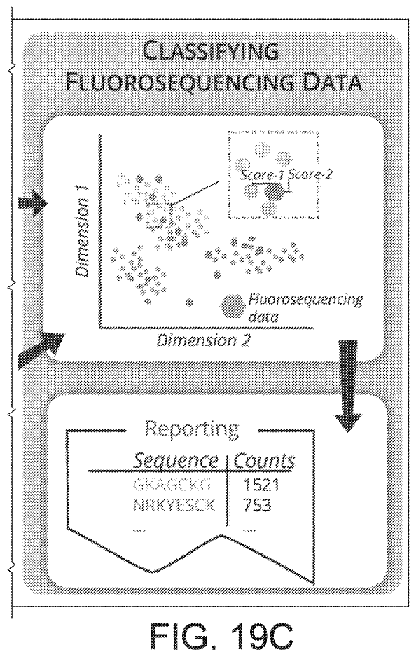

The peptides as a mixture were then considered: the four synthetic peptides were combined in an approximate equimolar ratio and 15 rounds of fluorosequencing was performed, collecting 100 fields of images across the two fluorescent channels (FIG. 18A). The left panel of FIG. 18B shows a representative image of the 4 peptide mixture, overlaid with two fluorescent channels and indicates positions of four distinct peptide molecules (labeled 1-4). The right panel shows expanded images of individual fluorescent peaks for the four peptides across the two fluorescent channels after each cycle of Edman chemistry, where stepwise reductions in fluorescent intensity can be readily observed. The images were then processed and the reads extracted and filtered (see Example 8, Materials and Methods) using a novel computational workflow (illustrated in FIGS. 19A-19C). This signal processing workflow involves analyzing TIRF microscope images to identify peaks and calculate each peak's intensity at the end of every cycle, thus generating an intensity track from all fluorescent channels (raw sequencing reads).

Peptide inference (in particular, peptide-read matching) was then performed using a machine learning classifier, trained to assign reads to peptides from the reference database based on simulating fluorosequencing with the same experimental parameters (efficiency of Edman cleavage, photobleaching rate, dud-dye rates, chemical destruction rates, etc). This assigns each read to a reference database peptide with an associated confidence score. A detailed description along with pseudocode is provided in Example 8, Materials and Methods. For the case of the four peptide mixture, a reference peptide database of 54 peptides was considered-containing the four synthetic peptides to be sequenced and an additional 50 decoy peptides, a randomly chosen set of 20-amino-acid long peptides. For computational modeling and inference purposes, the azido-lysine on the input peptide sequence was changed to cysteine. These simulated data were used as the training set for a random forest classifier (see Example 8, Materials and Methods) for identifying the real peptides from the larger reference database. More scalable approaches have also been developed to solve this problem (see, for example, Smith, Simpson, and Marcotte 2023 PLOS Computational Biology 19 (5): e1011157).

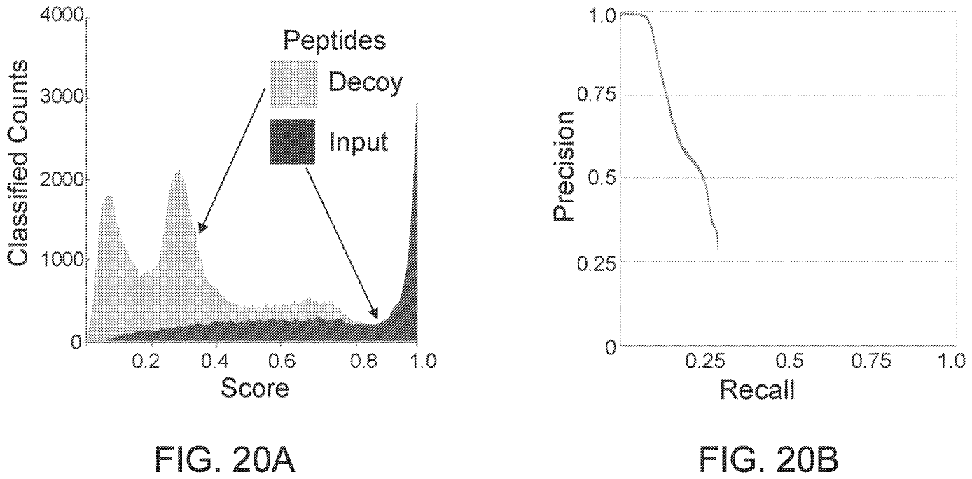

Using the machine learning classifier, each fluorosequencing read was scored to the most likely source peptide in the reference database. As plotted in FIG. 20B, scores above 0.99 predominantly identified the true peptides (see also Table 8). Plotting the corresponding Precision-Recall curve indicates that 6% of the raw reads can be correctly classified with 99% precision to the four input peptides (FIG. 18C). Visualization of the high scoring reads in a UMAP plot (McInnes, Healy, and Melville 2020 arXiv http://arxiv.org/abs/1802.03426) (FIG. 18D) shows clearly delineated clusters of fluorosequencing reads dominated by concordantly assigned reads, including separate clusters of the same peptide arising from distinct error modes, such as missing one Edman cycle (indicated by arrow (a)) or observation of a dud dye (indicated by arrow (b)), that can nonetheless still be correctly mapped to their corresponding peptide sequences.

Overall, these data demonstrate improvements to the fluorosequencing workflow by sequencing a mixture containing four synthetic peptides. After training a machine learning classifier on these peptides and 50 randomly generated decoy peptides, 6% of the fluorosequencing reads with 99% precision were able to be correctly classified, providing general support that the improvements to peptide labeling and workflow enables accurate peptide sequence determination.

### Example 7

### Targeted Sequencing of an Antigenic Peptide

In this Example, the ability to accurately identify a target antigenic peptide through a multi-omic study was tested. A clinical application was assessed by determining whether the enhanced workflow could detect HLA-I peptides from a small reference database of competing HLA-I peptides. The identification of tumor-associated peptide antigens in limited clinical biopsies poses a considerable obstacle for existing technologies, consequently hindering the advancement of novel therapies in immuno-oncology (Schumacher and Schreiber 2015 Science 348 (6230): 69-74; Vizcaíno et al. 2020 Molecular & Cellular Proteomics: MCP 19 (1): 31-49).

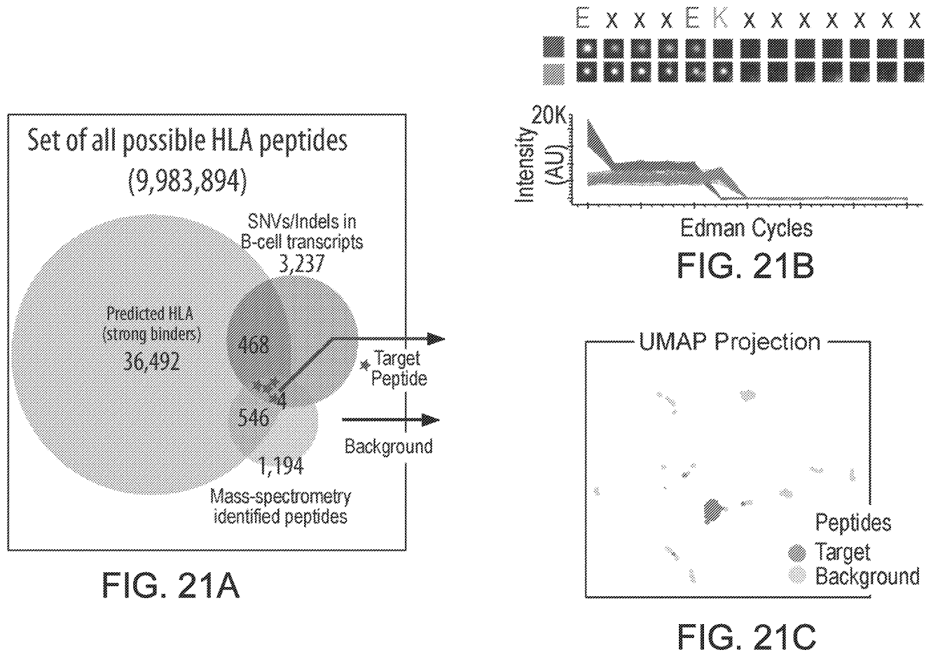

A pilot-study was set up where ˜300 million mono-allelic B-cells (HLA A2603) was cultured and the observed and potential HLA-I peptide repertoire through three orthogonal techniques was characterized (FIG. 21A). Despite the observed genomic variants and 3,237 predicted strong binding neoantigenic HLA peptides found through RNA sequencing, the vast majority of HLA-I peptides (1,189) identified by MS were distinct HLA-I peptides. Overall, fewer than 50% of the MS-observed peptides were predicted computationally to be strong HLA binders, possibly due to bias in current computational prediction algorithms (“The Problem with Neoantigen Prediction” 2017 Nature Biotechnology 35 (2): 97-97). Four putative peptides were observed, identified through mass spectrometry and containing an alternative residue relative to the Hg38 reference human genome, serving as model neoantigens. One such peptide was synthesized, fluorescently labeled, and sequenced (JSP308, Table 3). Fluorescent radiometries indicated the correct step drop position for the Atto643 and Texas-red labeled residues (FIG. 21B). As above, fluorosequencing of a reference set of 37 HLA-I peptides (the set of MS-identified HLA-I peptides with more than two peptide spectral counts) was simulated including the input peptide, and machine learning classifier was trained to assign reads to the reference database peptides. Each experimental read was then assigned to a reference database peptide using this classifier, measuring classification accuracy and visualizing the reads by UMAP (FIG. 21C). As with the four peptide mixture, the target HLA-I peptide could in general be clearly distinguished from the background set. This trend is expected to continue to improve with additional dye colors (allowing more residue types to be labeled) and other improvements, suggesting fluorosequencing may be applicable to the challenge of targeted identification of low abundance HLA-I peptides.

Overall, this Example demonstrates a pilot experiment to evaluate the ability to accurately identify a target antigenic peptide through a multi-omic study on an HLA A2603-expressing monoallelic B cell line. Using HLA prediction software, the set of expressed HLA peptides was found using genomic sequence and SNV comprising transcripts and compared with those HLA-I peptides identified experimentally from the cell-line using tandem mass-spectrometry. Four peptides were identified as a set of putative neoantigens. Fluorosequencing of one of HLA-I peptides against a reference background illustrates the potential and sensitivity for targeted clinical assays.

In addition, computationally inferring peptide identity from fluorosequencing data has also suggested a workflow for virtual fluorosequencing, for example, in silico modeling the entire sequencing process from proteins to expected fluorescence patterns for each possible generated peptide. The model is based on a rigorous characterization of the physicochemical processes and estimation of parameters, such as the probability of Edman failure, peptide detachment rate, chemical destruction rate, and the like (M. B. Smith et al. 2023). Virtual fluorosequencing may be used to guide experimental design by suggesting the protease and amino acids to label to yield the fluorescent pattern that best identifies the peptide or protein of interest, as well as to identify the most sensitive parameters that, when improved, would lead to better peptide identifications. By guiding experimental design, virtual fluorosequencing may provide a valuable tool for improving the reliability of fluorosequencing.

The successful completion of the end-to-end process for fluorosequencing of peptide mixtures is a major milestone for the technology. In this study, several of its key features were demonstrated, including high sensitivity and the generation of rich quantitative data, that make fluorosequencing a promising tool for clinical applications needing accurate peptide and protein measurements from low sample amounts.

### Example 8

### Materials and Methods

In this Example, materials and methods are described for the experiments in Examples 1-7.

### Purification Methods

Using analytical column chromatography: An HPLC system (1100C, Agilent) was used with an analytical column (Kinetex 5 μm XB-C18, 250×4.6 mm, 100 Å) operating at a flow rate of 1 mL/min and a 30 min gradient (5-95% Acetonitrile/water with 0.1% Formic acid) to purify samples with <1 mg peptide or fluorescently labeled peptide. The different fractions were collected using the attached fraction collector and the species were analyzed using LCMS. The purified peptide fraction was then dried down using speed-vac (Eppendorf) and solubilized in 50% Acetonitrile/water for further characterization.

Using preparative or semi-preparative scale HPLC: A semi-preparative or preparative HPLC system was used for samples containing larger amounts of peptides (>1 mg). The preparative scale purification involved using an Agilent Zorbax column (4.6×250 mm) on an HPLC system (Shimadzu model) operating at a flow rate of 10 mL/min, and eluting with a 90-minute gradient of 5-95% acetonitrile (0.1% Formic acid). For the semi-preparative scale purification, the product was purified using HPLC (Shimadzu) with a semi-prep column (Hichrom C8, 5 micron, 10 cm×10 mm, 150 Å) operating at a 5 mL/min flow rate and an elution gradient of 5-95% Acetonitrile (0.1% Formic acid) over 60 minutes. The fractions were analyzed using mass spectrometry and pooled the fractions containing the product. Their volume was then reduced using a roto-vap (IKA, RV10) and lyophilized the samples (VirTis SP Scientific, BTP-8ZLOOW) prior to characterization.

SDS PAGE gel purification: For peptides labeled with fluorescent polymers, standard SDS PAGE electrophoresis was performed on a 16.5% Tris/Tricine SDS PAGE gel, using vendor detailed protocol (Biorad, Cat #1610739, #4563065, #1610744). After washing the gel and confirming the fluorescent bands using a gel imaging station (Amersham Imager 600 gel dock), the bands of interest were cut using a razor blade. The excised pieces were crushed and submerged in 50% vv of Acetonitrile/water in a microcentrifuge tube. They were then sonicated for 5 minutes and heated them at 60 C for 30 minutes to extract the peptides from the gel.

### Synthesis of Peptides

All peptides were either custom synthesized from Genscript (NJ, USA) or synthesized in-house using a standard automated solid-phase peptide synthesizer (Liberty Blue microwave peptide synthesizer; CEM Corporation). If synthesized in-house, the peptides were cleaved from beads with standard TFA cleavage cocktail (comprising 95% TFA, 2.5% water and 2.5% Triisopropylsilane (Sigma, Cat #233781) for 2-4 hours at room temperature, followed by ether precipitation. After that, the crude precipitate was purified by preparative scale HPLC. The synthesized peptides were characterized by LCMS.

### Synthesis of Polymers (or “Promers”)

The amino acid sequence of certain example polymers, as read from N-terminus to C-terminus, is Fmoc-G-PEG₂-G-P₃₀-K(boc)-CONH₂ (boc=tert-butyloxycarbonyl protecting group) (SEQ ID NO:35). Standard solid-phase Fmoc synthesis was used, and double coupling of proline residues was performed after the first 20 amino acid synthesis to build the rest of the chain. The terminal Fmoc group was removed and reacted the resin with 5 equivalents (eq) of DBCO-NHS (dibenzocyclooctyne-N-hydroxy succinimidyl ester) in dry DMF, containing 2.0 eq of triethylamine for 2 hours at 37° C. to functionalize the polypeptide with DBCO. After washing and cleavage and HPLC purification of the DBCO-functionalized polypeptide, the ε-lysine was labelled on the polypeptide with 1.2 eq of succinimidyl ester derivatized fluorophores (Atto643, JF549, or Alexa555) by incubating it in 1:1 HEPES (0.1M, pH 8.5), acetonitrile buffer for 2 h at 37° C. The fluorescent polymer-DBCO product was HPLC purified using a semi-preparative column.

### Fluorescent Labeling of Peptides

Direct fluorescent labeling: In general, 0.9 eq of NHS functionalized dye was coupled with ˜100 nmoles of peptides, per lysine, by incubating the mixture in 100 μL of 1:1 vv Acetonitrile/HEPES buffer (pH 8.5, 0.1M) at room temperature for 2 hours. After the reaction, the peptide was purified using an analytical column. For peptides containing both lysine and cysteine residues (Peptides: JSP129, JSP150 and JSP157) and needing two different fluorophores, the cysteine residue was first labeled by incubating the peptide with 1.2 eq of Atto647N-Iodoacetamide (Atto-tec, Cat #AD 647N-111) in 100 μL of sodium phosphate buffer (pH 7.5; 0.1M) and incubating for 2 hours at room temperature. The pH was then adjusted to 8.5 by adding 50 μL of 1M HEPES (pH 8.5) and added 1.2 eq of Fluorophore-NHS. This was incubated overnight at room temperature, followed by HPLC purification using the analytical column.

Labeling with fluorescent polymers: Across different peptides, 100 nmoles of peptides containing azidolysine was incubated directly with 2 eq of DBCO functionalized dye polymers in 100 μL of 1:1 vv Acetonitrile/HEPES buffer (pH 8.5, 0.1M) at room temperature for 16 h. After the reaction, the peptide was purified using the analytical HPLC. If peptides need two different fluorophores, a two-step method was used by first labeling the azidolysine residue with the polymer, followed by functionalizing the lysine residue with NHS-PEG4-Azide (Broadpharm, Cat #BP-20518). The resulting peptide was purified using the analytical column (described earlier) and labeled the second fluorophore using the same DBCO azide chemistry. Finally, the two-color peptides were purified using either the analytical HPLC column or SDS PAGE gel.

### Isolating and Identifying HLA Peptides Using Tandem Mass-Spectrometry

The monoallelic B-cell line (B721.221), with HLA allele A*2603, was purchased directly from the International Histocompatibility Working group (Seattle, Washington). The cells were cultured and HLA peptides were isolated from approximately 300 million cells as previously described (Abelin et al. 2017). The isolated HLA peptides were identified using an LC-coupled tandem mass-spectrometer (ThermoFisher, Orbitrap Fusion Lumos) and a reference dataset of a human proteome (Swissprot), applying settings from the literature for analyzing HLA peptides (Bassani-Sternberg et al. 2015 Molecular & Cellular Proteomics: MCP 14 (3): 658-73) and ProteomeDiscoverer 2.3 software (Thermofisher).

Predicting HLA Antigenic Peptide from Genomic and Transcriptomic Data

The RNA sequencing data for the B cell-line (expressing HLA-A2603 allele) was obtained from a publicly available dataset (Abelin et al. 2017 Immunity 46 (2): 315-26). RNAseq alignment was preformed using STAR tool (Dobin et al. 2013 Bioinformatics 29 (1): 15-21) and single nucleotide polymorphism analysis by comparing the aligned transcriptome to the standard human genome (Genome Reference Consortium Human Build 38) using GATK best practice pipeline (https://gatk.broadinstitute.org/hc/en-us). 50 base pairs of 5′ and 3′ were manually translated from the identified functional single nucleotide variant (Wang, Li, and Hakonarson 2010 Nucleic Acids Research 38 (16): e164) to peptide sequences. The presented HLA peptides were then predicted using netMHCPan 3.0 software (Lundegaard et al. 2008 Nucleic Acids Research 36 (Web Server issue): W509-12). The peptides were subsequently filtered for strong binding to the HLA allele A*2603.

### Characterization of Peptides

LC/MS: The peptide samples were characterized using Liquid chromatography mass spectrometry (LCMS) with an Agilent 1260 system equipped with Single Quadrupole LC/MS (6120B) and a 5 μm C18 column (Agilent Zorbax Eclipse Plus, PN 959746-902). The samples were injected and subjected to an elution gradient of 5-95% aqueous (0.1% FA) over 12 minutes with a co-eluent of Acetonitrile. Agilent Chemstation (ver #Rev C.01.10 [287]) was used to analyze the data.

MALDI: Peptides containing polymers were characterized with a mass range of 3K-20K using MALDI TOF (Autoflex max, Bruker). 1 μL of solubilized sample was spotted onto a clean MALDI Target plate (MTP 384 target plate polished steel, Bruker) and mixed with 1-2 μL of 40 mg/mL DHB (Thermofisher, Cat #90033) in 70% Acetonitrile and 0.1% Trifluoroacetic acid. After drying, the sample was analyzed in reflective mode at a laser power of 60-90%. Autoflex analysis software (ver #3.4) was used to analyze the data.

### Fluorescence Reading Using Plate Reader

Fluorescent peptides were diluted into methanol to an ˜10 μM concentration and fluorescence measured using the fluorescence plate reader (Synergy H1 microplate reader, Biotek-Agilent). The samples are excited at 500 nm and emission measured from 520-700 nm in increments of 10 nm. No gain setting was used.

### Silane Functionalization of Glass Slides

40 mm glass cover slides (Bioptechs, Cat 40-13 13-03 192) were cleaned for 10 minutes on each side using an UVO cleaner (Jelight, Model 18). After cleaning, the slides were placed vertically in a Teflon slide rack (custom-made). 100 μL of 3-azidopropyltriethoxysilane (Gelest, SIA0777, CAS #83315-69-9) was then pipetted into the lid of a Teflon Reaction Vessel (Alpha Nanotech Inc) and placed both the slide rack and the cap in a Pyrex desiccator chamber, which was preheated to 80 C. The valve of the desiccator was attached to a vacuum pump and a vacuum was drawn until the pump stabilized at approximately 0.08 MPa. The desiccator was then placed in an 80 C oven and allowed to sit for 16 hours. The silane-functionalized slides were stored in vacuum-sealed bags at 4 C until use.

### Peptide Immobilization

Peptides (containing alkyne) were covalently coupled to the coverslip surface via copper-catalyzed click chemistry between the alkyne-modified C-terminal AA residue and the azido silane. A fresh solution of 2 mM copper sulfate, 1 mM tris(3-hydroxypropyltriazolylmethyl)amine (Sigma, Cat #762342), 20 mM HEPES (pH 8.0), and 5 mM sodium ascorbate with fluorescently labeled angiotensin was incubated for 30 min at room temperature on the coverslip, washed with water to remove unbound peptides, and dried under a nitrogen gas stream.

### Total Internal Reflection Fluorescence (TIRF) Microscopy

Two similar Nikon Ti microscopes, equipped with a CFI Apo 60×/1.49NA oil-immersion objective lens and a 1.5× tube lens, a motorized stage, a sCMOS camera, and a laser excitation were used for all the experiments. Details of these parts and the fluorescent channel configurations are provided in Example 9.

### Automated Fluidics for Performing Edman Sequencing Chemistry

Fluidic setup: The pumping of different solvents was automated using a syringe pump (Tecan Cavro, Model #20738291) (3 way valve configuration) and a 10-port multi-position valve system (Valco Instruments, Model #EUHB), as described in the earlier publication (Swaminathan et al. (2018) Nat. Biotechnol. 36, 1076-1082). The sample temperature was maintained at 40° C. (for System A) and 50° C. (for System B) by heating both the perfusion chamber and microscope objective for Edman sequencing experiments. Solvent exchanges were controlled in the fluidic device using in-house Python scripts and coordinate with image acquisition via custom macros in the Nikon Elements software package. The reagents/solvents were connected to the different valves and detail the steps for performing Edman chemistry in as in Table 7.

TABLE 7
DESCRIPTIONS OF THE SOLVENTS CONNECTED
TO THE 10 PORT VALVE

| Valve | | |
|---|---|---|
| Number | Name | Reagents |
| 1 | Waste | — |
| 2 | Imaging solvent: | 1 mM Trolox added to 5 mM HEPES in Methanol. Solution |
| | | is degassed |
| 3 | Base Solution: | Pyridine + N-Methylmorpholine (38:1 Pyr:NMM); Final |
| | | concentration of NMM is 60 mM |
| 4 | PITC | System A: Phenyl isothiocyanate solution |
| | | System B: Phenyl isothiocyanate + hexafluoroisopropanol |
| | | (19:1 vv PITC:HFIP) |
| 5 | Wash | 20% piperidine in Acetonitrile |
| 6 | Edman reagent | — |
| | mixing chamber | |
| 7 | Water | water |
| 8 | TFA | Trifluoroacetic (neat) |
| 9 | — | — |
| 10 | Flow cell | — |
| | Backing solvent | Acetonitrile |

### Signal Processing

Raw sequencing reads were extracted from the time series micrographs as detailed in Example 9 and implemented in the Python package sigproc_v2, available from https://github.com/marcottelab/robust-fluorosequencing-plaster. Briefly, images capturing the same field of view were corrected for variation in signal intensity by regional illumination balancing and bandpass filtering of both background and over-saturated pixels, aligned across cycles to account for stage movement, and peaks identified by convolving with the point spread function (PSF). From radiometry on each peak, intensity parameters were calculated for each peak across Edman cycles to create a raw fluorosequencing read for each candidate peptide. Fields were filtered on the basis of image anomalies or poor alignments, and individual reads were rejected based on their fit to the PSF, with poor fits suggesting the presence of more than one molecule. Additional filters are described in Example 9.

### Scoring Co-Localization

For each raw fluorosequencing read (acquired, extracted, and filtered as described above) the intensities were normalized to the mean one-count intensity (μ) and plotted by channel pairs as scatter plots; FIG. 16D shows an example. Signal above the dark thresholds (determined at 3σ above the background mean) in both channels were considered colocalized and were calculated as the percentage of the total reads plotted.

### Measuring FRET Efficiency

For FRET analysis, the donor (lower wavelength) and acceptor (higher wavelength) dyes were imaged as separate channels using their respective laser and filters. A “FRET channel” was additionally defined using the donor's excitation laser and filter and the acceptor's emission filter. Details of the System A setup can be found in Example 9. Data was acquired in all channels and the reads were calculated and filtered as described above. These reads were then used to calculate the FRET efficiency as in (Hellenkamp et al. 2018 Nature Methods 15 (9): 669-76; Zal and Gascoigne 2004 Biophysical Journal 86 (6): 3923-39). To simplify the analysis, peptides were also filtered for with signals in all channels above the contamination background.

FRET efficiency (E) for each read was calculated using Eq. 1, where I_{F}, I_{D}, and I_{A} represent the fluorescence intensities of the FRET, donor, and acceptor channel reads, respectively.

$$E = \frac{( {I}_{F} - {α I}_{D} - {δ I}_{A} )}{( {I}_{F} - {α I}_{D} - {δ I}_{A} ) + γ {I}_{D}} \qquad ( 1 )$$

This formula requires the determination of experimental coefficients to account for the modifications of the intensities inherent in this type of data acquisition. The first of these coefficients (α and δ) account for signal leakage into the channels that occurs from overlapping excitation or emission (crosstalk). To calculate these, reads from a control experiment with single labeled peptides were plotted in the same manner as the colocalization analysis. The slope of the correlation between the signals in each channel pair was used as the crosstalk coefficient giving a value of 0.20 for the donor to FRET channel (α) and 0.03 for the acceptor to FRET channel (δ) for the peptides in FIG. 15B. The last coefficients (γ and β) is to normalize effective fluorescence quantum yields, as these experiments are on purified peptides, the ‘single-species’ method described in Hellenkamp et al. were used giving a value of for γ and β as 1.2 and 2.9 for the peptides shown. To confirm the these coefficients the stoichiometry (S) was calculated for each peak using Eq. 2, and confirmed that the mean stoichiometry was the expected 0.5, for these peptides having one donor and one acceptor fluorophore.

$$S = \frac{( {I}_{F} - {α I}_{D} - {δ I}_{A} ) + γ {I}_{D}}{( {I}_{F} - {α I}_{D} - {δ I}_{A} ) + γ {I}_{D} + \frac{{I}_{A}}{β}} \qquad ( 2 )$$

### Machine Learning Classifier

To generate synthetic training and testing data typical of fluorosequencing experiments, Monte Carlo simulations were performed as in Smith, Simpson, and Marcotte 2022 PLOS Computational Biology 19 (5): e1011157, explicitly modeling the Edman failure rate, peptide detachment rate, and N-terminal blocking rate, as well as a number of fluorophore-specific parameters, including each dye's average fluorescence intensity, standard deviation of intensity, standard deviation of background intensity, missing (“dud”) dye rate, and dye destruction rate (a combination of chemical destruction and photobleaching rates, the latter of which is small in the conditions used for imaging). Methods for estimating these parameter values are detailed in the Example 9 (with additional methods available in Smith et al. 2023 PLOS Computational Biology 19 (5): e1011157 and all parameters are provided in Table 5. These synthetic reads were expressed as double-precision floating point vectors comprising (simulated) fluorescence intensities for each Edman cycle and fluorescent channel. By considering these reads as feature vectors, a random forest classifier was trained to assign reads to source peptides, as implemented with scikit-learn and default settings.

### Example 9

### Additional Materials and Methods

In this Example, additional materials and methods are described for the experiments in Examples 1-7.

### SDS Page Gel Purification

Peptides labeled with fluorescent polymers were mixed with a Tricine Sample Buffer (BioRad, Cat #1610739) and loaded them onto a 16.5% Tris/Tricine SDS PAGE gels (BioRad, Cat #4563065) with Tris/Tricine running buffer (BioRad, Cat #1610744) while excluding traditional reduction and heating during sample preparation. Gel electrophoresis (Biorad, Cat #4006213) was performed on the loaded sample, until loading dye ran off the gel. After washing the gel, the gels were imaged using a gel imaging station (Amersham Imager 600 gel dock) in the 530 and 630 nm fluorescent channels. Bands of interest were cut out using a razor blade, washed the excised pieces with water, and then crushed and submerged them in a 50% vv of Acetonitrile/water in a microcentrifuge tube. They were sonicated for 5 minutes and heated them at 60° C. for 30 minutes to extract the peptides from the gel. Then, the supernatant was removed and a C18 ziptip used (Thermofisher, Cat #) to desalt and purify the peptides. The excised peptides were characterized using LC-MS or MALDI. Polymer labeled peptides were found to have a different migration speed than protein standards; a 10 kDa peptide with polymers had a similar retention time as a 25 kDa Protein standard (Precision Plus Protein Dual Xtra Standard, Cat #1610377).

### Detailed Synthesis of Polymers (Promers)

An automated peptide synthesizer (Liberty Blue microwave peptide synthesizer, CEM Corporation) was used to synthesize the polymer backbone of the polymers. Fresh stock solutions of each amino acid building block were prepared (Fmoc-glycine, Fmoc-proline, Fmoc-lysine (boc), and Fmoc-PEG2) at a concentration of 0.2M, as well as coupling reagents (1M of Oxyma base and 1M of DIC). After coupling the first 20 monomers on the resin, double coupling of Fmoc-glycine, Fmoc-proline, and Fmoc-Peg2 was performed. Then, the terminal Fmoc group was removed and the resin was reacted with eq of DBCO-NHS in dry DMF, containing 2.0 eq of Triethylamine for 2 hours at 37 C to functionalize the polypeptide with DBCO. Next, the DBCO-functionalized polypeptide was washed and cleaved from the resin with an acidic cocktail consisting of 50% TFA, 45% DCM, 2.5% Triisopropylsilane, and 2.5% water for 2 h at room temperature. The cleavage cocktail was dried via N2 gas until ≤5% of the initial volume remained, then 10:1 vv of cold ether was added to precipitate the peptides. After decanting off the ether, the resulting solid product was re-solubilized in 50% Acetonitrile/Water for purification. The DBCO-polypeptide was purified using HPLC with a semi-prep column (Hichrom C8, 5 micron, 10 cm×10 mm, 150 Å) operating at a 5 mL/min flow rate and an elution gradient of 5-95% Acetonitrile (0.1% Formic acid) over 60 minutes. Then, the labeled peptides were purified using HPLC and the same semi-prep column as described earlier.

Synthesis of Peptides with N-Terminal Branched Proline Polymer

A peptide with the sequence Boc-Lys[fmoc]-Gly-azLys-Gly-Pra-Gly-Resin (SEQ I_{D} NO: 32) was synthesized on Tentagel Rink Amide Resin by using boc-lysine (fmoc) to enable the synthesis of variable length proline backbones from the lysine side chain (azLys denotes azido-lysine; Pra denotes Propargylglycine). After synthesizing the proline polymer, a terminal glycine residue was installed. Then, the N-termini was labeled on the branched glycine with either TMR-NHS or JF549-NHS dye (1.2 eq) on the resin by incubating it in DMF and 2 eq of Triethylamine for 2 h at room temperature. After cleaving the peptide and purifying it using preparative scale HPLC, the azidolysine on the peptide was labeled with 1.2 eq of DBCO-Peg4-Atto647N (custom synthesized by Atto-tec) by incubating the mixture overnight at room temperature.

### Fluorophore Selection Through Solvent Stability Screen.

The fluorophores were obtained commercially or obtained through collaborators. 70 fluorophores were screened to identify those most resistant to the Edman solvents by covalently attaching the dyes to Tentagel beads (Chem-Impex International, 04773) and their fluorescence measured after a 24-h incubation with TFA, pyridine/PITC (9:1 vv), Methanol and Piperidine at 40° C. Nonspecifically bound fluorophores were removed by repeated washing with dimethylformamide (DMF), dichloromethane, and methanol. These beads labeled with fluorescent dyes were suspended in 100 μL of phosphate-buffered saline (PBS, pH 7.2) in a 96 well plate. The fluorescent bead images were captured across multiple channels, using an Epi-microscope and calculated the change in fluorescent intensity, compared to the methanol control. Custom script was used to measure the bead fluorescence from the images.

Epi-microscope (Nikon Eclipse TE2000-E inverted microscope) used was equipped with an Apo 60×/NA 0.95 objective, Cascade II 512 camera (Photometrics), a Lambda LS Xenon light source and a Lambda 10-3 filter-wheel control (Sutter Instrument), and a motorized stage (Prior Scientific), all operated via Nikon NIS Elements Imaging Software. Images were acquired at one frame per second through a 89000ET filter set (Chroma Technology) with channels ‘DAPI’ (excitation 350/50, emission 455/50), ‘FITC’ (excitation 490/20, emission 525/36) ‘TRITC’ (excitation 555/25, emission 605/52), and ‘Cy5’ (excitation 645/30 emission 705/72).

### Total Internal Reflection Fluorescence (TIRF) Microscopy

Single-molecule TIRF microscopy experiments were performed on two different Nikon systems, detailed below:

### System A

A Nikon Ti-E inverted microscope was used equipped with a CFI Apo 60×/1.49NA oil-immersion objective lens and a 1.5× tube lens, a motorized stage (TI2-S-HW, Nikon Inc Scientific), an 1022×1022 pixel sCMOS detector (pco.edge, PCO), and a LUNF-XL (Nikon) laser including 561 and 647 nm lasers and filter cube containing 405/488/561/638 quad dichroic and barrier filters, an emission filter wheel with band pass filters detailed below (all filters, Chroma). Each image represents a 72 μm×72 μm square region of the sample. The different channels are considered as a combination of incident laser wavelength and the corresponding bandpass filter. The “561 channel” includes excitation with the 561 nm laser (9.5 mW, 50%) through quad dichroic and emitted signal is collected through emission filter EM-603/30. The “640 channel” includes excitation with the 640 nm laser (2.5 mW, 10%) and emitted signal is collected through quad dichroic and EM-705/72 emission filters. The “FRET channel” includes excitation with the 561 nm laser (9.5 mW, 50%) through quad dichroic and emitted signal is collected through emission filter EM-705/72. Laser powers were measured after the objective.

### System B

A Nikon Ti-E inverted microscope was used equipped with a CFI Apo 60×/1.49NA oil-immersion objective lens and a 1.5× tube lens, a motorized stage (ProScan II, Prior Scientific), a scientific CMOS camera equipped with a 2048×2048 pixels (binned to 1024×1024 pixels) (Hamamatsu, Model #C15440) and a MLC400B (Keysight) laser including 561 and 640 nm lasers and filter cube containing 405/488/561/638 quad dichroic and barrier filters, an emission filter wheel with band pass filters detailed below (all filters, Chroma). Each image represents a 72 μm×72 μm square region of the sample. The different channels are considered as a combination of incident laser wavelength and the corresponding bandpass filter. The “561 channel” includes excitation with the 561 nm laser (9.4 mW, 70%) through quad dichroic and the emitted signal is collected through emission filter EM-603/50. The “640 channel” includes excitation with the 640 nm laser (2.5 mW, 20%) and the emitted signal is collected through quad dichroic and EM-705/72 emission filters. Laser powers were measured after the objective.

### Calibration Imaging Experiments

Each system is calibrated regularly to determine channel offsets, illumination flatness, and regional point spread functions (PSF). For the channel offsets 100 nm Tetraspeck Fluorescent Microspheres (ThermoFisher, Cat #T7284) are diluted in 100 μL methanol solvent and spotted onto a glass slide to dry, adjusting the dilutions to achieve approximately 100 peaks per field. Images were captured in all channels over 100 fields. Each microsphere contains dyes spanning multiple fluorescent channels and can be used to determine any fixed lateral offset between channel images. For the other metrics either the tetraspec calibration data or experimental samples of single count peptides were used.

Images were then analyzed using the “calibration_sigproc” workflow, which, for multi-channel data, first performs a subpixel alignment via gradient descent after a fast Fourier transform to provide the per channel offsets. Next the peaks are identified in a similar manner as experimental data (detailed below in signal processing) however here each channel is treated independently. Using the calculated peak, the PSF is parameterized, which models the shape and intensity of a single fluorescent peptide, by fitting each peak to 2d gaussians. The images were split into 25 sub regions and then the average PSF was calculated using all peaks and all fields, given a regional expectation for the PSF. Lastly, from the individual fits, the location and intensity of each peak was calculated, which are used as a spatial indicator of illumination. By combining this information across all fields, a geometric expression of the regional illumination for each channel is obtained.

### Signal Processing

Signal processing includes the series of image processing steps converting multichannel images captured through the Nikon microscope (.nd2 files) after every Edman cycle into intensity arrays for each image channel across the cycles for every peptide spot. Glossary of terms used are shown in Table 2.

### Steps

1. Nd2 files are converted to npy files. After every Edman cycle, the images are saved in Nikon's proprietary nd2 file, which comprises images from multiple channels and fields. Using an n2 converter python package, the nd2 files (one per cycle, containing all channels for all fields) are converted to numpy array files (one per field, containing all cycles for all channels). During this conversion process, a per-channel/cycle field quality metric is computed using a low-pass Fast Fourier Transform (FFT) filter to measure low-frequency power in the image. This per-channel/cycle metric is averaged to arrive at a field-quality measurement, which may be used for filtering ahead of classification.

```
Pseudo-code:
OUTPUT: numpy array (.npy file) per field, after reorganizing the contents of nd2 files,
one per cycle.
FOR every imaging cycle
  GET nd2 file that contains data for multiple channels and multiple fields
  OUTPUT .npy temporary files for each channel,cycle per field
  USING nd2 python library
FOR every field
  GET all channel/cycle information from temporary .npy files for this field
  OUTPUT single .npy file containing all cycles/channels for this field
  USING nd2 python library
  COMPUTE and save low-frequency-power as a measure of field quality
```

2. Regional illumination balancing is applied to account for variations in signal intensity over different regions within each image. Each image is balanced to overcome non-uniform signal intensity (e.g. “vignetting” inherent when using spherical optics) based on the regional illumination measured during calibration experiments described above.

```
Pseudo-code:
OUTPUT: regionally balanced image as numpy array.
FOR every field
  FOR every channel
    GET experimentally-determined balance_image for this channel
    FOR every cycle
      Divide channel-cycle image by balance_image
      SAVE regionally-balanced image
```

3. Band-pass signal filtering. Every regionally-balanced image is transformed into the frequency domain using a standard FFT algorithm (numpy) and reject signals above and below empirically-determined threshold frequencies to remove both background (reject-low) and over-saturated (reject-high) signals.

```
Pseudo-code:
OUTPUT: band-pass filtered image data
FOR every field
  FOR every channel
    FOR every cycle
      USING regionally-balanced image
      REMOVE signal above and below reject thresholds via FFT
filter
      SAVE filtered image
```

4. Subpixel image-alignment, shift, and resample. All images for a given field are aligned across cycles to account for stage movement between cycles. This involves, first an alignment done on one channel for all fields then the channel offsets, determined during calibration, is applied to the remaining channels. For the fixed channel alignment, a first-pass pixel-level alignment is performed via OpenCV's filter2d convolution, giving pixel-offsets for each image relative to the first cycle's image. Then, the sub-pixel offset is determined using a gradient-descent in Fourier space to achieve sub-pixel accuracy. An alignment score for each field is calculated as the maximum shift in pixels required to align all system cycles, which is used downstream to filter out images prior to analysis.

```
Pseudo-code:
OUTPUT: resampled aligned images + alignment scores for every field
stack
FOR each field
  FOR each channel used for alignment
    FOR every cycle in the experiment
      ALIGN the filtered images in the field stack to the first one
      SAVE the pixels-shifted pair value per image (alignment score)
      USING pixels-shifted alignment offsets per image
      SHIFT image via FFT-based sub-pixel shifting
      RESAMPLE common region of interest from shifted images
```

5. Find peaks via convolution. The locations of fluorescent peptides (peaks) for the first cycle are determined because the signal should be present in at least the initial image to be a valid peptide signal. These peaks correspond to local maxima in signal intensity for each image. To find peaks with 1-pixel accuracy, an approximate point-spread-function kernel (an area under curve of 1.0 Gaussian that has been tuned to match observed empirical data) is convolved with the image in each channel. These locations are then refined to ½ pixel accuracy by using the center-of-mass of the already-identified peaks, determined by the regional context.

```
Pseudo-code:
OUTPUT: peak locations
FOR every channel:
  FIND peaks in cycle 0 by convolving source image with approximate
  PSF
  COMPUTE union of peaks across channels and return as a single list of
  locations
```

6. Fit Gaussian parameters to some or all of the peaks. A subset of the peaks found in step 5 are selected at random and each peak is fit to a 2D Gaussian. This serves to examine peak sizes, potentially at different cycles, as a proxy diagnostic for focus. Although this information is not used in the signal-processing pipeline “proper” (i.e. it is not an input to further downstream processing), it is displayed in reports viewed by persons analyzing the data by default.

7. Compute radiometry parameters per peak. The parameters for the 2D Gaussian point-spread-functions are fit during calibration to the peaks of control peptides located at various positions within the field. As discussed in calibration, the shape and intensity of these point-spread-functions depend on the peptide's location in the field. Appropriate parameters for the PSF are used based on a peak's location in the field to construct the expected PSF image (PSF), which acts as a kernel. This kernel is then convolved with the observed peak background removed image (Data) to derive the signal (Eq. S1b) and noise (Eq. S1e) for every peak in every channel and cycle.

$${σ}_{PSF}^{2} = {\sum }_{i}^{p i x e l s} P S {F}_{i}^{2} \qquad ( S1a )$$

$$signal = \frac{{\sum }_{i}^{pixels} ( {PSF}_{i} * {Data}_{i} )}{{σ}_{PSF}^{2}} \qquad ( S1b )$$

$${e}_{i} = {Data}_{i} - signal * {PSF}_{i} \qquad ( S1c )$$

$${σ}_{e}^{2} = {\sum }_{i} {e}_{i}^{2} \qquad ( S1d )$$

$$noise = \sqrt{\frac{{σ}_{e}^{2}}{{σ}_{PSF}^{2}}} \qquad ( S1e )$$

From the data the number of cycles each peak remains fluorescent is determined.

```
Pseudo-code:
OUTPUT: peak information (radiometry)
FOR each peak
  DEFINE kernel from PSF params based on location in field
  CONVOLVE with peak
  DETERMINE parameters such as signal and noise
```

8. Collation of data into intensity reads and calculation of lifespan metrics. The peak information is collated for the different channels and an intensity array is generated for the channels associated with each peptide termed reads. The data enables one to calculate the lifespan: the number of cycles each peak remains fluorescent. This is calculated from the minimum cosine distance between the measured reads and all possible unit normalized reads. It is the lifespan that is used to calculate the frequency histograms in FIGS. 10, 9, and 17. The intensity summary statistics for each peak during and after its lifetime is additionally calculated.

9. Information of all the identified peaks are collected for each channel across cycles and assembled as an intensity array associated with individual peptides. The peak information for the different channels is collated and an intensity array for the channels associated with each peptide is generated. These intensity reads are then combined into a multidimensional numpy array, called a radmat (radiometry matrix). In the radmat, every row is a peak and every column is the cycle, with signal and noise for the channels occupying different dimensions. A custom python dataframe is used to store information about radmat the other information about the peaks, and a separate data frame for all the metadata about the peak information including: field quality score, field alignment score, aligned position (x and y), and lifespan length. This flexible format allows for a number of computational transformations and extractions, and provides a list of all the information contained in the end of the data.

10. Post Signal Processing Filtering: For all post signal processing analyses, poor quality reads are removed using several filter metrics. First, any fields where the alignment offset is greater than one third of a PSF sub region (150 pixels) are removed. This removes peaks having significant changes in illumination and/or PSF size from cycle to cycle. These extreme misalignments are rare with typical combined offsets between 5 and 25 pixels. Next any field with poor field quality are removed. As discussed above this value measures low frequency (large) structure in the image. Examples of these types of structures include large fluorescent contaminants (e.g., dust, silane clusters, peptide aggregates) or large negative structures (e.g., bubbles). For consistency this value was set to 500 for all runs. To ensure that only single peptides are analyzed, the data is also filtered by how well the peak resembles the expected PSF, for example, low noise values. The most common cause of high noise are non-diffraction limited spots resulting from two or more peptides (or contamination) in close proximity. The noise threshold is chosen to reject above, approximately two standard deviations above mean noise distribution. Because noise and signal are correlated the noise threshold also increases with signal. Currently this threshold is set manually but can be automated to improve reproducibility.

Additional filtering may be used for specific analysis. For colocalization analysis a dark threshold is set at three sigma above the background distribution. For FRET analysis and classification, low intensity contamination is removed by rejecting all peaks above the dark threshold and three sigma below the mean one count intensity. Lastly for classification any high count anomalies at three sigma above the highest count intensity distribution are also removed.

### Estimation of Fluorosequencing Parameters

The fluorosequencing parameters were divided into system-wide parameters and fluorophore-dependent parameters, which are defined in detail in previous publications (see, for example, Swaminathan, Boulgakov, and Marcotte (2015) PLOS Computational Biology 11 (2): e1004080; Swaminathan et al. (2018) Nat. Biotechnol. 36, 1076-1082). They are estimated here through a series of controlled experiments, parameter fitting and estimations. Since the collection of this data automated methods of parameter estimation have been developed (Smith, et al., 2023; Estimating error rates for single molecule protein sequencing experiments. bioRxiv (2023) doi: 10.1101/2023.07.18.549591) based on the whatprot classifier (Smith, Simpson, and Marcotte 2022 PLOS Computational Biology 19 (5): e1011157) and other publicly available data fitting packages. The results in that publication show parameters consistent with the values presented here.

The system-wide parameters include the average probability of Edman failure (p_edman) and surface detachment rate (p_detach). Edman failure is the percent of molecules per cycle that do not undergo the removal of the N-terminal amino acid by Edman degradation. This is modeled in a similar method to that described in Swaminathan et al. (2018) Nat. Biotechnol. 36, 1076-1082. This value is highly dependent on both the experimental conditions and the peptide sequence and ranges from 1 to 20% per cycle. For the improved conditions used in the classification experiments (FIGS. 18 and 21), a value of 5% per cycle was used in the classifier training simulations. During sequencing the entire peptides can be removed by either release of non-specifically bound peptides or hydrolysis of the underlying silane surface. The rate of this detachment from the surface (p_detach) is measured using peptides with two fluorophores and calculating the rate at which the signal for both fluorophores are lost in the same cycle. In the previous publication values of 5% per cycle were reported, here with the surface improvements, this rate is now measured at 0.5% per cycle.

The fluorophore-dependent parameters were determined for each fluorophore, including Alexa555, TexasRed-X, and Atto643 (shown in Table 3). To determine these parameters, controlled experiments with dual-labeled peptides to calculate the surface detachment, above, and the dud-dye rate, and with N-terminally acetylated peptides (JSP260, JSP229, JSP288) which are not subject to Edman degradation chemistry, to isolate losses due to chemical-destruction were performed.

To determine the per cycle photobleaching rate the peptide was continuously illuminated for 120 seconds and an image acquired every second. An exponential decay curve was plotted for the counts of single peptide molecules remaining over time. When acquiring the images, the imaging solvent was used (Table 7). To determine the chemical destruction rate the acetylated peptides were imaged as with fluorosequencing after six Edman cycles and the loss rate of peaks per cycle was measured. This measurement provides the combine per cycle loss due to photobleaching and chemical destruction rates (p_bleach).

TABLE 8
COUNTS OF CLASSIFIED PEPTIDES WITH >0.99
SCORE THRESHOLD

| PEPTIDE CLASS | COUNTS |
|---|---|
| GCKAGAYR (SEQ ID NO: 36) | 1731 |
| GKAGKAGAY (SEQ ID NO: 37) | 1608 |
| GKAGCAGSY (SEQ ID NO: 38) | 1012 |
| GKGASCGANSAKGSYRN | 557 |
| (SEQ ID NO: 39) | |
| KSPVSLAQYWWNQINPFYQM | 14 |
| (SEQ ID NO: 40) | |
| RGAWMKRYKGAWPQWDANCS | 11 |
| (SEQ ID NO: 41) | |
| FSQQETECKYMPFPARNLDM | 4 |
| (SEQ ID NO: 42) | |
| SCKPARPKGFLQHPWDYAPD | 4 |
| (SEQ ID NO: 43) | |
| MDKYHNVQWPCIFAPAGRKC | 2 |
| (SEQ ID NO: 44) | |
| CQVRHCFGSMQDMWHPIAHM | 2 |
| (SEQ ID NO: 45) | |
| PQMLTDSATKKPFDVVHIPF | 2 |
| (SEQ ID NO: 46) | |
| TPCFGEKYANVGDHTWWMHW | 1 |
| (SEQ ID NO: 47) | |
| | 4948 |

As described previously, it was observed that peptides with two counts of the same fluorophores, which was confirmed through mass characterization, appeared to have the brightness of only a single fluorophore. It is speculated that the sample preparation process could have caused photobleaching or the formation/presence of a non-fluorescing isomer. To calculate the dud dye rate (R_{dd}, p_dud), multiple fields of the dual-labeled peptide were imaged. By measuring the count of peptides with one (Fₛ) and two (F_{d}) fluorophores, the fraction of fluorophores that are considered “duds” was computed using the Eq. S2. This calculation will slightly underestimate the true dud dye 10 rate as the fraction of peptides with no fluorescence was not able to be calculated.

$${R}_{d d} = \frac{{F}_{s}}{2 ( {F}_{S} + {F}_{d} )} \qquad ( S2 )$$

Lastly, the mean intensity distribution and its standard deviation are calculated for the population of peptides with single fluorophore fluorescence.

### Building Machine Learning Classifier

To infer peptide identity, a workflow was designed involving building a machine learning classifier which classifies and scores the signal data obtained from fluorosequencing directly to peptide identity.

1. Reference peptide database is created. An expected peptide database was created either from a protein list, simulating the peptides generated from protease digestion in the sample, or by generating directly from a list input peptides. In the case of building the four peptide classifier FIG. 18, a random set of 50 peptides was also included as a decoy list. In the case of the MHC peptide experiment, reference peptides identified using mass-spectrometry were used as the reference database. The peptide sequences were then converted to a “fluorostring” represented as [ . . . 0.1 . . . 1] where “.” represents an unlabeled amino acid. The numbers represent the fluorophores for each channel (0 or 1).

2. Using Monte Carlo simulations, synthetic fluorosequencing reads are generated for reference database peptides. Using the experimentally obtained fluorosequencing parameters detailed above, Monte-Carlo simulations were performed for each peptide in the list by simulating 1000 copies for each peptide, labeling the selected amino acids with fluorophores, and using the probability of Edman failure, dud-dye, photobleaching and dye-destruction, from the experimentally obtained fluorosequencing parameters detailed above, for the simulation. At each cycle, the possibility of Edman failure, dye bleaching and so on, was simulated to arrive at a dye-sequence for the peptide. The resulting sequence is assigned a random value drawn from the intensity distribution for the channel dye, yielding the signal for the peptide at each cycle. Note that the information about the originating peptide is stored alongside the simulated fluorosequencing read. The sequence of radiometry at each cycle, in each channel, for each peptide follows the same format as the data produced by the instrument through the signal processing pipeline described above.

3. A random forest classifier is trained on the synthetic reads. The peptide/fluorosequencing data generated from Monte-Carlo simulation was used to construct a multi-class Random forest classifier. The number of features employed was determined by multiplying the number of channels and the number of cycles. Typically, the training set comprised 80% of the data, while the remaining 20% was reserved for testing.

Raw intensity array data for each individual peptide obtained from fluorosequencing experiment is scored against the random forest Classifier. The machine learning classifier was used to classify and score the intensity array (reads) generated from signal processing steps for each read. The classifier assigns a score to all peptide classes for each read, which can be considered a probability of assigning the read to the correct peptide class. The read is then attributed to the highest scoring peptide class. To determine a scoring threshold, the scores associated with the decoy peptide list (known to be incorrect classifications) and the scores associated with the input peptides (known to be correct) are examined. In the case of the four peptide mixture samples (FIG. 18), a score threshold of 0.99 was applied and the counts for the different classified peptides were obtained. In the case of the MHC peptide mixture samples (FIG. 21), a score threshold of 0.7 was applied and the counts for the different classified peptides were obtained. For the high-scoring reads above, the reads were clustered using the Python umap-learn package's default settings, indicating each read's assignment by color.

### Example 10

### Synthesis of Polymers (or “Promers”)

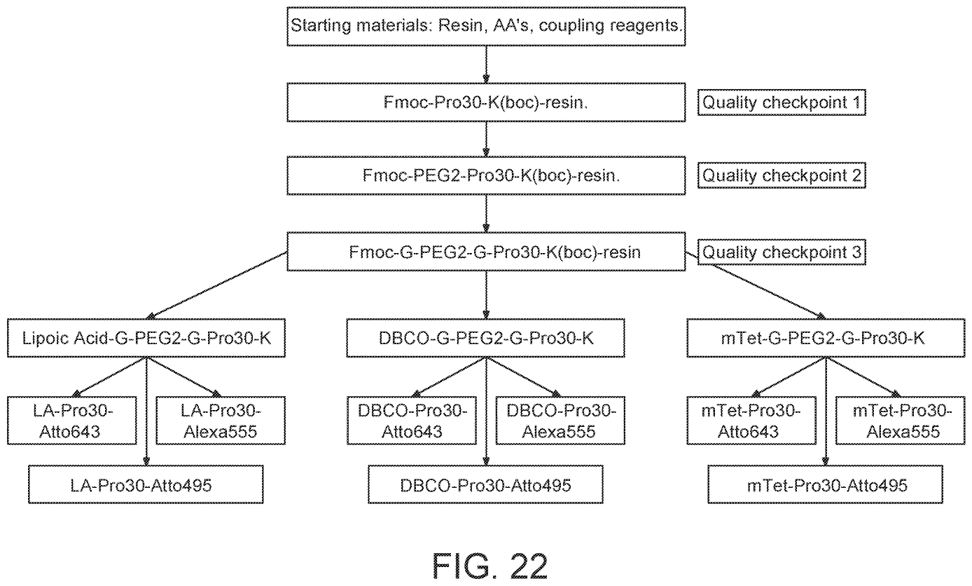

The preferred method of synthesis of a polymer (or “Promer”; polyproline) is through the use of solid phase peptide synthesizer. The section below describes the detailed protocol for synthesis of a polyproline polymer and the methods used for validation. The workflow for polymer (or “promer”) synthesis and installation of functional group and fluorophore is shown in FIG. 22.

### Experimental Procedure for Synthesis

### Overview

A series of bioorthogonal reactions are performed to selectively label amino acids. Covalent attachment of fluorophores to amino acids occurs through a Proline linker, obtained from Fmoc-G-PEG₂-G-Pro30-K(boc)-resin.

This Example involves the synthesis of Fmoc-G-PEG₂-G-Pro30-K(boc)-resin. The steps to the synthesis are detailed in the experimental protocol below.

### Aim:

To load a solid phase support with the peptide-Fmoc-G-Peg₂-G-Pro (30)-K(boc)-Resin

### Materials:

1. Tentagel S Ram: Millipore Sigma; SKU 86407-5G
2. Diisopropylcarbodiimide (DIC): CAS #693-13-0; Millipore Sigma Cat #
3. Ethyl isonitrosocyanoacetate (Oxyma): CAS #3849-21-6; Millipore Sigma; Cat
4. Fmoc-Peg2-COOH: CAS #791028-27-8; Broadpharm; Cat #BP-20523
5. Fmoc-Pro-COOH: CAS #71989-31-6; Fisher Scientific
6. Fmoc-Gly-COOH: CAS #29022-11-5; Millipore Sigma
7. Fmoc-Lys(boc)-COOH: CAS #71989-26-9; Millipore Sigma
8. Fritted syringe: CAT #NC9299152; Fisher Scientific
9. Acetonitrile: CAS #75-05-8; Millipore Sigma
10. Methanol: CAS #67-56-1; Millipore Sigma
11. DCM: CAS #75-09-2; Millipore Sigma
12. DMF: CAS #68-12-2; Millipore Sigma
13. Piperidine: CAS #110-89-4; Millipore Sigma
14. Triisopropyl Silane (TIPS): CAS #6485-79-6; Millipore Sigma
15. Trifluoroacetic acid (TFA): CAS #76-05-1; Millipore Sigma
16. Deionized water

### Solutions Prepared:

1. 0.2 M Fmoc-PEG2-COOH in DMF
2. 0.2 M Fmoc-Pro-COOH in DMF
3. 0.2 M Fmoc-Gly-COOH in DMF
4. 0.2 M Fmoc-Lys(boc)-COOH in DMF
5. 1 M DIC in DMF
6. 1 M Oxyma in DMF
7. deFmoc solution: 20% piperidine in DMF (v/v)
8. TFA Cocktail: 95% TFA, 2.5% TIPS, 2.5% H2O (v/v)

### Instrument/Consumable Used:

1. Peptide synthesizer: CEM Liberty Blue microwave peptide synthesizer
   a. Model No.: 909410
2. Vacuum manifold
3. Fritted syringe: CAT #NC9299152; Fisher Scientific

### Abbreviations:

1. RV=Reaction vessel
2. HS=High swelling
3. DMF-Dimethylformamide
4. TFA=Triflouroacetic acid
5. TIPS=Triisopropyl silane
6. RT=room temperature
7. DCM=Dichloromethane
8. MeOH=Methanol
9. ACN=Acetonitrile
10. RPM=Rotations per minute

### Procedure:

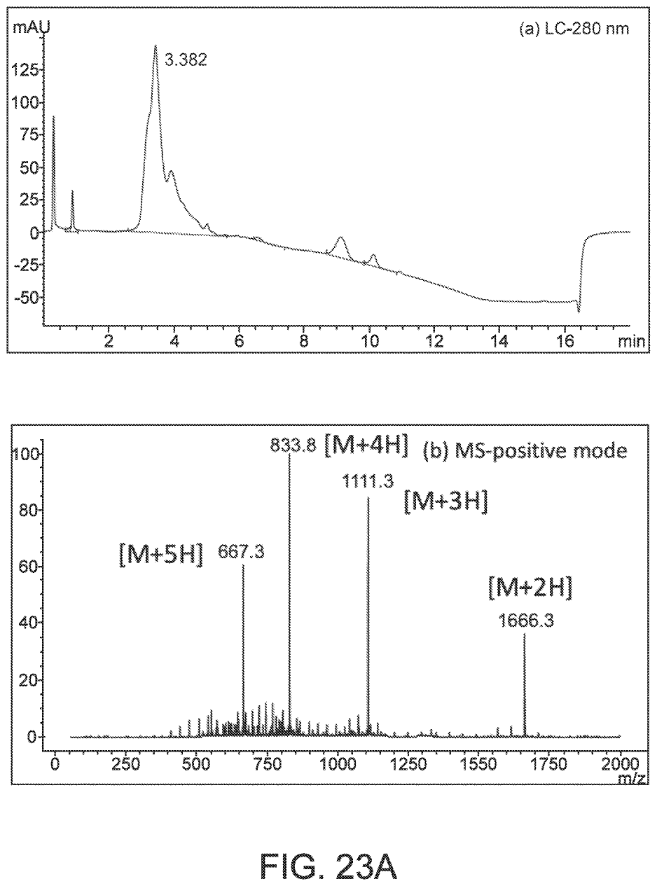

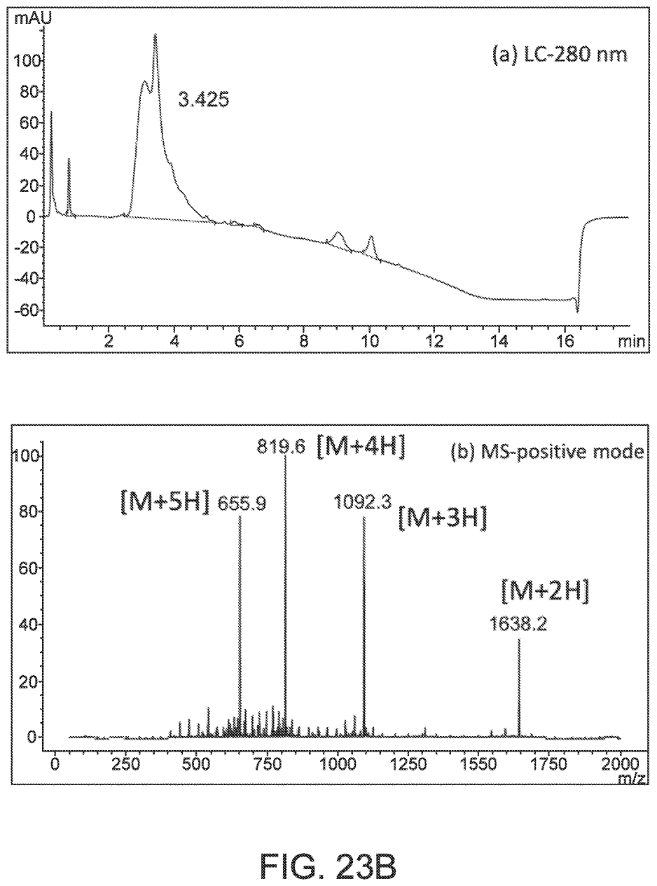

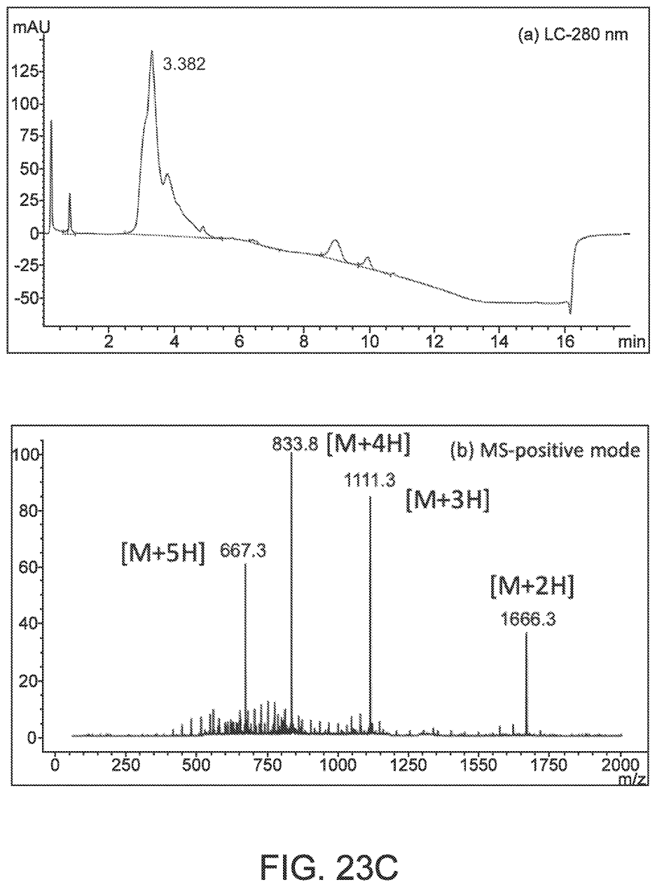

1. Synthesis of Fmoc-Pro20-K(boc)-CO-Rink-Resin performed on Peptide synthesizer with the following protocol:
   a. Standard deprotection
      i. Add deprotection solution
      ii. 75° C. using 175 Watts for 15 s
      iii. 90° C. using 30 Watts for 50 s
      iv. Drain reaction vessel (RV)
      v. Wash for 10 seconds using DMF
   b. 1.25 mmol single coupling (HS)
      i. Standard deprotection
      ii. Wash with DMF (3×)
      iii. Drain RV
      iv. Couple amino acid
2. Extending resin to Fmoc-Pro30-K(boc)-CO-Rink-Resin
   a. Double coupling protocol (HS) used
      i. Standard deprotection
      ii. Wash with DMF (3×)
      iii. Drain RV
      iv. Couple amino acid (2×)
3. QC checkpoint—A (FIG. 23A)
   a. ˜1 mg of resin is used to perform a test cleavage (see details and metrics in procedure below).
   b. Cleavage conditions: 95% TFA; 2.5% TIPS; 2.5% H20 for 1 h at RT
4. Extension to Fmoc-Peg2-G-CO-Rink-Resin
   a. Triple coupling protocol (HS) used
      i. Standard deprotection
      ii. Wash with DMF (3×)
      iii. Drain RV
      iv. Couple amino acid (3×)
5. QC checkpoint—B (FIG. 23B)
6. Extension to Fmoc-G-Peg2-G-CO-Rink-Resin
   a. Standard deprotection
   b. Wash with DMF (3×)
   c. Drain RV
   d. Couple amino acid (3×)
7. QC checkpoint—C(FIG. 23C)
8. Beads are transferred to a clean Fritted syringe
9. Washing of beads via gravity-drip method
   a. DMF (3×)
   b. DCM (3×)
   c. MeOH (3×)
10. Drying and storing
   a. Following wash, vacuum dry beads
   b. Store in −20° C.

### Protocols for Validating Polymer (or “Promer”) Synthesis

As shown in FIGS. 23A-23C, the three checkpoints validating polymer synthesis are highlighted and protocols described below.

### Quality Control Checkpoints:

1. QC checkpoint—A: Following the initial synthesis of Fmoc-G-P30-K(boc)-resin
   a. Metric: Fmoc-G-P30-K-ONH₂ is detected via LC-MS ESI mass spectrometry
2. QC checkpoint—B: Following the triple coupling of the PEG₂ residue
   a. Metric: No species corresponding to NH₂-G-P30-G-K-ONH₂ is detected via mass spectrometry upon test cleavage
3. QC checkpoint—C: Following the final coupling of Fmoc-Gly-OOH
   a. Metric: synthesis of desired product at >70% composition via LC/MS

### Materials:

1. Triisopropyl Silane (TIPS): CAS #6485-79-6; Millipore Sigma
2. Trifluoroacetic acid (TFA): CAS #76-05-1; Millipore Sigma
3. Diethyl ether: CAS #60-29-7; Millipore Sigma
4. Acetonitrile: CAS #75-05-8; Millipore Sigma
5. Deionized water
6. Fritted syringe: CAT #NC9299152; Fisher Scientific

### Solutions Prepared:

1. Cleavage solution: 95% TFA/2.5% TIPS/2.5% H20 (v/v) 2. 50% ACN/50% H20 (v/v)

### Instrument/Consumable Used:

1. LCMS equipment
   a. Agilent 1260 Infinity Degasser (G1322A)
   b. Agilent 1260 Infinity Binary Pump (G1312B)
   c. Agilent 1260 Infinity Sampler (G1329B) with external tray (p/n G1313-60004) and waste tube (p/n G1313-27302)
   d. Agilent 1260 Infinity Column Thermostat (G1316A) with a 2-position/6-port valve
   e. Agilent 1260 Infinity Diode Array Detector (G4212B)
   f. Agilent 6120B Single Quadrupole LC/MS (G6120B) with multimode source enabled with fast polarity switching for positive and negative mode acquisitions
   g. Agilent ZORBAX Eclipse Plus C18 narrow bore column; 2.1 mm internal diameter; 50 mm length; 5 micron particle size; P.N. 959746-902.
2. Fritted syringe: CAT #NC9299152; Fisher Scientific

### Procedure:

1. Prepare a cleavage solution with the following composition by volume:
   a. TFA (95%)
   b. TIPS (2.5%)
   c. H20 (2.5%)
2. Add cold 1 mL diethyl ether to −80° C.
3. Weigh out 1 mg of the resin loaded with the target peptide.
4. Submerge the beads in 100 μL of the cleavage solution.
5. Agitate the mixture at RT for 2 hours.
6. Using an inert gas, gently blow-dry the cleavage solution.
7. Once dry, precipitate out the cleaved peptides using the cold ether prepared prior.
8. Centrifuge the solution at 25,000 RPM for 10 minutes.
9. Decant the ether from the precipitated pellet.
10. Solubilize the pellet in a solution of 25 μL of ACN and 25 μL of H20.
11. Analyze via LC/MS (see QC results below).

### Quality Control Results (Representative Examples)

### Quality Checkpoint a

### Checkpoint Notes:

1. The purpose of this checkpoint is to ensure that the major product of synthesis is Fmoc-Pro30-K(boc)-resin as opposed to a twenty-nine-mer and other smaller sequences.
2. The mass spectra shown in FIG. 23A (bottom) demonstrates a common result produced by ESI mass spectrometry. A majority signal corresponding to the successful coupling of 30 prolines is present along with lower intensity signals of peptides of shorter sequence.
3. Failed couplings will occur during synthesis, this is expected and accepted. However, the thirty-mer should be the major product.

### Metric:

The synthesis of Fmoc-Pro30-K(boc)-ONH₂ should be successful, and the thirty-mer should be the dominant product.

### QC Fail Solution.

Repeat the necessary amount of couplings to achieve a thirty-mer.

### Quality Checkpoint B:

(a) FIG. 23B (top) shows a liquid chromatogram showing the results of a test cleavage performed following the synthesis of Fmoc-PEG₂-G-Pro30-K(boc)-ONH₂ after employing a triple coupling. (b) The resulting ESI mass spectra (FIG. 23B (bottom)) from integrating the area under the curve from 2.5-5 minutes on the tandem mass spectra. This mass spectra reveals the coupling of Fmoc-PEG₂-COOH goes to completion after utilizing a triple coupling.

### Checkpoint Notes:

1. The purpose of this checkpoint is to ensure that the coupling of Fmoc-PEG₂-COOH goes to completion.
2. The coupling of Fmoc-PEG₂-COOH has been found to occasionally fail when double couplings are employed. This produces a secondary product that interferes with our downstream measurements. Utilizing a triple coupling method has been shown to reproducibly mitigate this error, and increases sample homogeneity and purity.

### Metric:

The coupling of Fmoc-PEG₂-COOH should go to completion as suggested by mass spectrometry and liquid chromatography.

### QC Fail Solution

Repeat the coupling to improve sample homogeneity.

### Quality Checkpoint C

(a) FIG. 23C (top) shows liquid chromatography results from the coupling of Fmoc-G-COOH to the loaded resin. (b) FIG. 23C (bottom) shows tandem ESI mass spectra integrated from 2.5-5 minutes, suggesting the major product of synthesis to be Fmoc-G-PEG₂-G-Pro30-K-ONH₂.

### Checkpoint Notes:

1. The purpose of this checkpoint is to ensure that the final desired product is the major species on resin.
2. This coupling is very robust. Triple coupling is recommended.

### Metric:

The final major product of synthesis should correspond to a peptide with the sequence Fmoc-G-Pro30-G-K(boc)-resin as read from the N terminus to the C terminus from left to right. This is determined via liquid chromatography and mass spectrometry.

### QC Fail Solution:

Repeat final coupling until desired product is achieved.

### Functionalization of “Click” Reactive Group on the Polymer

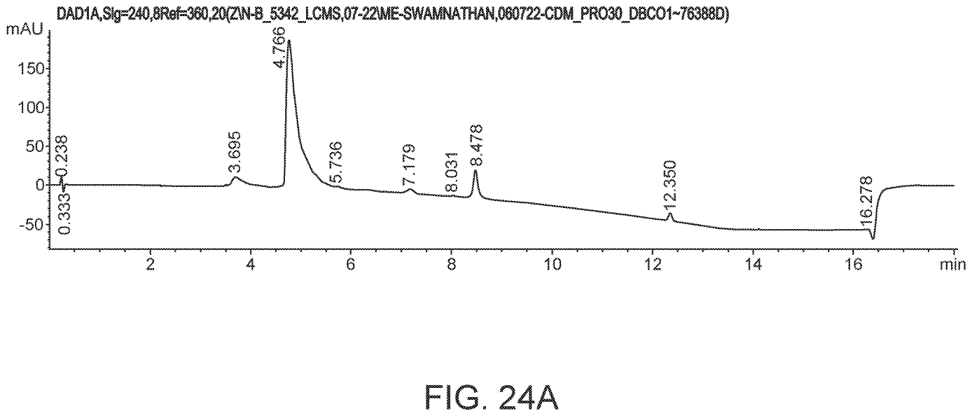

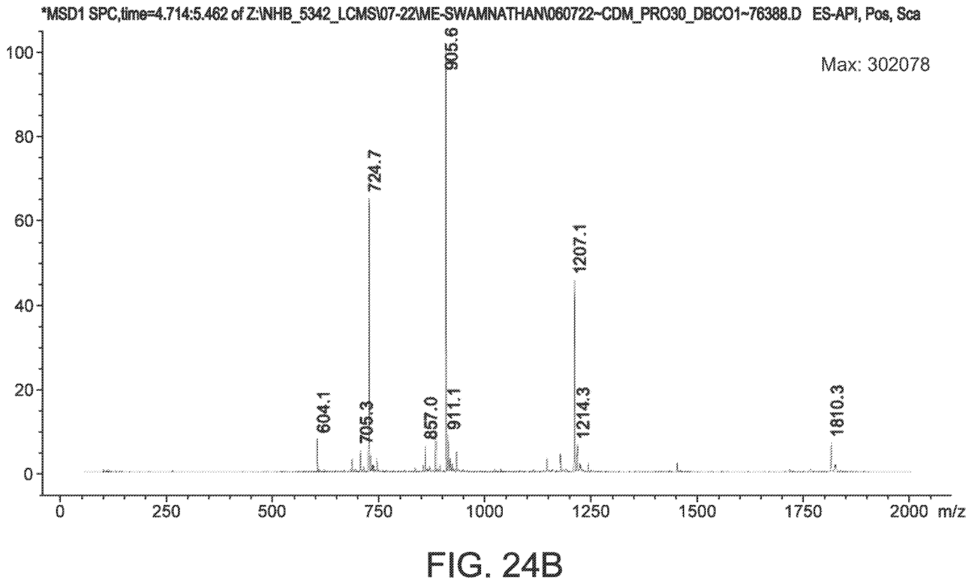

As an example, the below describes the procedure used for functionalizing the polymer with DBCO. LC-MS data validates the synthesis and purification of the click functionalized polymer. Similar methods may be adapted for functionalization of methyltetrazine, lipoic acid or other similar reactive groups. FIGS. 24A-24B shows validation of the DBCO functionalized polymer (or “promer”).

### Aim:

To conjugate a functional handle to a polymer that is resin-bound via the C-terminus.

### Materials:

1. Fmoc-G-PEG2-G-Pro30-resin: Source N/A
2. DBCO-NHS; CAS #1353016-71-3; Broadpharm; Cat #BP-22231
3. HEPES buffer 1.0 M pH 7.5; CAT #15630106; Thermo Fisher Scientific
4. Acetonitrile: CAS #75-05-8; Millipore Sigma
5. DMF: CAS #68-12-2; Millipore Sigma
6. Methanol: CAS #67-56-1; Millipore Sigma
7. DCM: CAS #75-09-2; Millipore Sigma
8. Piperidine: CAS #110-89-4; Millipore Sigma
9. Diethyl Ether: CAS #60-29-7; Millipore Sigma
10. Fritted syringe: CAT #NC9299152; Fisher Scientific
11. Triisopropyl Silane (TIPS): CAS #6485-79-6; Millipore Sigma
12. Trifluoroacetic acid (TFA): CAS #76-05-1; Millipore Sigma
13. Deionized water

### Solutions Prepared:

1. deFmoc solution: 20% piperidine in DMF (v/v)
2. 0.2 M DBCO-NHS in 50/50 ACN/HEPES buffer (0.1 M pH 7.5) (v/v)
3. TFA cleavage solution: TFA/TIPS/H20 95/2.5/2.5 (v/v)

### Instrument/Consumable Used:

1. Vacuum manifold
2. Fritted syringe: CAT #NC9299152; Fisher Scientific

### Procedure:

1. Standard deprotection of Fmoc-G-PEG2-G-Pro30-K(boc)-resin.
   a. Add resin to fritted syringe and cap
   b. Add deprotection solution and seal vessel
   c. Incubate for 1 hour
   d. 37° C.
2. Standard wash of resin
   a. DMF 3× via gravity drip wash
   b. MeOH 3× via gravity drip wash
   c. DCM 3× via gravity drip wash
   d. Vacuum dry resin
3. Conjugation of DBCO-NHS to G-PEG2-G-Pro30-K(boc)-resin
   a. Submerge beads in fresh anhydrous DMF
   b. Add 5 equivalents (˜0.5 mmol per 4 grams of loaded resin) of DBCO-NHS solution to the submerged beads
   c. Incubate for 2 hours
   d. 37° C.
4. Standard wash of resin
   a. DMF 3× via gravity drip wash
   b. MeOH 3× via gravity drip wash
   c. DCM 3× via gravity drip wash
   d. Vacuum dry resin
5. TFA cleavage of DBCO-G-PEG2-G-Pro30-K from resin
   a. Cool and store at least 10 mL of diethyl ether to −80° C.
   b. Submerge beads in TFA cleavage solution and seal vessel
   c. Incubate for 2 hours at room temperature
   d. Decant cleavage solution from beads
   e. Blow dry cleavage solution using nitrogen in a well ventilated fume-hood
   f. Add cold ether from (a) to the dried cleavage vessel to precipitate desired product
   g. Centrifuge the mixture
   h. 20817 RPM
   i. 4° C.
   j. 15 minutes
   k. Decant ether
   l. Solubilize pellet in 50/50 ACN/H2O
6. HPLC purify product

### Functionalization of Fluorophore

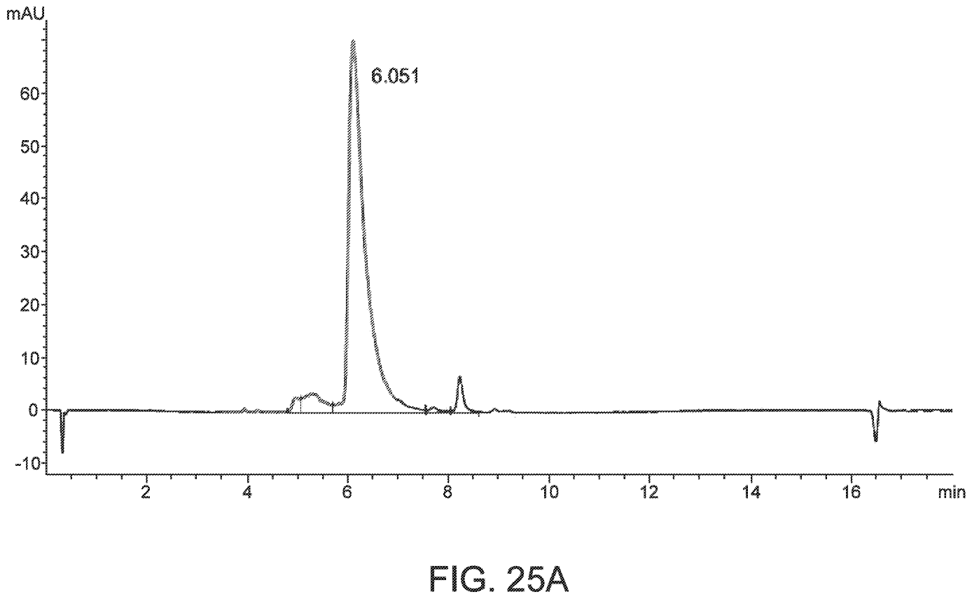

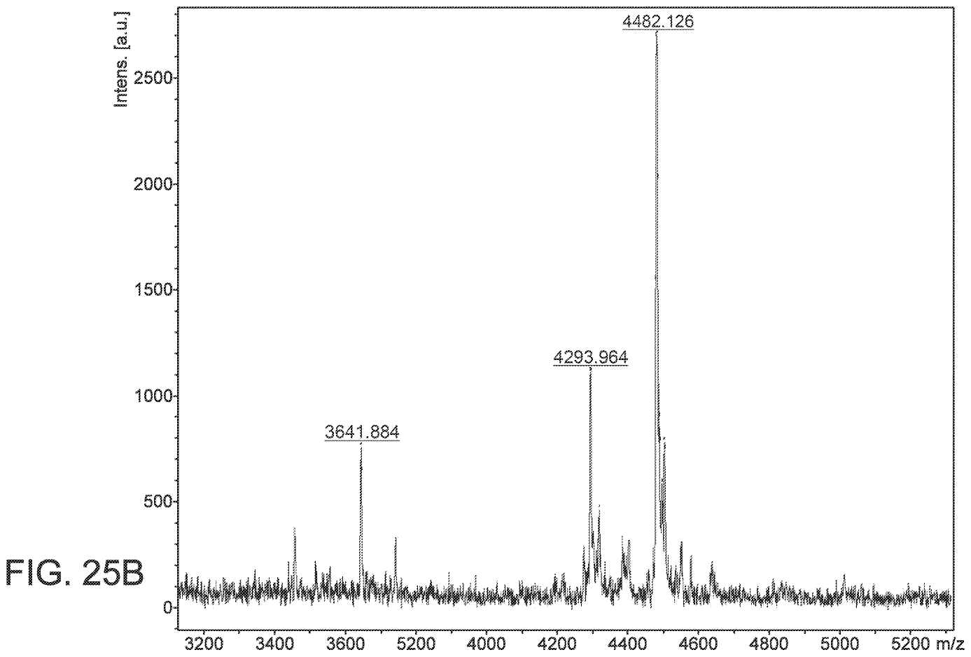

As an example, Atto643 fluorophore may be installed on the other end of the polymer. A representative trace for HPLC purification and mass spectrometry analysis is shown in FIGS. 25A-25B.

### Aim:

To conjugate a fluorophore to a polymer via the side chain of lysine in the polymer sequence.

### Materials:

1. HEPES buffer 1.0 M pH 7.5; CAT #15630106; Thermo Fisher Scientific
2. Acetonitrile: CAS #75-05-8; Millipore Sigma
3. Atto643-NHS ester: PN #AD 643; ATTO-TEC
4. Deionized water

### Solutions Prepared:

1. Polymer solution: 0.1 μM in 50/50 ACN/HEPES buffer (0.1 M pH 7.5) (v/v)
2. Dye solution: 5.0 μM in 50/50 ACN/HEPES buffer (0.1 M pH 7.5) (v/v)

### Procedure:

1. Conjugation of Atto643-NHS to DBCO-G-PEG2-G-Pro30-K-ONH2
   a. Add 1.1 equivalents of polymer solution to the dye solution
   b. Incubate for 2 hours
   c. 37° C.
2. HPLC purify product

### Example 11

### Example Uses of Polymers (or “Promers”)

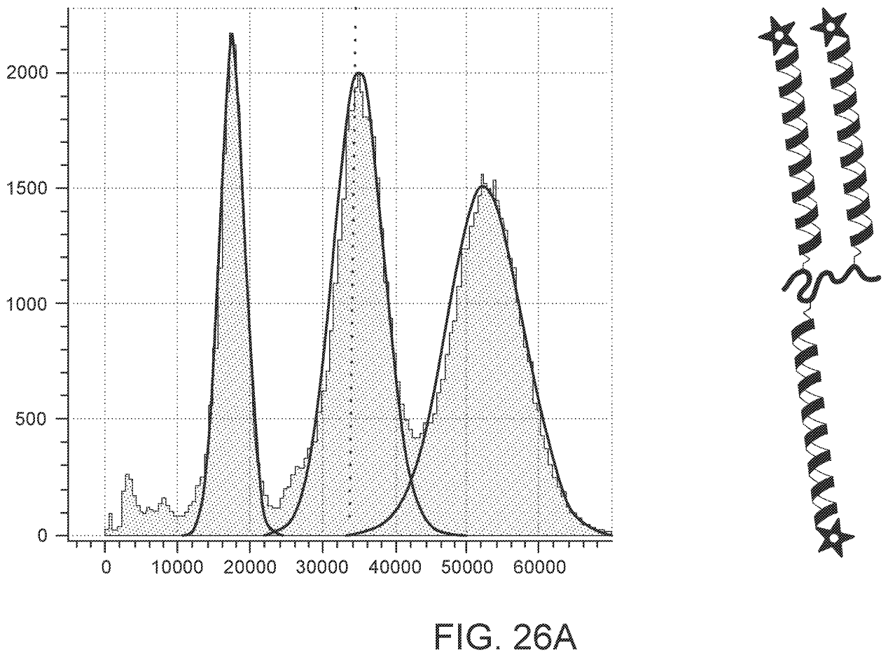

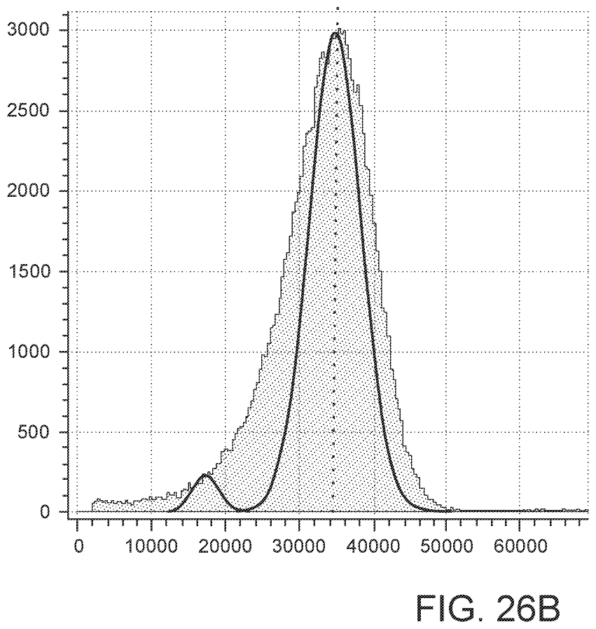

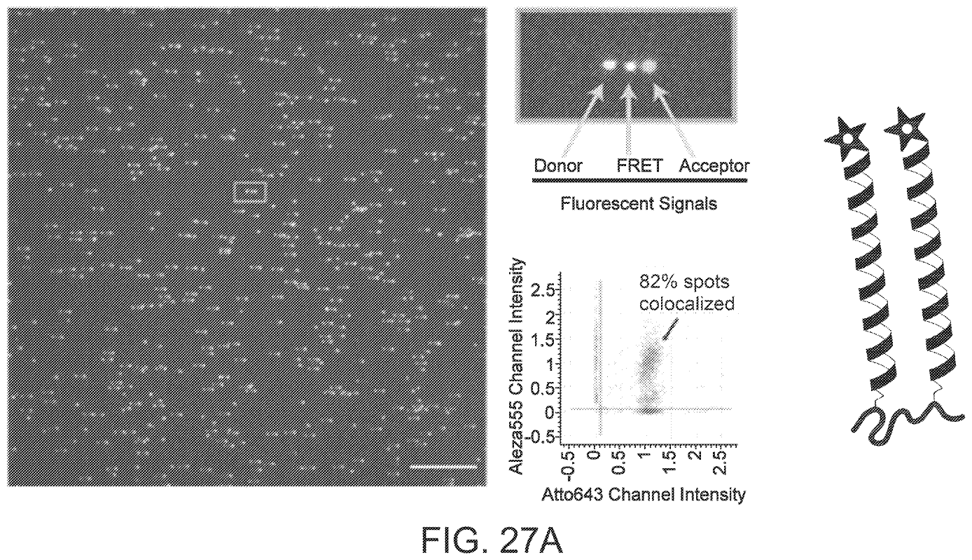

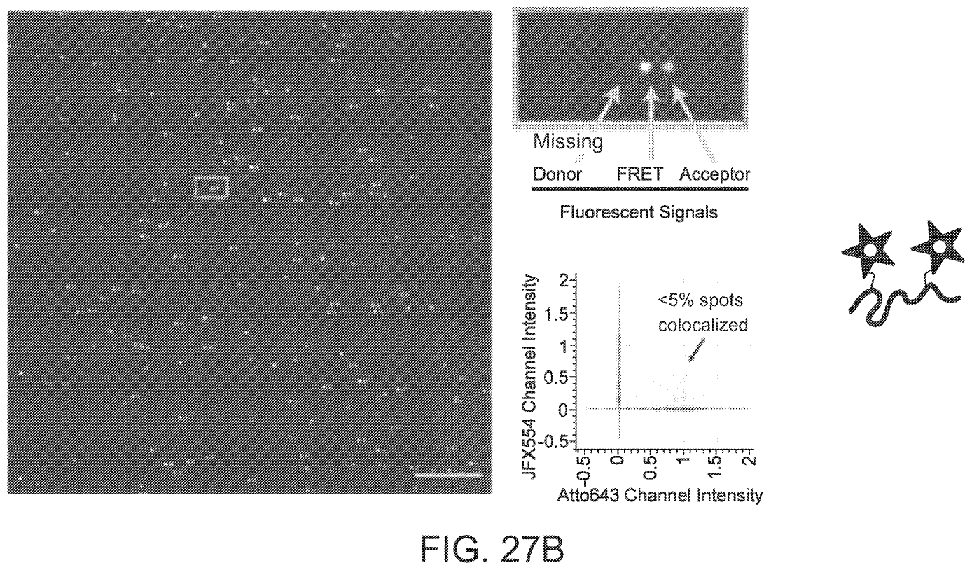

The polymers (or “promers”) react to amino acid side chains (that are converted to the complementary click partner; for example if the end functional group on the polymer, or “Promer” is DBCO, then the amino acid side chain on peptide is converted to an azide) forming a hybrid biomolecule with long rigid polymers grafted on to the peptide. Mitigation of dye-dye interactions is observed through installing multiple promers on the same peptide molecule. The presence of the rigid rod and the separation of dyes is hypothesized to produce reduction of quenching between same fluorophores or FRET for dissimilar and spectrally overlapping fluorophores. The effects of this are visible on single molecule microscopy experiments and can be seen in FIG. 26 (quenching) and FIG. 27 (FRET).

### Experiment Details:

### Installing Functionalized Polymers (or “Promers”; DBCO-Pro30-ATTO643) on an Azidolysine Peptide

30 nmol of K₂azK₃ was dissolved in 30 uL 1:1 ACN:HEPES (50 mM, 7.5 pH HEPES). To this 31.5 nmol (1.05 eq) of DBCO-G-PEG₂-G-P30-K[Atto643]-CONH₂, dissolved in 30 uL of 1:1 ACN:HEPES (50 mM, 7.5 pH HEPES), was added. Solution was agitated at 40 C. After 5 hrs, 1M 8.5 pH HEPES buffer was added to bring the measured pH to 8.5. NHS-PEG₄-N₃ (90 nmol, 3 eq) dissolved in minimal CH₃CN was added. Solution was agitated for 2 hrs at 40° C. The sample was then HPLC purified and the major product (K₂[NHS-PEG₄-N₃]azK₃[DBCO-G-PEG₂-G-P30-K[Atto643]-CONH₂]) confirmed by LCMS. Following purification, the product yield was determined using UV-vis (643 nm abs max) and 2 eq of DBCO-G-PEG₂-G-P30-K[JF549]-CONH₂ was added and solution agitated at 40 C. Following 12 hrs the sample was HPLC purified and the major product characterized as (K₂[(NHS-PEG₄-N₃) DBCO-G-PEG₂-G-P30-K[JF549]-CONH₂]azK₃[DBCO-G-PEG₂-G-P30-K[Atto643]-CONH₂]) using LC-MS and MALDI. SDS PAGE was used to determine the relative mass of the labeled peptide in reference to other “polymer labeled peptides” (see example). TIRF microscopy was used to characterize the fluorescent intensity of the JF549 and Atto643 dyes. The peptide was stored at −80° C. in 7.5 pH HEPES/ACN and used for TIRF microscopy.

### Example 12

### Conjugation of NHS-DBCO to Non-Functionalized Polyproline

### Aim:

To conjugate DBCO-NHS to the non-functionalized polymer (or “Promer”). The final molecule being a functionalized peptide backbone with an amino-acid based linker molecule and sequence from N terminus to C terminus being the following:

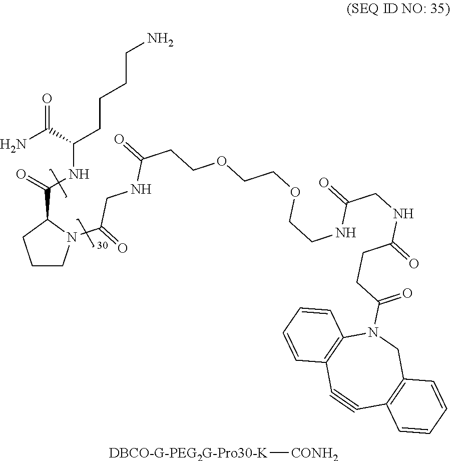

### Materials:

1. Fmoc-G-PEG₂-G-Pro30-K(boc)-resin//CASLO
2. DBCO-NHS//BP-22231//Broadpharm
3. TFA//302031//Sigma Aldrich
4. TIPS//233781//Sigma Aldrich
5. TEA//471283//Sigma Aldrich
6. DCM//34856//Sigma Aldrich
7. Piperidine//104094//Sigma Aldrich
8. Diethyl ether//296082//Sigma Aldrich
9. HEPES buffer solution 1.0 M pH 8.5//J61360//Alfa Aesar
10. Acetonitrile//271004//Millipore Sigma
11. Formic acid//AC270480250//Fisher Scientific
12. Ammonium carbonate//036229-36//Thermo Scientific
13. Water

### Solutions Prepared:

1. 2.0 M NHS-DBCO in DMF
2. 20% piperidine in DMF=deprotection solution
3. 47.5/47.5/2.5/2.5→TFA/DCM/TIPS/H₂O (v/v)=cleavage solution

### Instrument/Consumable Used:

1. Eppendorf Thermomixer C Model #: 5382
2. LC/MS instrumentation
   a. Agilent 1260 Infinity Degasser (G1322A)
   b. Agilent 1260 Infinity Binary Pump (G1312B)
   c. Agilent 1260 Infinity Sampler (G1329B) with external tray (p/n G1313-60004) and waste tube (p/n G1313-27302)
   d. Agilent 1260 Infinity Column Thermostat (G13 16A) with a 2-position/6-port valve
   e. Agilent 1260 Infinity Diode Array Detector (G4212B)
   f. Agilent 6120B Single Quadrupole LC/MS (G6120B) with multimode source enabled with fast polarity switching for positive and negative mode acquisitions
   g. Agilent ZORBAX Eclipse Plus C18 narrow bore column; 2.1 mm internal diameter; 50 mm length; 5 micron particle size; P.N. 959746-902.

### Procedure:

1. Begin cooling diethyl ether to −80° C.
2. Weigh out 3 g of resin loaded with the Fmoc-G-PEG₂-G-Pro30-K peptide.
3. Submerge resin beads in the deprotection solution for 30 minutes at 40° C. 2-3× the volume of DMF may be needed due to resin swelling.
4. Wash the beads 3× with each of the following solvents: DMF→DCM→MeOH.
5. Carefully dry the beads with a vacuum manifold.
6. Add 2.0 M DBCO-NHS to the beads, being mindful to add a total of 1 gram to the beads. Dilute the reaction with DMF to completely submerge the beads in the reaction solution.
7. Add 300 uL of TEA to the reaction vessel.
8. Allow the reaction to reach equilibrium over 2 hours at 37° C.
9. Wash the beads 2× with each of the following solvents: DMF→DCM→MeOH
10. Carefully dry the beads with a vacuum manifold
11. Completely submerge the beads in the cleavage solution. Leave the cleavage reaction to reach equilibrium at room temperature for 2 hours.
12. Filter the cleavage solution into a separate vessel. Wash the beads 1× with DCM, and collect the wash into the cleavage solution.
13. Note the total volume of collected cleavage solution, and dry the solution down to ≤5% of the original volume.
14. Add cold diethyl ether to precipitate the target peptide. Decant the solution from the pellet.
15. Neutralize any residual acid with an LC/MS compatible base.
16. Upon completion of the reaction, purify the sample via HPLC.
   a. Solvent A: 0.1% FA in H2O
   b. Solvent B: 0.1% FA in ACN
   c. Flow rate: 10 mL/min
   d. Column: Jupiter 5 μm C18, 300 A, 250×21.1 mm
   e. Gradient.

| Time (min) | Solvent B % |
|---|---|
| 0.01 | 5 |
| 10.0 | 5 |
| 15.0 | 28 |
| 18.0 | 32 |
| 19.0 | 35 |
| 20.0 | 35 |
| 25.0 | 50 |
| 30.0 | 70 |
| 35.0 | 80 |
| 45.0 | 95 |
| 55.0 | 95 |

17. Immediately following elution from the HPLC, titrate the purified sample to neutral pH using ammonium carbonate. (See supplementary information below for further information.)

### Supplementary Information

### DBCO Degradation and Mitigation.

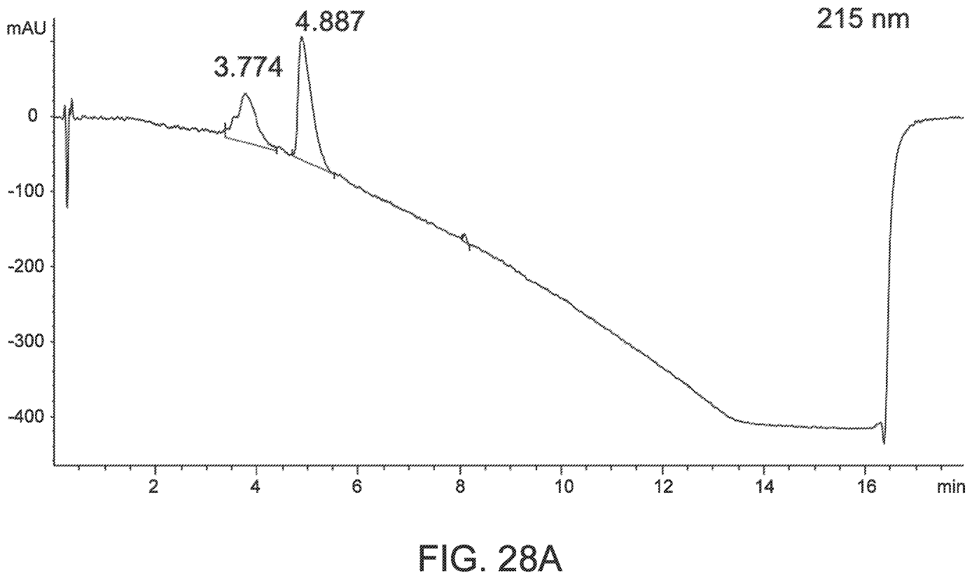

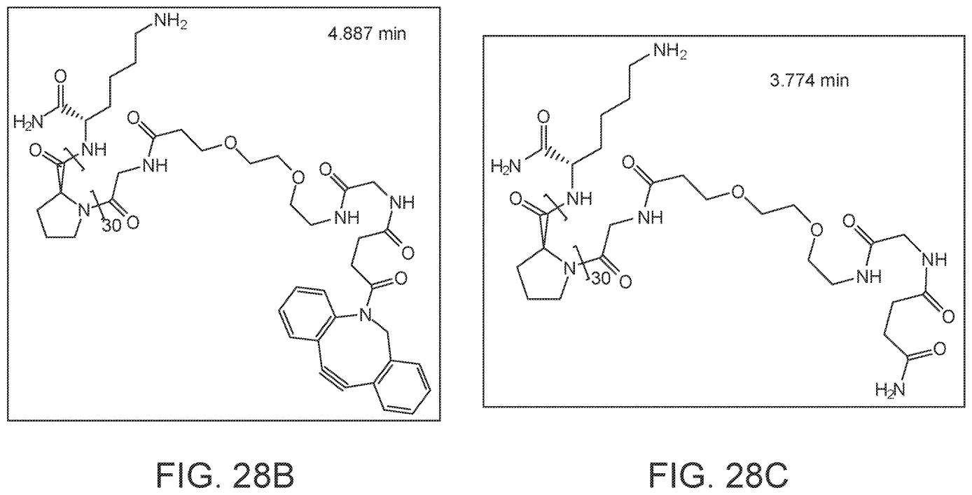

It is believed that the DBCO functional group undergoes a 5-endo-dig cyclo-isomerization to form a small heterocycle in acidic conditions (seen at 3.774 minutes, FIGS. 28A and 28C). This issue primarily interferes with HPLC purification.

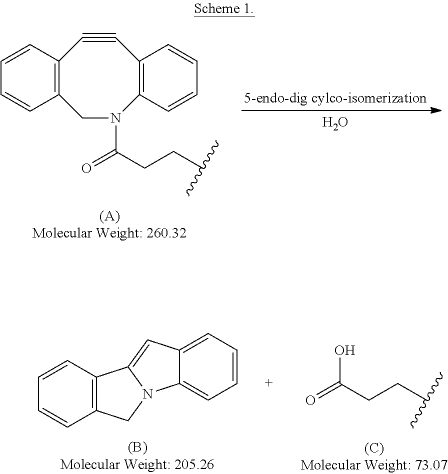

In order to mitigate this problem, neutralization via ammonium carbonate was employed following purification.
Neutralization via HEPES buffer was also attempted. However, the addition of HEPES prior to lyophilization caused difficulties with solubility and salt concentrations. Ammonium carbonate was used due to its greater volatility.
In spite of its greater volatility, the resulting ammonium formate is typically only removed via lyophilization. If lyophilization is not an option, then ammonium carbonate should not be used. The resulting ammonium formate salt reacts nearly instantly with NHS esters, resulting in the formation of an non-reactive amide bond.

### Mass Spectra Trends and Analysis

The backbone of the polymer (or “promer”) technology is developed by utilizing solid phase peptide synthesis methods. Achieving sample homogeneity on solid-phase supports becomes increasingly difficult as the length of the polypeptide increases. Therefore, the presence of low-abundance deletions are common, expected, and accepted.

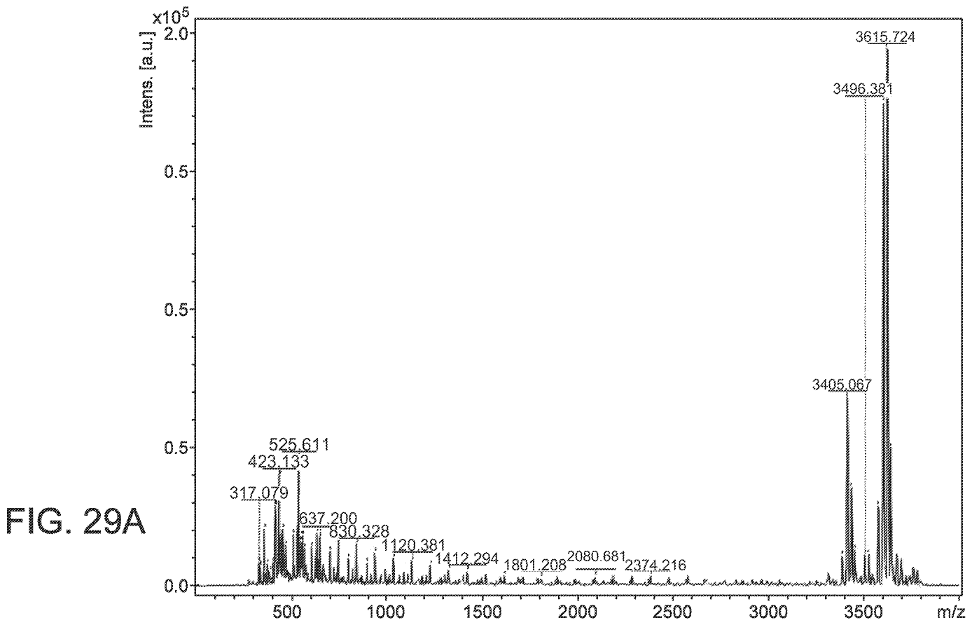

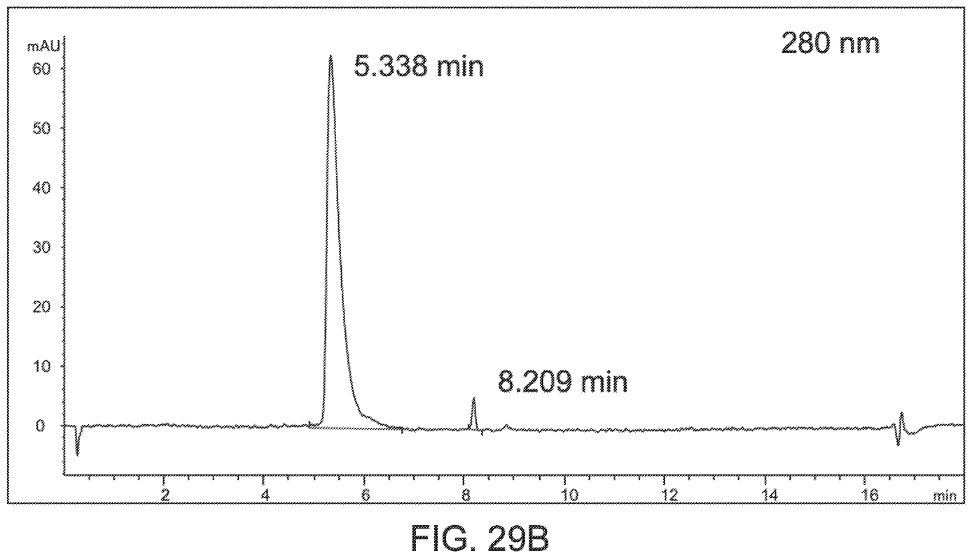

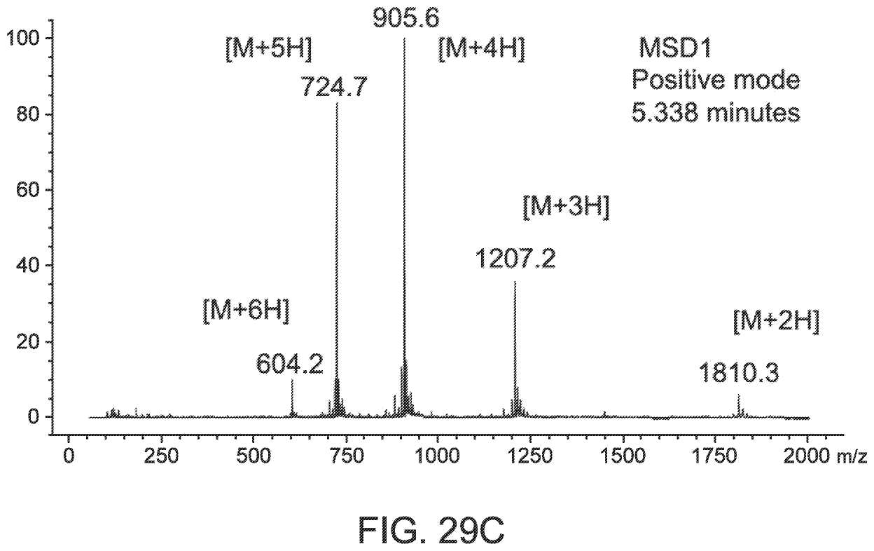

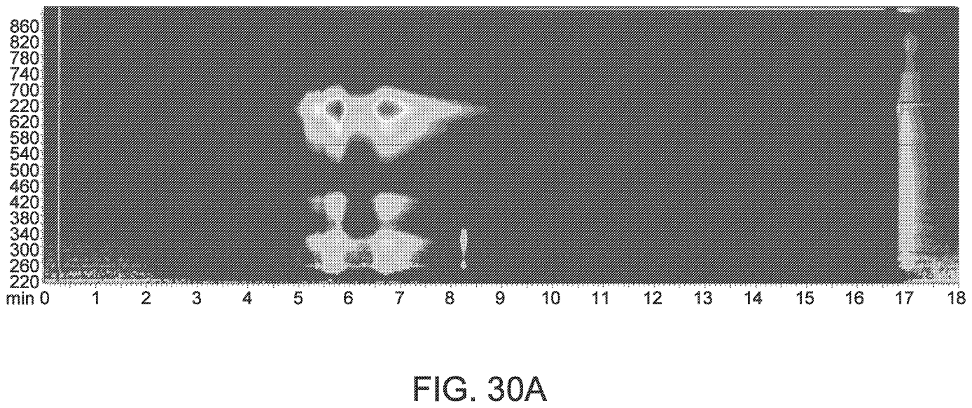

The presence of these deletions gives rise to a wide range of peaks around the target ion in ESI mass spectrometry, as shown in FIG. 29A-29C. There is very little evidence that these deletions noticeably impact downstream results.

### Example 13

### Conjugation of NHS Ester Dyes to Functionalized Polyproline (DBCO, Pro30)

### Aim:

To conjugate a commercially available fluorophore to a DBCO-functionalized polymer (or “Promer”). The final molecule being a functionalized fluorophore with an amino-acid based linker molecule and sequence from N terminus to C terminus being the following:

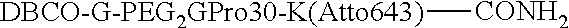

### Materials:

1. DBCO-G-PEG₂-G-Pro30-K (NH₂)—CONH₂//LNK-0D//Erisyon
2. HEPES buffer solution 1.0 M pH 8.5//J61360//Alfa Aesar
3. Acetonitrile//271004//Millipore Sigma
4. Formic acid//AC270480250//Fisher Scientific
5. Ammonium carbonate//036229-36//Thermo Scientific
6. Water

### Solutions Prepared:

1. 100 mM HEPES buffer
2. 0.2 M DBCO-G-PEG₂-G-Pro30-K—(NH₂)—CONH₂ in 50/50 ACN/100 mM HEPES (v/v)
3. 0.1 M Atto643-NHS in 50/50 ACN/100 mM HEPES (v/v)

### Instrument/Consumable Used:

1. Eppendorf Thermomixer C Model #: 5382
2. LC/MS instrumentation
   a. Agilent 1260 Infinity Degasser (G1322A)
   b. Agilent 1260 Infinity Binary Pump (G1312B)
   c. Agilent 1260 Infinity Sampler (G1329B) with external tray (p/n G1313-60004) and waste tube (p/n G1313-27302)
   d. Agilent 1260 Infinity Column Thermostat (G1316A) with a 2-position/6-port valve
   e. Agilent 1260 Infinity Diode Array Detector (G4212B)
   f. Agilent 6120B Single Quadrupole LC/MS (G6120B) with multimode source enabled with fast polarity switching for positive and negative mode acquisitions
   g. Agilent ZORBAX Eclipse Plus C18 narrow bore column; 2.1 mm internal diameter; 50 mm length; 5 micron particle size; P.N. 959746-902.

### Procedure:

1. Fix the commercially available fluorophore as the limiting reagent in the reaction.
2. Add 1.2 equivalents of the peptide solution to the commercially available fluorophore solution.
3. Allow the reaction to occur at 37° C. for 2 hours using an Eppendorf thermomixer.
4. Upon completion of the reaction, purify the sample via HPLC.
   a. Solvent A: 0.1% FA in H2O
   b. Solvent B: 0.1% FA in ACN
   c. Flow rate: 10 mL/min
   d. Column: Jupiter 5 μm C18, 300 A, 250×21.1 mm
   e. Gradient

| Time (min) | Solvent B % |
|---|---|
| 0.01 | 5 |
| 10.0 | 5 |
| 15.0 | 28 |
| 18.0 | 32 |
| 19.0 | 35 |
| 20.0 | 35 |
| 25.0 | 50 |
| 30.0 | 70 |
| 35.0 | 80 |
| 45.0 | 95 |
| 55.0 | 95 |

5. Immediately following elution from the HPLC, titrate the purified sample to neutral pH using ammonium carbonate.

### Supplementary Information

### DBCO Degradation and Mitigation

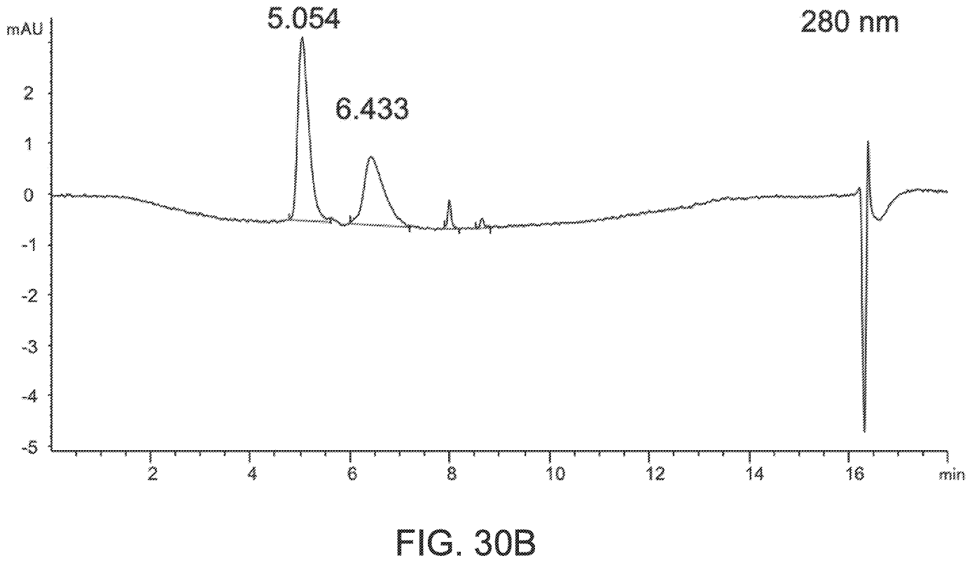

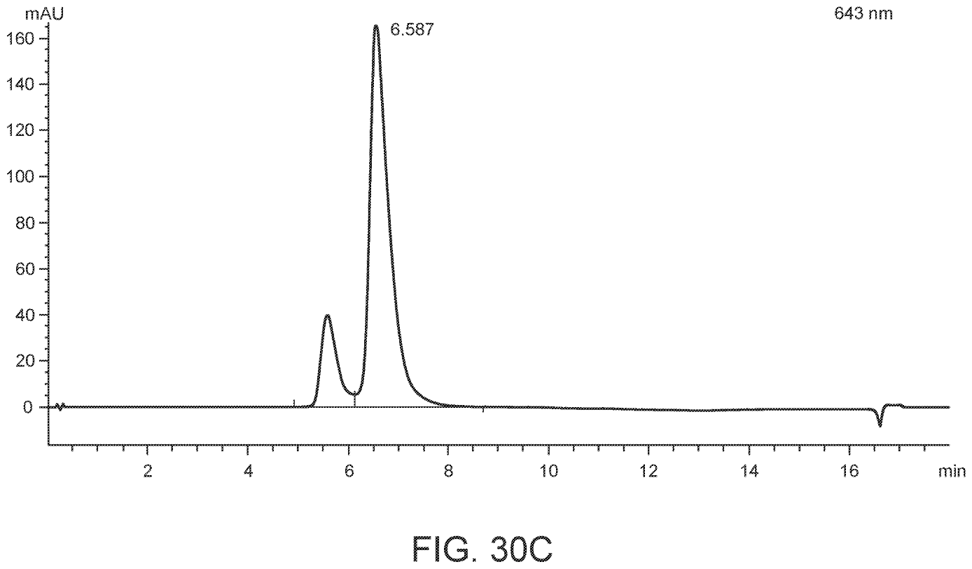

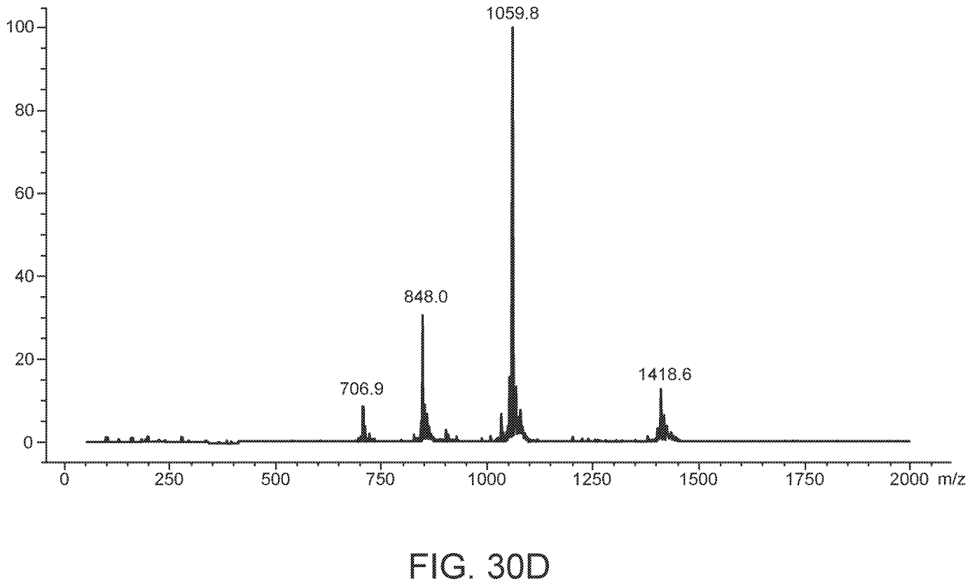

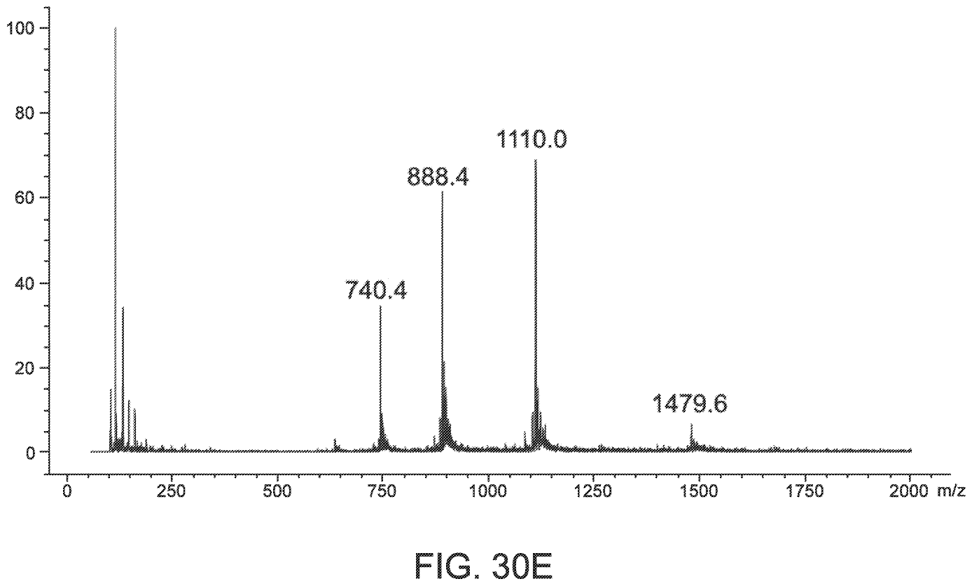

The DBCO functional group likely undergoes a 5-endo-dig cyclo-isomerization to form a small heterocycle in acidic conditions (seen at 8.209 minutes, FIG. 30B). This issue primarily interferes with HPLC purification.

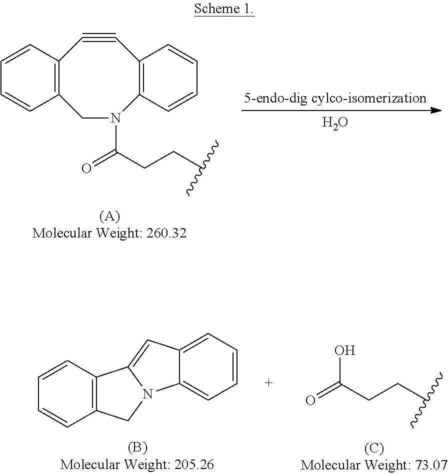

In order to mitigate this problem, neutralization via ammonium carbonate is employed following purification.
Neutralization via HEPES buffer has also been attempted. However, the addition of HEPES prior to lyophilization causes difficulties with solubility and salt concentrations. Ammonium bicarbonate is used due to its greater volatility.
Despite the purity of starting materials and the use of HEPES buffer in the reaction mixture, this side reaction still occurs in basic conditions upon reaction with Atto643-NHS ester. See FIG. 30.

### Mass Spectra Trends and Analysis.

The backbone of the polymer (or “promer”) technology is developed by utilizing solid phase peptide synthesis methods. Achieving sample homogeneity on solid-phase supports becomes increasingly difficult as the length of the polypeptide increases. Therefore, the presence of low-abundance deletions are common, expected, and accepted.
The presence of these deletions gives rise to a wide range of peaks around the target ion in ESI mass spectrometry. This phenomenon is demonstrated in the ESI mass spectra provided in, for example, FIGS. 30D and 30E. This trend can cause analysis via ESI alone to be very difficult and confusing. Utilizing MALDI and/or tandem LC/MS is recommended.

While specific embodiments have been illustrated and described, it will be readily appreciated that the various embodiments described above can be combined to provide further embodiments, and that various changes can be made therein without departing from the spirit and scope of the invention.

All of the U.S. patents, U.S. patent application publications, U.S. patent applications, foreign patents, foreign patent applications, and non-patent publications referred to in this specification, or listed in the Application Data Sheet, including U.S. Provisional Patent Application No. 63/412,780 filed Oct. 3, 2022, and U.S. Provisional Patent Application No. 63/582,766 filed Sep. 14, 2023, are incorporated herein by reference, in their entirety, unless otherwise stated. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A polymer, comprising:
a functional group configured to couple to a polypeptide or protein;
a backbone; and
an amino acid residue conjugated to a fluorophore.

2. The polymer of claim 1, wherein the backbone comprises a repeating monomer subunit.

3. The polymer of claim 1, wherein the backbone comprises:
(a) a rigid polypeptide comprising at least ten amino acid residues; or
(b) a rigid polypeptide comprising at least ten amino acid residues and a flexible spacer.

4. (canceled)

5. The polymer of claim 3, wherein;
(a) the polymer comprises the functional group, the flexible spacer, the rigid polypeptide and the amino acid residue conjugated to a fluorophore in amino-terminal to carboxy-terminal direction or carboxy-terminal to amino-terminal direction; and/or
(b) the polymer has a sequence of DBCO-Gly-(O—CH₂—CH₂)₂-Gly-Pro₃₀-Lys(fluorophore)-CONH₂ (SEQ ID NO:35).

6. The polymer of claim 3, wherein at least one amino acid residue of the at least ten amino acid residues of the rigid polypeptide is:
(a) a natural amino acid residue;
(b) an unnatural amino acid residue,
(c) an unnatural amino acid residue, wherein the unnatural amino acid residue is a proline residue derivative; and/or
(d) an unnatural amino acid residue, wherein the unnatural amino acid residue is a proline residue derivative that is hydroxyproline.

7.-9. (canceled)

10. The polymer of claim 1, wherein the polymer comprises or further comprises:
(a) an amino acid residue that is cationic, anionic, zwitterionic, or any combination thereof;
(b) an arginine, an alanine, glutamic acid, or any combination thereof;
(c) a phenylsulfonic acid, a polyglutamic acid, a polysarcosine, a polyalanine or any combination thereof;
(d) an antioxidant group;
(e) an antioxidant group comprising p-nitrophenylalanine, trolox, cyclooctatetraene, or any combination thereof, and/or
(f) a metal chelator.

11.-15. (canceled)

16. The polymer of claim 3, wherein the flexible spacer comprises:
(a) (O—CH₂—CH₂)ₙ;
(b) Gly-(O—CH₂—CH₂)ₙ-Gly;
(c) (O—CH₂—CH₂)ₙ, wherein n is a value between 1-23, inclusive;
(d) Gly-(O—CH₂—CH₂)ₙ-Gly, wherein n is a value between 1-23, inclusive;
(e) (O—CH₂—CH₂)ₙ, wherein n is 2;
(f) Gly-(O—CH₂—CH₂)ₙ-Gly, wherein nis 2;
(g) an alkyl chain;
(f) an alkyl chain, wherein the alkyl chain comprises 6-aminohexanoic acid, 12-aminododecanoic acid, or a combination thereof; and/or
(g) sarcosine (N-methylglycine) copolymerized with alanine, serine, or a combination thereof.

17.-22. (canceled)

23. The polymer of claim 3, wherein the rigid polypeptide comprises:
(a) at least ten proline residues, proline residue derivatives, or a combination thereof;
(b) from about ten to about 40 proline residues, proline residue derivatives, or a combination thereof;
(c) at least 14 proline residues, proline residue derivatives, or a combination thereof;
(d) at least 25 proline residues, proline residue derivatives, or a combination thereof;
(e) at least 30 proline residues, proline residue derivatives, or a combination thereof;
(f) a polyproline of between about ten and about 40 consecutive proline residues; and/or
(g) a polyproline of at least ten, at least 14, at least 25, at least 30, or 30 consecutive proline residues;
and/or the rigid polypeptide:
(i) comprises or consists of 30 proline residues, proline residue derivatives, or a combination thereof;
(ii) has a length from about 2 nm to about 12 nm; and/or
(iii) has a length from about 6 nm to about 9 nm.

24.-32. (canceled)

33. The polymer of claim 1, wherein the amino acid residue conjugated to the fluorophore:
(a) comprises a lysine residue, a cysteine residue, or an azidolysine residue; and/or
(b) is a lysine residue.

34. (canceled)

35. The polymer of claim 1, wherein the fluorophore:
(a) comprises an Alexa Fluor® dye, an Atto dye, a Janelia Fluor® dye, a carbopyronine derivative, a cyanine derivative, a rhodamine derivative, or any combination thereof;
(b) is Texas Red, Janelia Fluor® 525, Janelia Fluor® 549, Janelia Fluor® 555, Alexa Fluor® 448, Alexa Fluor® 555, Atto495, Atto643, Atto647N, AttoRho-12, Rhodamine, tetramethylrhodamine, or any combination thereof; and/or
(c) is Texas Red, Janelia Fluor® 549, Alexa Fluor® 555, Atto643, AttoRho-12, or any combination thereof.

36.-37. (canceled)

38. The polymer of claim 1, further comprising:
(a) at least one additional amino acid residue, wherein the additional amino acid residue is conjugated to an additional fluorophore;
(b) at least one additional amino acid residue, wherein the additional amino acid residue is conjugated to an additional fluorophore, wherein the additional amino acid residue conjugated to the additional fluorophore is positioned adjacent to the amino acid residue conjugated to the fluorophore;
(c) at least one additional amino acid residue, wherein the additional amino acid residue is conjugated to an additional fluorophore and the fluorophores are the same fluorophore; and/or
(d) at least one additional amino acid residue, wherein the additional amino acid residue is conjugated to an additional fluorophore and the fluorophores are different fluorophores.

39.-41. (canceled)

42. The polymer of claim 1, wherein the functional group:
(a) is a strained alkyne, a iodoacetamide, a maleimide, an amine, an azide, an alkene, an aldehyde, a ketone, a tetrazine, an alkyne, a cycloalkyne, a cyclooctyne, dibenzocyclooctyne (DBCO), a thiol, a carboxyl, a hydrazide, a dithiol, a trans-cyclooctene, a bicycloalkene, an iodobenzene, a cyanothiazole, an acene, a dithiolane, a bromane, an aminothiol, a pyrroledione, a sulfenyl chloride, a succinimidyl ester, a succinidimyl ester, methyltetrazine, lipoic acid, or any combination thereof,
(b) is a strained alkyne;
(c) is dibenzocyclooctyne (DBCO), methyltetrazine, or lipoic acid,
(d) is dibenzocyclooctyne (DBCO);
(e) comprises a clack group; and/or
(f) comprises a clack group, wherein the clack group is configured to react with a click group on a peptide.

43.-47. (canceled)

48. A polymer, comprising in amino-terminal to carboxy-terminal direction:
a functional group that is dibenzocyclooctyne (DBCO);
a flexible spacer comprising Gly-(O—CH₂—CH₂)₂-Gly;
a rigid polypeptide comprising 30 proline residues; and
a lysine residue conjugated to a fluorophore.

49. A polymer comprising or consisting of structure I: 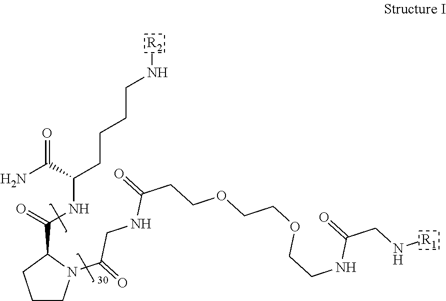
wherein R₁=DBCO and R₂=a fluorophore.

50.-51. (canceled)

52. A peptide-polymer conjugate comprising:
the polymer of claim 1; and
a peptide with at least one amino-acid side chain, wherein the amino-acid side chain is attached to the polymer via the functional group.

53. The peptide-polymer conjugate of claim 52, wherein:
(a) at least two polymers are attached to the peptide via two different amino-acid side chains;
(b) at least two polymers are attached to the peptide via two different amino-acid side chains, wherein the at least two polymers comprise the same fluorophore, and dye quenching between the fluorophores is reduced compared to dye quenching between identical fluorophores attached directly to the amino-acid side chains of the peptide; and/or
(c) at least two polymers are attached to the peptide via two different amino-acid side chains, wherein the at least two polymers comprise different fluorophores, and wherein Förster resonance energy transfer (FRET) between the fluorophores is reduced compared to FRET between identical fluorophores attached directly to the amino-acid side chains of the peptide.

54.-55. (canceled)

56. A composition comprising at least one polymer of claim 1 and a solvent.

57. A method of reducing dye-dye interactions, comprising:
a) providing the polymer of claim 1;
b) providing a biomolecule comprising at least two reactive groups; and
c) attaching at least two polymers to the at least two reactive groups of the biomolecule via the functional group, wherein the two polymers comprise the same fluorophore;
thereby reducing dye-dye interactions between identical fluorophores compared to dye-dye interactions between identical fluorophores conjugated directly to the at least two reactive groups of the biomolecule.

58. The method of claim 57, wherein:
(a) the biomolecule is a peptide;
(b) the reactive group is an amino-acid side chain; and/or
(c) dye-dye interactions are reduced by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to dye-dye interactions between identical fluorophores conjugated directly to the at least two reactive groups of the biomolecule.

59.-60. (canceled)

61. A method of reducing dye quenching, comprising:
a) providing the polymer of claim 1;
b) providing a peptide with at least two amino-acid side chains; and
c) attaching at least two polymers to the at least two amino-acid side chains via the functional group, wherein the two polymers comprise the same fluorophore;
thereby reducing dye quenching between identical fluorophores compared to dye quenching between identical fluorophores conjugated directly to the at least two amino-acid side chains of the peptide.

62. The method of claim 61, wherein;
(a) the identical fluorophores are Atto647N;
(b) dye quenching is reduced by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to dye quenching between identical fluorophores conjugated directly to the at least two amino-acid side chains of the peptide; and/or
(c) dye quenching is reduced by at least about 50%.

63.-64. (canceled)

65. A method of reducing Förster resonance energy transfer (FRET), comprising:
a) providing the polymer of claim 1;
b) providing a peptide with at least two amino-acid side chains; and
c) attaching at least two polymers to the at least two amino-acid side chains via the functional group, wherein the two polymers comprise different fluorophores;
thereby reducing FRET between the fluorophores compared to FRET between equivalent fluorophores conjugated directly to the at least two amino-acid side chains of the peptide.

66. The method of claim 65, wherein;
(a) the different fluorophores are Atto647N and Janelia Fluor® 549,
(b) the different fluorophores are Atto643 and AttoRho-12;
(c) FRET is reduced by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90% or about 95% compared to FRET between equivalent fluorophores conjugated directly to the at least two amino-acid side chains of the peptide; and/or
(d) FRET is reduced by at least about 50%.

67.-68. (canceled)

69. A method for labeling an amino acid of a peptide, the method comprising:
(a) providing the peptide, wherein the peptide comprises an internal amino acid coupled to an azide and a C-terminus coupled to an alkyne; and
(b) bringing the peptide in contact with a first polymer of claim 1 under conditions such that the first polymer reacts with the internal amino acid, wherein the first polymer comprises a functional group that is a strained alkyne.

70. The method of claim 69, wherein (b) is performed in the absence of copper (Cu).

71. The method of claim 69, further comprising, (c) reacting a second polymer different from the first polymer with said C-terminus, wherein the second polymer comprises:
the functional group that is a non-strained alkyne configured to couple to the polypeptide or protein;
the backbone; and
the amino acid residue conjugated to the fluorophore.

72. The method of claim 71, wherein (c) is performed in the presence of copper (Cu).

73. The method of claim 69, wherein the azide coupled to the internal amino acid does not react with the alkyne coupled to the C-terminus.


SEQUENCE LISTING (48 sequences)

SEQ ID NO: 1 - protein, 14 aa, synthetic construct
KDDYAGGGAAGKDA

SEQ ID NO: 2 - protein, 18 aa, synthetic construct
  SITE 16: note = Propargylglycine
AGKGAAGGAANSYRNXRN

SEQ ID NO: 3 - protein, 18 aa, synthetic construct
  SITE 16: note = Propargylglycine
APKGAAGGAANSYRNXRN

SEQ ID NO: 4 - protein, 18 aa, synthetic construct
  SITE 16: note = Propargylglycine
GAKGAAGGAANSYRNXRN

SEQ ID NO: 5 - protein, 18 aa, synthetic construct
  SITE 16: note = Propargylglycine
AVKGAAGGAANSYRNXRN

SEQ ID NO: 6 - protein, 18 aa, synthetic construct
  SITE 16: note = Propargylglycine
AAKGAAGGAANSYRNXRN

SEQ ID NO: 7 - protein, 17 aa, synthetic construct
  SITE 2: note = azido-lysine
  SITE 6: note = azido-lysine
  SITE 8: note = azido-lysine
  SITE 15: note = Propargylglycine
GXGAAXGXANSYRNXRN

SEQ ID NO: 8 - protein, 9 aa, synthetic construct
  SITE 3: note = azido-lysine
  SITE 8: note = Propargylglycine
GKXAGAYXR

SEQ ID NO: 9 - protein, 11 aa, synthetic construct
  SITE 2: note = azido-lysine
  SITE 5: note = azido-lysine
  SITE 10: note = Propargylglycine
GXAGXAGAYXR

SEQ ID NO: 10 - protein, 19 aa, synthetic construct
  SITE 6: note = azido-lysine
  SITE 18: note = Propargylglycine
GKGASXGANSAKGSYRNXR

SEQ ID NO: 11 - protein, 9 aa, synthetic construct
  SITE 3: note = azido-lysine
  SITE 8: note = Propargylglycine
GKXAGAYXR

SEQ ID NO: 12 - protein, 11 aa, synthetic construct
  SITE 5: note = azido-lysine
  SITE 10: note = Propargylglycine
GKAGXAGSYXR

SEQ ID NO: 13 - protein, 11 aa, synthetic construct
  SITE 2: note = azido-lysine
  SITE 5: note = azido-lysine
  SITE 10: note = Propargylglycine
GXAGXAGAYXR

SEQ ID NO: 14 - protein, 12 aa, synthetic construct
  SITE 6: note = azido-lysine
  SITE 11: note = Propargylglycine
ELYAEXVATRXG

SEQ ID NO: 15 - protein, 17 aa, synthetic construct
  SITE 15: note = Propargylglycine
AKGAAGGAANSYRNXRN

SEQ ID NO: 16 - protein, 17 aa, synthetic construct
  SITE 15: note = Propargylglycine
AKGAAGGAANSYRNXRN

SEQ ID NO: 17 - protein, 17 aa, synthetic construct
  SITE 15: note = Propargylglycine
AKGAAGGAANSYRNXRN

SEQ ID NO: 18 - protein, 18 aa, synthetic construct
  SITE 16: note = Propargylglycine
AGKGAAGGAANSYRNXRN

SEQ ID NO: 19 - protein, 18 aa, synthetic construct
  SITE 16: note = Propargylglycine
APKGAAGGAANSYRNXRN

SEQ ID NO: 20 - protein, 18 aa, synthetic construct
  SITE 16: note = Propargylglycine
GAKGAAGGAANSYRNXRN

SEQ ID NO: 21 - protein, 18 aa, synthetic construct
  SITE 16: note = Propargylglycine
AVKGAAGGAANSYRNXRN

SEQ ID NO: 22 - protein, 18 aa, synthetic construct
  SITE 16: note = Propargylglycine
AAKGAAGGAANSYRNXRN

SEQ ID NO: 23 - protein, 19 aa, synthetic construct
  SITE 17: note = Propargylglycine
APAKGAAGGAANSYRNXRN

SEQ ID NO: 24 - protein, 19 aa, synthetic construct
  SITE 17: note = Propargylglycine
AAPKGAAGGAANSYRNXRN

SEQ ID NO: 25 - protein, 18 aa, synthetic construct
  SITE 16: note = Propargylglycine
PAKGAAGGAANSYRNXRN

SEQ ID NO: 26 - protein, 10 aa, synthetic construct
  SITE 9: note = Propargylglycine
GKGSKSYNXR

SEQ ID NO: 27 - protein, 17 aa, synthetic construct
  SITE 15: note = Propargylglycine
AKGAAKGKANSYRNXRN

SEQ ID NO: 28 - protein, 17 aa, synthetic construct
  SITE 15: note = Propargylglycine
AKGKAKGKANSYRNXRN

SEQ ID NO: 29 - protein, 11 aa, synthetic construct
GKAGCAGAYGR

SEQ ID NO: 30 - protein, 9 aa, synthetic construct
  SITE 8: note = Propargylglycine
GKAGCAGXR

SEQ ID NO: 31 - protein, 11 aa, synthetic construct
  SITE 10: note = Propargylglycine
GKAGCAGAYXR

SEQ ID NO: 32 - protein, 6 aa, synthetic construct
  SITE 5: note = Propargylglycine
  SITE 3: note = azido-lysine
KGXGXG

SEQ ID NO: 33 - protein, 9 aa, synthetic construct
  SITE 3: note = azido-lysine
  SITE 8: note = Propargylglycine
GKXAGAYXR

SEQ ID NO: 34 - protein, 18 aa, synthetic construct
  SITE 16: note = Propargylglycine
APKGAAGGAANSYRNXRN

SEQ ID NO: 35 - protein, 33 aa, synthetic construct
GGPPPPPPPPPPPPPPPPPPPPPPPPPPPPPPK

SEQ ID NO: 36 - protein, 8 aa, synthetic construct
GCKAGAYR

SEQ ID NO: 37 - protein, 9 aa, synthetic construct
GKAGKAGAY

SEQ ID NO: 38 - protein, 9 aa, synthetic construct
GKAGCAGSY

SEQ ID NO: 39 - protein, 17 aa, synthetic construct
GKGASCGANSAKGSYRN

SEQ ID NO: 40 - protein, 20 aa, synthetic construct
KSPVSLAQYWWNQINPFYQM

SEQ ID NO: 41 - protein, 20 aa, synthetic construct
RGAWMKRYKGAWPQWDANCS

SEQ ID NO: 42 - protein, 20 aa, synthetic construct
FSQQETECKYMPFPARNLDM

SEQ ID NO: 43 - protein, 20 aa, synthetic construct
SCKPARPKGFLQHPWDYAPD

SEQ ID NO: 44 - protein, 20 aa, synthetic construct
MDKYHNVQWPCIFAPAGRKC

SEQ ID NO: 45 - protein, 20 aa, synthetic construct
CQVRHCFGSMQDMWHPIAHM

SEQ ID NO: 46 - protein, 20 aa, synthetic construct
PQMLTDSATKKPFDVVHIPF

SEQ ID NO: 47 - protein, 20 aa, synthetic construct
TPCFGEKYANVGDHTWWMHW

SEQ ID NO: 48 - protein, 35 aa, synthetic construct
GGRRPPPPPPPPPPPPPPPPPPPPPPPPPPPPPPK